(19) United States
(12) Patent Application Publication
(10) Pub. No.: US 2026/0356834 A1
(43) Pub. Date: Nov. 12, 2026

(54) ANTICONVULSIVE COMPOUNDS AND USES THEREOF

(71) Applicant: Jazz Pharmaceuticals Research UK Limite, Sittingbourne, Kent (GB)

(72) Inventors: Paolo DI FRUSCIA, Sittingbourne, Kent (GB); Paul Stuart HINCHLIFFE, Cambridge (GB); lain David SIMPSON, Cambridge (GB); Joanne PEACH, Cambridge (GB)

(21) Appl. No.: 19/483,577

(22) PCT Filed: May 10, 2024
(86) PCT No.: PCT/GB2024/051216

§ 371 Date: Nov. 11, 2025

(30) Foreign Application Priority Data
May 12, 2023 (GB) 2307065.9

(51) Int. Cl. C07D 471/04; A61K 31/437; A61P 25/08

(52) U.S. Cl. CPC C07D 471/04; A61K 31/437; A61P 25/08

(57) ABSTRACT

The present invention relates to a group of synthetic cannabinoid compounds, which have the structural formula I defined herein. The present invention also relates to compositions comprising these compounds, methods of preparing these compounds, intermediates useful in the preparation of these compounds, and to the use of these compounds as medicaments, especially for the treatment of conditions associated with seizure, such as epilepsy.

### RELATED APPLICATIONS

The present application is related to, and claims the benefit of, GB2307065.9 filed on 12 May 2023 (Dec. 5, 2023), the contents of which are hereby incorporated by reference in their entirety.

### FIELD OF THE INVENTION

The present invention relates to a group of synthetic cannabinoid compounds and to the use of these compounds as research tools and medicaments, especially for the treatment of conditions associated with seizure, such as epilepsy.

### BACKGROUND TO THE INVENTION

Epilepsy is a disabling neurological disorder, characterized by a predisposition to epileptic seizures caused by abnormal, excessive, or synchronous neuronal activity in the brain. Epilepsy sufferers may experience a substantially impaired quality of life as a result of seizures, comorbid mood and psychiatric disorders, cognitive deficits and the adverse effects of epilepsy medication [Devinsky et al, 2018]. Furthermore, seizures can be fatal, either directly due to effects on autonomic and arousal functions, or indirectly due to accidents such as drowning, or motor vehicle accidents.

Epilepsy is reported to affect 65 million people worldwide [Devinsky et al, 2018], with a lifetime prevalence of 7.6 per 1,000 people [Fiest et al, 2017]. Whilst the prevalence does not appear to differ by age group or gender, the prevalence and incidence rate of epilepsy are higher in low to middle income countries [Fiest et al, 2017].

The causes of epilepsy include acquired structural changes to the brain (e.g. as a result of traumatic brain injury, brain tumour, or stroke), infectious diseases (e.g. viral or bacterial disorders), and genetic mutations.

The International League Against Epilepsy (ILAE) epilepsy classification framework begins with the diagnosis of the type of epileptic seizure and assumes that non-epileptic events have been ruled out. The classification proposes a three-level classification approach for a patient presenting with epileptic seizures, wherein the first level is seizure type, the second level is epilepsy type and the third level is an epilepsy syndrome diagnosis [Scheffer et al, 2017].

Seizure type classification begins with the determination of whether the initial manifestations of the seizure are of focal onset or generalised onset. If the onset is missed or obscured the seizure is categorised as being of unknown onset [Fisher et al, 2017]. Focal onset seizures may be further defined according to the level of awareness and all three seizure types (focal, generalised and unknown) may be further characterised by motor or nonmotor onset symptoms.

Epilepsy type classification divides into focal epilepsies, generalised epilepsies, combined generalised & focal epilepsies, and unknown epilepsies. Focal epilepsies include unifocal and multifocal disorders as well as seizures involving one hemisphere. For a diagnosis of Generalised Epilepsy, the patient would typically show generalised spike-wave activity on EEG. Individuals with generalised epilepsies may have a range of seizure types including absence, myoclonic, atonic, tonic, and tonic-clonic seizures. Combined Generalised and Focal Epilepsies covers patients who have both generalised and focal seizures, whereas unknown epilepsies covers situations where the patient has epilepsy but the clinician is unable to determine if the epilepsy type is focal or generalised, because there is insufficient information available [Scheffer et al., 2017].

Epilepsy syndrome refers to a characteristic cluster of clinical and EEG features, often supported by specific etiological findings (structural, genetic, metabolic, immune, and infectious) [Wirrell et al., 2022]. The epilepsy syndrome groups include syndromes with onset in neonates and infancy (up to 24 months of age); syndromes with onset in childhood; syndromes with onset at a variable age; and idiopathic generalised epilepsy syndromes (IGEs). The syndrome groups may also encompass self-limited epilepsies, developmental and epileptic encephalopathies (DEE) and aetiology-specific syndromes.

Examples of neonate and infant epilepsy syndromes include self-limited neonatal epilepsy, infantile epileptic spasms syndrome, Dravet syndrome, glucose transporter 1 deficiency syndrome and Sturge Weber syndrome [Zuberi et al., 2022].

Examples of syndromes with onset in childhood include childhood occipital visual epilepsy (COVE), Lennox-Gastaut syndrome (LGS), epilepsy with myoclonic atonic seizures (previously known as Doose syndrome) and epilepsy with eyelid myoclonia (previously known as Jeavons syndrome) [Specchio et al., 2022].

Examples of syndromes with onset at a variable age include sleep-related hypermotor/hyperkinetic epilepsy (SHE), progressive myoclonus epilepsies (PME), febrile-infection related epilepsy syndrome (FIRES) and Rasmussen syndrome [Riney et al., 2022].

Idiopathic generalised epilepsy syndromes (IGEs) include childhood absence epilepsy (CAE), juvenile absence epilepsy (JAE), juvenile myoclonic epilepsy (JME), and epilepsy with generalised tonic-clonic seizures alone (GTCA) [Hirsch et al., 2022].

In terms of the management of epilepsy, options include anti-seizure drugs (ASDs), dietary therapies, neurostimulation and surgery. ASDs are the primary therapy for epilepsy and aim to reduce the seizure occurrence and severity. Over 20 ASDs have been approved, however, often multiple drugs are required for adequate seizure control. Strict patient adherence to their ASD treatment regimen is also important, as ASDs must be taken between one and four times daily and a single missed dose can lead to recurrence of seizures [Devinsky et al, 2018].

Cannabidiol (CBD) is one such ASD. In 2018 CBD (Epidiolex®) was approved in the United States for the treatment of Dravet syndrome and Lennox-Gastaut syndrome in children and adults over the age of 2 years. CBD is a naturally occurring cannabinoid compound derived from cannabis species, such as the hemp plant (Cannabis sativa). Unlike other cannabinoids, such as tetrahydrocannabinol (THC), CBD does not bind to CB1 or CB2 receptors (or its binding to the receptors is negligible in terms of inducing a pharmacological effect) and it does not induce the psychotropic effects associated with cannabis.

Despite the wide range of ASDs available, approximately one-third of patients do not achieve seizure control, no matter how many ASDs are tried using monotherapy or combination drugs [Devinsky et al, 2018]. Drug- or treatment-resistant epilepsy is associated with increased risk of injury and death, greater medication burden and adverse effects, and reduced quality-of-life. For these patients, surgery offers the greatest chance of achieving long-term seizure control, but only a minority of patients are good candidates for surgery. Clearly, alternative treatment options for seizures are desirable.

The present invention has been devised in light of the above considerations.

### BRIEF SUMMARY OF THE INVENTION

At its most general, the present invention relates to synthetic cannabinoid compounds. The synthetic cannabinoid compounds display anticonvulsive activity in one or more rodent models of seizure and hence the compounds will be useful for the treatment or prevention of medical conditions such as epilepsy.

In a first aspect of the invention there is provided a compound of formula (I), or a salt thereof:

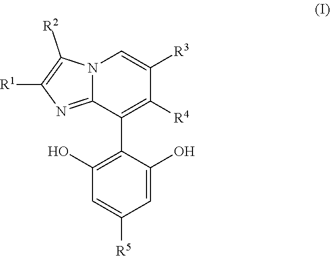

wherein,
R¹ is hydrogen, C₁₋₃alkyl, C₁₋₃alkoxy, C₁₋₃haloalkyl, or halo;
R² is hydrogen, C₁₋₃alkyl, C₁₋₃alkoxy, or halo;
R³ is hydrogen, C₁₋₃alkyl, C₁₋₃alkoxy, C₁₋₃haloalkyl, C₃₋₆cycloalkyl, or halo;
R⁴ is hydrogen, C₁₋₃alkyl, C₁₋₃alkoxy, C₁₋₃haloalkyl, C₃₋₆cycloalkyl, or halo; and
R⁵ is hydrogen, C₁₋₁₀alkyl, C₁₋₁₀haloalkyl, C₃₋₆cycloalkyl, or halo.

In a second aspect of the invention, there is provided a pharmaceutical composition comprising a compound of the first aspect, or a pharmaceutically acceptable salt thereof, together with one or more ingredients selected from carriers, diluents, excipients, adjuvants, fillers, buffers, binders, disintegrants, preservatives, antioxidants, lubricants, stabilisers, solubilisers, surfactants (e.g., wetting agents), masking agents, colouring agents, flavouring agents, and sweetening agents.

In a third aspect of the invention, there is provided a compound of the first aspect, or the pharmaceutical composition of the second aspect, for use in a method of treatment.

In an embodiment of the third aspect, there is provided a compound of the first aspect, or the pharmaceutical composition of the second aspect, for use in the treatment of a condition associated with seizure.

In a fourth aspect of the invention, there is provided a method of treatment comprising administering to a subject in need of treatment a therapeutically effective amount of a compound of the first aspect, or the pharmaceutical composition of the second aspect.

In an embodiment of the fourth aspect, the treatment comprises the treatment of a condition associated with seizure.

In a fifth aspect of the invention, there is provided a method of preparing a compound of the first aspect, the method comprising:
(1a) reacting a compound of formula (II) with a compound of formula (III) to give a compound of formula (IV):

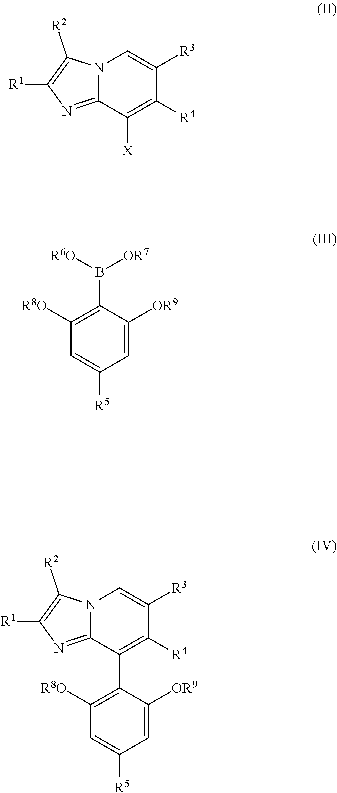

where:
R¹, R², R³, R⁴, and R⁵ are as defined herein;
X is chloro, bromo, iodo, or triflate;
R⁶ and R⁷ are hydrogen, alkyl or phenyl; or R⁶ and R⁷ are joined to form a cyclic boronic ester (such as pinacol, neopentyl or catechol boronic esters); and
R⁸ and R⁹ are suitable protecting groups such as methyl or benzyl; and
(1b) converting a compound of formula (IV) to a compound of formula (I).

In a sixth aspect of the invention, there are provided intermediates useful in the preparation of a compound of the first aspect, wherein the intermediates are compounds of formula (II) or formula (IV):

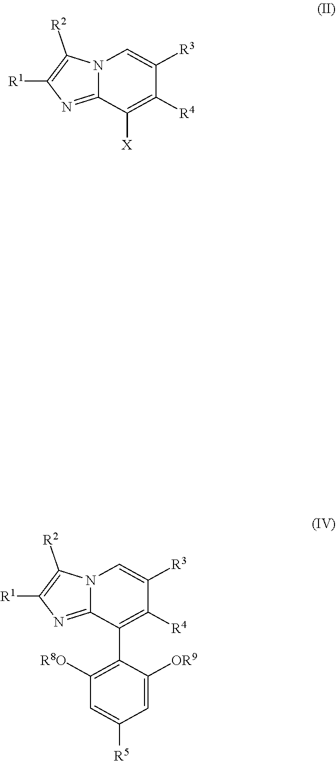

wherein R¹ is hydrogen, C₁₋₃alkyl, C₁₋₃alkoxy, C₁₋₃haloalkyl, or halo;
R² is hydrogen, C₁₋₃alkyl, C₁₋₃alkoxy, or halo;
R³ is hydrogen, C₁₋₃alkyl, C₁₋₃alkoxy, C₁₋₃haloalkyl, C₃₋₆cycloalkyl, or halo;
R⁴ is hydrogen, C₁₋₃alkyl, C₁₋₃alkoxy, C₁₋₃haloalkyl, C₃₋₆cycloalkyl, or halo; X is chloro, bromo, iodo, or triflate;
R⁵ is hydrogen, C₁₋₁₀alkyl, C₁₋₁₀haloalkyl, C₃₋₆cycloalkyl, or halo; and
R⁸ and R⁹ are suitable protecting groups such as methyl or benzyl.

These and other aspects and embodiments of the invention are described in further detail below.

### DETAILED DESCRIPTION OF THE INVENTION

The present invention relates to synthetic cannabinoid compounds which are biologically active and hence useful in the treatment of diseases.

The compounds are structurally related to the naturally-occurring cannabinoid cannabidiol (CBD). CBD is a non-psychoactive cannabinoid which has been used to treat various diseases and disorders. While such treatments hold promise, there remains a need in the art for more effective treatments and this has been brought about by way of novel synthetic cannabinoid compounds.

### Synthetic Cannabinoids

Natural cannabidiol (CBD) has the formula:

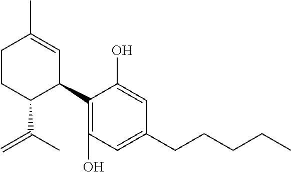 [SMILES: C=C(C)[C@@H]1CCC(C)=C[C@H]1c1c(O)cc(CCCCC)cc1O]

### Compounds of Formula (I)

In one aspect, the present invention provides a compound of formula (I), or a salt thereof:

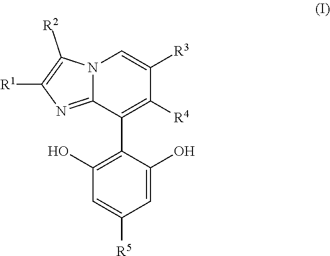

wherein,
R¹ is hydrogen, C₁₋₃alkyl, C₁₋₃alkoxy, C₁₋₃haloalkyl, or halo;
R² is hydrogen, C₁₋₃alkyl, C₁₋₃alkoxy, or halo;
R³ is hydrogen, C₁₋₃alkyl, C₁₋₃alkoxy, C₁₋₃haloalkyl, C₃₋₆cycloalkyl, or halo;
R⁴ is hydrogen, C₁₋₃alkyl, C₁₋₃alkoxy, C₁₋₃haloalkyl, C₃₋₆cycloalkyl, or halo; and
R⁵ is hydrogen, C₁₋₁₀alkyl, C₁₋₁₀haloalkyl, C₃₋₆cycloalkyl, or halo.

In an embodiment, R¹ is hydrogen, C₁₋₃alkyl, C₁₋₃haloalkyl, or halo. In an embodiment, R¹ is hydrogen, methyl, CF₃, fluoro, or chloro. In an embodiment, R¹ is hydrogen, methyl, CF₃, or chloro. In an embodiment, R¹ is hydrogen or methyl. In a more convenient embodiment, R¹ is hydrogen.

In an embodiment, R² is hydrogen, C₁₋₃alkyl, or halo. In an embodiment, R² is hydrogen, methyl, fluoro or chloro. In an embodiment, R² is hydrogen, methyl, or chloro. In an embodiment, R² is hydrogen or methyl. In a more convenient embodiment, R² is hydrogen.

In an embodiment, R³ is hydrogen, C₁₋₃alkyl, C₁₋₃haloalkyl, C₃₋₆cycloalkyl, or halo. In an embodiment, R³ is hydrogen, methyl, CF₃, cyclopropyl, fluoro, or chloro. In a more convenient embodiment, R³ is hydrogen, fluoro, or chloro. In a yet more convenient embodiment, R³ is chloro.

In an embodiment, R⁴ is hydrogen, C₁₋₃alkyl, C₁₋₃haloalkyl, C₃₋₆cycloalkyl, or halo. In an embodiment, R⁴ is hydrogen, methyl, CF₃, cyclopropyl, fluoro, or chloro. In a more convenient embodiment, R⁴ is methyl, CF₃, cyclopropyl, fluoro, or chloro. In a yet more convenient embodiment, R⁴ is methyl.

In an embodiment, R⁵ is C₁₋₁₀alkyl, C₁₋₁₀haloalkyl, C₃₋₆cycloalkyl, or halo. In an embodiment, R⁵ is hydrogen, C₃₋₆alkyl, C₃₋₆haloalkyl, C₃₋₆cycloalkyl, or halo. In an embodiment, R⁵ is C₃₋₆alkyl, C₃₋₆haloalkyl, C₃₋₆cycloalkyl, or halo. In an embodiment, R⁵ is C₃₋₆alkyl, C₃₋₆haloalkyl, C₃₋₆cycloalkyl, or chloro. In an embodiment, R⁵ is C₁₋₁₀alkyl, C₁₋₁₀haloalkyl, or C₃₋₆cycloalkyl. In an embodiment, R⁵ is C₃₋₆alkyl, C₃₋₆haloalkyl, or C₃₋₆cycloalkyl. In an embodiment, R⁵ is hydrogen, C₃₋₅alkyl, C₃₋₅haloalkyl, cyclopentyl, or chloro. In an embodiment, R⁵ is C₃₋₅alkyl, C₃₋₅haloalkyl, cyclopentyl, or chloro. In a more convenient embodiment, R⁵ is hydrogen, propyl, pentyl, 1,1-dimethylpropyl (tert-pentyl), 3,3,3-trifluoropropyl, cyclopentyl, or chloro. In a more convenient embodiment, R⁵ is propyl, pentyl, 1,1-dimethylpropyl (tert-pentyl), 3,3,3-trifluoropropyl, cyclopentyl, or chloro. In a yet more convenient embodiment, R⁵ is C₁₋₁₀alkyl, such as C₃₋₆alkyl, or C₃₋₅alkyl. In a most convenient embodiment, R⁵ is propyl or pentyl. In a most convenient embodiment, R⁵ is propyl. In a most convenient embodiment, R⁵ is pentyl.

In a convenient embodiment, the compound of formula I has one of the structural formulae IA to IF (sub-structures of formula I), or a salt thereof:

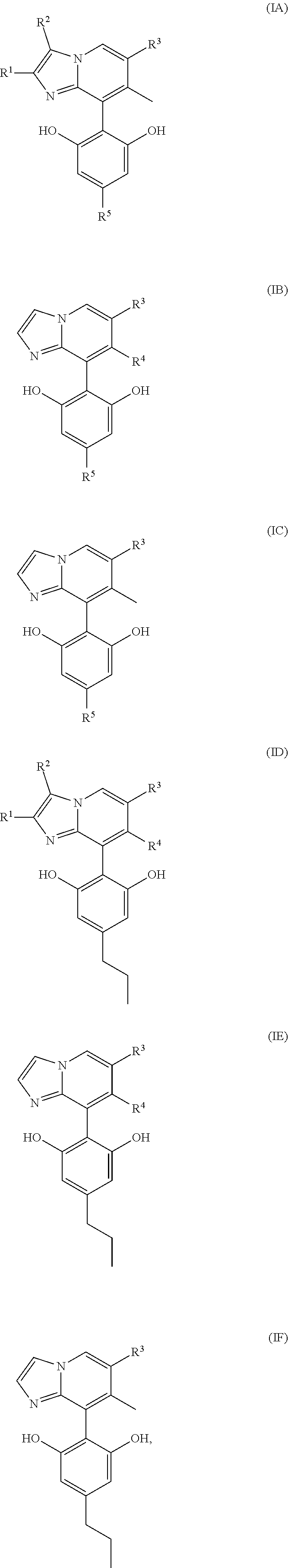

wherein R¹, R², R³, R⁴, and R⁵ are as defined in any of the embodiments hereinabove.

In an embodiment, there is provided a compound of formula (IC), or a salt thereof, wherein R³ is hydrogen, methyl, CF₃, cyclopropyl, fluoro, or chloro; and R⁵ is hydrogen, C₃₋₆alkyl, C₃₋₆haloalkyl, C₃₋₆cycloalkyl, or halo. In an embodiment, there is provided a compound of formula (IC), or a salt thereof, wherein R³ is hydrogen, fluoro, or chloro; and R⁵ is C₃₋₆alkyl, or C₃₋₆haloalkyl. In an embodiment, there is provided a compound of formula (IC), or a salt thereof, wherein R³ is hydrogen, fluoro, or chloro; and R⁵ is C₃₋₆alkyl (such as propyl).

In an embodiment, there is provided a compound of formula (IF), or a salt thereof, wherein R³ is hydrogen, methyl, CF₃, cyclopropyl, fluoro, or chloro. In an embodiment, there is provided a compound of formula (IF), or a salt thereof, wherein R³ is hydrogen, fluoro, or chloro.

In an embodiment, the compound of formula (I) is selected from the following compounds and salts thereof:
2-(imidazo[1,2-a]pyridin-8-yl)-5-propylbenzene-1,3-diol;
2-(7-methylimidazo[1,2-a]pyridin-8-yl)-5-propylbenzene-1,3-diol;
2-(2,7-dimethylimidazo[1,2-a]pyridin-8-yl)-5-propylbenzene-1,3-diol;
2-(6-chloro-7-methylimidazo[1,2-a]pyridin-8-yl)-5-propylbenzene-1,3-diol;
2-(7-trifluoromethylimidazo[1,2-a]pyridin-8-yl)-5-propylbenzene-1,3-diol;
2-(7-chloroimidazo[1,2-a]pyridin-8-yl)-5-propylbenzene-1,3-diol;
2-(6,7-dimethylimidazo[1,2-a]pyridin-8-yl)-5-propylbenzene-1,3-diol;
2-(7-fluoroimidazo[1,2-a]pyridin-8-yl)-5-propylbenzene-1,3-diol;
2-(3,7-dimethylimidazo[1,2-a]pyridin-8-yl)-5-propylbenzene-1,3-diol;
2-(7-cyclopropylimidazo[1,2-a]pyridin-8-yl)-5-propylbenzene-1,3-diol;
2-(3-chloro-7-methylimidazo[1,2-a]pyridin-8-yl)-5-propylbenzene-1,3-diol;
2-(6-fluoro-7-methylimidazo[1,2-a]pyridin-8-yl)-5-propylbenzene-1,3-diol;
2-(2-chloro-7-methylimidazo[1,2-a]pyridin-8-yl)-5-propylbenzene-1,3-diol;
2-(7-methylimidazo[1,2-a]pyridin-8-yl)-5-pentylbenzene-1,3-diol;
5-Cyclopentyl-2-(7-methylimidazo[1,2-a]pyridin-8-yl)benzene-1,3-diol;
2-(2-trifluoromethyl-7-methylimidazo[1,2-a]pyridin-8-yl)-5-propylbenzene-1,3-diol;
2-(6-cyclopropyl-7-methylimidazo[1,2-a]pyridin-8-yl)-5-propylbenzene-1,3-diol;
2-(6-trifluoromethyl-7-methylimidazo[1,2-a]pyridin-8-yl)-5-propylbenzene-1,3-diol;
2-(6-chloro-7-methylimidazo[1,2-a]pyridin-8-yl)-5-pentylbenzene-1,3-diol;
2-(7-Methylimidazo[1,2-a]pyridin-8-yl)benzene-1,3-diol;
2-(7-methylimidazo[1,2-a]pyridin-8-yl)-5-(tert-pentyl)benzene-1,3-diol;
2-(6-chloro-7-methylimidazo[1,2-a]pyridin-8-yl)-5-(tert-pentyl)benzene-1,3-diol;
5-Chloro-2-(6-chloro-7-methylimidazo[1,2-a]pyridin-8-yl)benzene-1,3-diol;
2-(6-Fluoro-7-methylimidazo[1,2-a]pyridin-8-yl)-5-(3,3,3-trifluoropropyl)benzene-1,3-diol; and
2-(6-Chloroimidazo[1,2-a]pyridin-8-yl)-5-propylbenzene-1,3-diol.

In an embodiment, the compound of formula (I) is selected from the following compounds and salts thereof:
2-(7-methylimidazo[1,2-a]pyridin-8-yl)-5-propylbenzene-1,3-diol;
2-(6-chloro-7-methylimidazo[1,2-a]pyridin-8-yl)-5-propylbenzene-1,3-diol; and
2-(6-fluoro-7-methylimidazo[1,2-a]pyridin-8-yl)-5-propylbenzene-1,3-diol;

### Salts

In some embodiments, the compounds of formula (I) are provided in free base form.

Alternatively, it may be convenient or desirable to prepare, purify, and/or handle a corresponding salt of the compound, for example, a pharmaceutically-acceptable salt. Examples of pharmaceutically acceptable salts are discussed in “Pharmaceutical Salts: Properties, Selection, and Use”, 2^{nd} Edition, 2002, Stahl and Wermuth (Eds), Wiley-VCH, Weinheim, Germany.

Accordingly, in some embodiments the compounds of formula (I) are provided as salts, for example in a protonated form together with a suitable counter anion.

Suitable counter anions include both organic and inorganic anions. Example of suitable inorganic anions include those derived from inorganic acids, including chloride (Cl⁻), bromide (Br⁻), iodide (I⁻), sulfate (SO₄²⁻), sulfite (SO₃²⁻), nitrate (NO₃⁻), nitrite (NO₂⁻), phosphate (PO₄³⁻), and phosphite (PO₃³⁻). Examples of suitable organic anions include 2-acetoxybenzoate, acetate, ascorbate, aspartate, benzoate, camphorsulfonate, cinnamate, citrate, edetate, ethanedisulfonate, ethanesulfonate, formate, fumarate, gluconate, glutamate, glycolate, hydroxymalate, carboxylate, lactate, laurate, lactate, maleate, malate, methanesulfonate, oleate, oxalate, palmitate, phenylacetate, phenylsulfonate, propionate, pyruvate, salicylate, stearate, succinate, sulfanilate, tartarate, toluenesulfonate, and valerate. Examples of suitable polymeric organic anions include those derived from tannic acid and carboxymethyl cellulose. In an embodiment, the counter anion is chloride or formate, such as formate.

Alternatively, in some embodiments the compounds of formula (I) are provided as salts, for example in a deprotonated form together with a suitable counter cation.

Suitable counter cations include both organic and inorganic cations. Examples of suitable inorganic cations include alkali metal ions such as Na⁺ and K⁺, alkaline earth cations such as Ca²⁺ and Mg²⁺, and other cations such as Al³⁺. Examples of suitable organic cations include the ammonium ion (i.e., NH₄⁺) and substituted ammonium ions (e.g., NH₃R+, NH₂R₂⁺, NHR₃⁺, NR₄⁺). Examples of substituted ammonium ions include those derived from ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

In a preferred embodiment there is provided a compound of formula (I), or a pharmaceutically-acceptable salt thereof.

### Solvates

In some embodiments, the compounds of formula (I) are provided in desolvated form, for example, in dehydrated form.

Alternatively, it may be convenient or desirable to prepare, purify, and/or handle a corresponding solvate of the compound.

Accordingly, in some embodiments the compounds of formula (I) are provided in the form of a solvate (a complex of solute (e.g., compound, salt of compound) and solvent). Examples of solvates include hydrates, for example, a mono-hydrate, a di-hydrate and a tri-hydrate.

### N-Oxides

Where compounds of formula (I) contain an sp² nitrogen atom (—N═), for example in a heteroaryl group, it may be convenient to prepare, purify, and/or handle the corresponding N-oxide (—N(→O)═), also denoted as (—N⁺(O⁻)═).

Accordingly, in some embodiments certain compounds of formula (I) are provided in the form of an N-oxide. For example, pyridine may be substituted to give pyridine N-oxide.

### Certain Isomers

Certain compounds of formula (I) may exist in one or more particular optical, enantiomeric, diastereomeric, epimeric, stereoisomeric, tautomeric, or conformational forms, including but not limited to, D- and L-forms; d- and l-forms; (+) and (−) forms; syn- and anti-forms; axial and equatorial forms; boat-, chair-, twist boat-, envelope-, and half chair-forms; and combinations thereof, hereinafter collectively referred to as “isomers” or “isomeric forms”.

Specifically excluded from the term “isomers,” as used herein, are structural (or constitutional) isomers (i.e., isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference 2-pyridinyl is not to be construed as a reference to its structural isomer, 3-pyridinyl.

The above exclusion does not pertain to tautomeric forms, for example, keto-, enol-, and enolate-forms, as in, for example, the following tautomeric pairs: keto/enol, imine/enamine, amide/imino alcohol, nitroso/oxime, and lactam/lactim.

Included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including (wholly or partially) racemic and other mixtures thereof.

### Methods of Synthesis

Methods for the synthesis of specific example compounds within the scope of formula (I) are set out in the worked examples.

The invention provides a first method of preparing a compound of formula (I), the method comprising:
(1a) reacting a compound of formula (II) with a compound of formula (III) to give a compound of formula (IV):

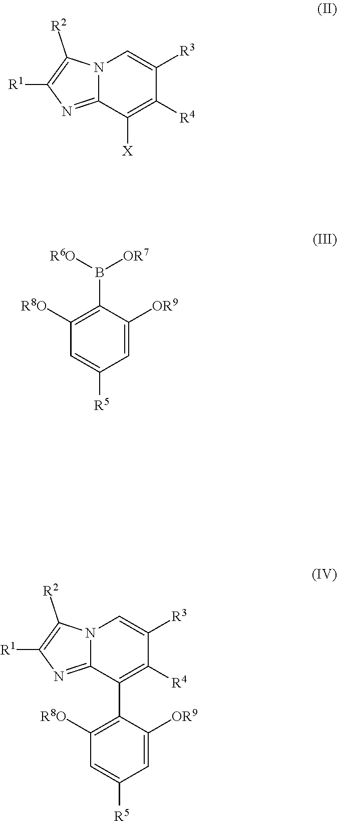

where:
R¹, R², R³, R⁴, and R⁵ are as defined herein; X is chloro, bromo, iodo, or triflate;
R⁶ and R⁷ are hydrogen, alkyl or phenyl; or R⁶ and R⁷ are joined to form a cyclic boronic ester (such as pinacol, neopentyl or catechol boronic esters); and
R⁸ and R⁹ are suitable protecting groups such as methyl or benzyl; and
(1b) converting a compound of formula (IV) to a compound of formula (I).

In a preferred embodiment, X is bromo or iodo. Most preferably X is bromo.

In a preferred embodiment, R⁶ and R⁷ together form —C(Me)₂C(Me)₂- (a pinacol boronic ester). In a preferred embodiment, R⁶ and R⁷ are both hydrogen.

In an embodiment R⁸ and R⁹ are alcohol protecting groups selected from methyl, methoxymethyl, 2-(trimethylsilyl) ethoxymethyl, trimethylsilyl, tert-butyldimethylsilyl, triisopropylsilyl, benzyl, and 4-methoxybenzyl. In a preferred embodiment, R⁸ and R⁹ are both methyl. In a preferred embodiment, R⁸ and R⁹ are both benzyl.

Preferably, step (1a) comprises reacting a compound of formula (II) with a compound of formula (III) and a palladium catalyst. Suitable palladium catalysts include Pd(dppf)Cl₂, X-Phos-Pd-G3 ((2-Dicyclohexylphosphino-2′,4′,6′-triisopropyl-1,1′-biphenyl)[2-(2′-amino-1,1′-biphenyl)]palladium(II) methanesulfonate), SPhos-Pd-G2 (Chloro(2-dicyclohexylphosphino-2′,6′-dimethoxy-1,1′-biphenyl)[2-(2′-amino-1,1′-biphenyl)]palladium(II)), cataCXium® A Pd G3 ([(Di(1-adamantyl)-butylphosphine)-2-(2′-amino-1,1′-biphenyl)]palladium(II) methanesulfonate), APhos-Pd-G3 ([4-(Di-tert-butylphosphino)-N,N-dimethylaniline-2-(2′-aminobiphenyl)]palladium(II) methanesulfonate), P(Cy₃)-Pd-G3 ([(Tricyclohexylphosphine)-2-(2′-aminobiphenyl)]palladium(II) methanesulfonate), and PEPPSI-IPENT (Dichloro[1,3-bis(2,6-Di-3-pentylphenyl) imidazol-2-ylidene](3-chloropyridyl)palladium(II)). Preferred palladium catalysts include Pd(dppf)Cl₂ and SPhos-Pd-G2 (Chloro(2-dicyclohexylphosphino-2′,6′-dimethoxy-1,1′-biphenyl)[2-(2′-amino-1,1′-biphenyl)]palladium(II)).

Preferably, step (1a) further comprises reacting a compound of formula (II) with a compound of formula (III) and a base. Suitable bases include sodium carbonate (Na₂CO₃), potassium carbonate (K₂CO₃), and caesium carbonate (Cs₂CO₃).

Typically, step (1a) is carried out in a solvent. Suitable solvents include dioxane, tetrahydrofuran (THF), dimethylformamide (DMF), cyclopentyl methyl ether (CPME), 1,2-dimethoxyethane (DME), dimethylacetamide (DMA), toluene, ethanol, propanol, isopropanol, butan-1-ol, butan-2-ol, pentanol, water, and mixtures thereof. Preferred solvents include dioxane, tetrahydrofuran (THF), dimethylformamide (DMF), cyclopentyl methyl ether (CPME), water, and mixtures thereof.

Optionally, certain additives may be used in step (1a). Suitable additives include caesium fluoride (CsF).

Step (1a) is typically performed at elevated temperature (above ambient temperature; approximately 20 °C). Methods for providing heat during the reaction are known and include, for example, using a reaction vessel having an external heating jacket or using microwave heating.

Typically, step (1a) comprises reacting a compound of formula (II) with a compound of formula (III) at a temperature of from 60 °C to 140 °C, preferably 80 °C to 140 °C, more preferably 80 °C to 120 °C.

Step (1a) may be performed for sufficient time to allow a desired quantity of the coupling product to form. Typically, the step (1a) is performed until substantially all of the compound of formula (II) has been consumed.

Typically, the step (1a) comprises reacting a compound of formula (II) with a compound of formula (III) for 1 hour to 24 hours.

Step (1b) comprises reacting a compound of formula (IV) under conditions suitable for removal of the R⁸ and R⁹ protecting groups. A skilled person will be able to determine suitable deprotection conditions, depending on the nature of the R⁸ and R⁹ protecting groups and guided by the examples described below.

In a preferred embodiment, R⁸ and R⁹ are both methyl and step (1b) comprises reacting a compound of formula (IV) with a Lewis acid, such as BBr₃, AlCl₃, BeCl₂, trimethylsilyl iodide, or pyridine hydrochloride. Preferably, R⁸ and R⁹ are both methyl and step (1b) comprises reacting a compound of formula (IV) with BBr₃. Typically, step (1b) is carried out in a solvent. Suitable solvents include dichloromethane (DCM). Typically, step (1b) is performed at a temperature of from 0 °C to 30 °C, preferably 0 °C to 20 °C.

In a preferred embodiment, R⁸ and R⁹ are both benzyl and step (1b) comprises reacting a compound of formula (IV) with hydrogen in the presence of a suitable catalyst (such as palladium on carbon, or platinum on carbon) and a suitable solvent (such as methanol, ethanol, acetonitrile, or tetrahydrofuran (THF)).

The invention also provides a compound obtained or obtainable by the method set out in paragraphs [0059] to [0073] above.

### Intermediates

The invention provides intermediates useful in the preparation of a compound formula (I). An intermediate of the invention is a compound of formula (II):

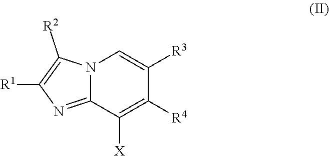

wherein R¹ is hydrogen, C₁₋₃alkyl, C₁₋₃alkoxy, C₁₋₃haloalkyl, or halo;
R² is hydrogen, C₁₋₃alkyl, C₁₋₃alkoxy, or halo;
R³ is hydrogen, C₁₋₃alkyl, C₁₋₃alkoxy, C₁₋₃haloalkyl, C₃₋₆cycloalkyl, or halo;
R⁴ is hydrogen, C₁₋₃alkyl, C₁₋₃alkoxy, C₁₋₃haloalkyl, C₃₋₆cycloalkyl, or halo; and
X is chloro, bromo, iodo, or triflate.

In an embodiment there is provided a compound of formula (II), wherein R¹ is hydrogen, methyl, CF₃, or chloro. In an embodiment there is provided a compound of formula (II), wherein R² is hydrogen, methyl, or chloro. In an embodiment there is provided a compound of formula (II), wherein R³ is hydrogen, methyl, CF₃, cyclopropyl, fluoro, or chloro. In an embodiment there is provided a compound of formula (II), wherein R⁴ is methyl, CF₃, cyclopropyl, fluoro, or chloro. In an embodiment there is provided a compound of formula (II), wherein X is bromo.

In an embodiment, there is provided a compound of formula (II) selected from:
8-Bromo-7-methylimidazo[1,2-a]pyridine;
8-Bromo-2,7-dimethylimidazo[1,2-a]pyridine;
8-Bromo-6-chloro-7-methylimidazo[1,2-a]pyridine;
8-Chloro-7-(trifluoromethyl) imidazo[1,2-a]pyridine;
8-Bromo-6,7-dimethylimidazo[1,2-a]pyridine;
8-Bromo-7-fluoroimidazo[1,2-a]pyridine;
8-Bromo-3,7-dimethylimidazo[1,2-a]pyridine;
8-Bromo-7-cyclopropylimidazo[1,2-a]pyridine;
8-Bromo-3-chloro-7-methylimidazo[1,2-a]pyridine;
8-Bromo-6-fluoro-7-methylimidazo[1,2-a]pyridine;
8-Bromo-2-chloro-7-methylimidazo[1,2-a]pyridine;
8-Bromo-2-(trifluoromethyl)-7-methylimidazo[1,2-a]pyridine; and
8-Bromo-6-(trifluoromethyl)-7-methylimidazo[1,2-a]pyridine.

A further intermediate of the invention is a compound of formula (IV):

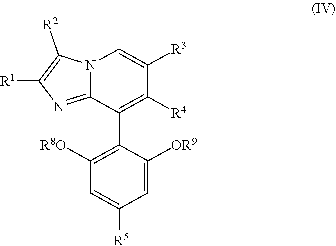

wherein R¹ is hydrogen, C₁₋₃alkyl, C₁₋₃alkoxy, C₁₋₃haloalkyl, or halo;
R² is hydrogen, C₁₋₃alkyl, C₁₋₃alkoxy, or halo;
R³ is hydrogen, C₁₋₃alkyl, C₁₋₃alkoxy, C₁₋₃haloalkyl, C₃₋₆cycloalkyl, or halo;
R⁴ is hydrogen, C₁₋₃alkyl, C₁₋₃alkoxy, C₁₋₃haloalkyl, C₃₋₆cycloalkyl, or halo;
R⁵ is hydrogen, C₁₋₁₀alkyl, C₁₋₁₀haloalkyl, C₃₋₆cycloalkyl, or halo; and
R⁸ and R⁹ are suitable protecting groups such as methyl or benzyl.

In an embodiment there is provided a compound of formula (IV), wherein R¹ is hydrogen, methyl, CF₃, or chloro. In an embodiment there is provided a compound of formula (IV), wherein R² is hydrogen, methyl, or chloro. In an embodiment there is provided a compound of formula (IV), wherein R³ is hydrogen, methyl, CF₃, cyclopropyl, fluoro, or chloro. In an embodiment there is provided a compound of formula (IV), wherein R⁴ is methyl, CF₃, cyclopropyl, fluoro, or chloro. In an embodiment there is provided a compound of formula (IV), wherein R⁵ is hydrogen, C₃₋₅alkyl, C₃₋₅haloalkyl, cyclopentyl, or chloro. In an embodiment there is provided a compound of formula (IV), wherein R⁵ is hydrogen, propyl, pentyl, 1,1-dimethylpropyl (tert-pentyl), 3,3,3-trifluoropropyl, cyclopentyl, or chloro. In an embodiment there is provided a compound of formula (IV), wherein R⁸ and R⁹ are methyl. In an embodiment there is provided a compound of formula (IV), wherein R⁸ and R⁹ are benzyl.

In an embodiment, there is provided a compound of formula (IV) selected from:
8-(2,6-Dimethoxy-4-propylphenyl) imidazo[1,2-a]pyridine;
8-(2,6-Dimethoxy-4-propylphenyl)-7-methylimidazo[1,2-a]pyridine;
8-(2,6-Dimethoxy-4-propylphenyl)-2,7-dimethylimidazo[1,2-a]pyridine;
6-Chloro-8-(2,6-dimethoxy-4-propylphenyl)-7-methylimidazo[1,2-a]pyridine;
8-(2,6-Dimethoxy-4-propylphenyl)-7-trifluoromethylimidazo[1,2-a]pyridine;
7-Chloro-8-(2,6-dimethoxy-4-propylphenyl) imidazo[1,2-a]pyridine;
8-(2,6-Dimethoxy-4-propylphenyl)-6,7-dimethylimidazo[1,2-a]pyridine;
7-Fluoro-8-(2,6-dimethoxy-4-propylphenyl) imidazo[1,2-a]pyridine;
8-(2,6-Dimethoxy-4-propylphenyl)-3,7-dimethylimidazo[1,2-a]pyridine;
3-Chloro-8-(2,6-dimethoxy-4-propylphenyl)-7-methylimidazo[1,2-a]pyridine;
6-Fluoro-8-(2,6-dimethoxy-4-propylphenyl)-7-methylimidazo[1,2-a]pyridine;
2-Chloro-8-(2,6-dimethoxy-4-propylphenyl)-7-methylimidazo[1,2-a]pyridine;
8-(2,6-Dimethoxy-4-pentylphenyl) 7-methylimidazo[1,2-a]pyridine;
8-(2,6-Dimethoxyphenyl)-7-methylimidazo[1,2-a]pyridine;
8-(2,6-Dimethoxy-4-propylphenyl)-2-trifluoromethyl-7-methylimidazo[1,2-a]pyridine;
8-(2,6-Dimethoxy-4-propylphenyl)-6-trifluoromethyl-7-methylimidazo[1,2-a]pyridine;
6-Chloro-8-(2,6-dimethoxy-4-pentylphenyl)-7-methylimidazo[1,2-a]pyridine;
8-(2,6-Dimethoxy-4-propylphenyl)-7-cyclopropylimidazo[1,2-a]pyridine;
6-Chloro-8-(4-chloro-2,6-dimethoxyphenyl)-7-methylimidazo[1,2-a]pyridine;
6-Chloro-8-(2,6-dimethoxy-4-tert-pentylphenyl)-7-methylimidazo[1,2-a]pyridine;
8-(2,6-dimethoxy-4-tert-pentylphenyl)-7-methylimidazo[1,2-a]pyridine;
8-(4-Cyclopentyl-2,6-dimethoxyphenyl)-7-methylimidazo[1,2-a]pyridine;
6-Cyclopropyl-8-(2,6-dimethoxy-4-propylphenyl)-7-methylimidazo[1,2-a]pyridine;
8-(2,6-Dimethoxy-4-(3,3,3-trifluoropropyl)phenyl)-6-fluoro-7-methylimidazo[1,2-a]pyridine;
and
6-Chloro-8-(2,6-dimethoxy-4-propylphenyl) imidazo[1,2-a]pyridine.

### Pharmaceutical Compositions

While it is possible for the compounds of formula (I) to be administered alone, it is preferable to administer a pharmaceutical composition (e.g., a formulation, preparation, or medicament) comprising a compound of formula (I) together with one or more other pharmaceutically acceptable ingredients.

Accordingly, the invention provides a pharmaceutical composition comprising a compound of formula (I), or a salt thereof, together with one or more pharmaceutically acceptable ingredients.

Suitable pharmaceutically acceptable ingredients (e.g. carriers, diluents, excipients, etc.) can be found in standard pharmaceutical texts, for example, Remington: The Science and Practice of Pharmacy, 20th Edition, 2000, pub. Lippincott, Williams & Wilkins; and Handbook of Pharmaceutical Excipients, 9th edition, 2020, pub. Pharmaceutical Press.

Examples of suitable pharmaceutically acceptable ingredients include pharmaceutically acceptable carriers, diluents, excipients, adjuvants, fillers, buffers, binders, disintegrants, preservatives, antioxidants, lubricants, stabilisers, solubilisers, surfactants (e.g., wetting agents), masking agents, colouring agents, flavouring agents, and sweetening agents.

The pharmaceutical composition may be in any suitable form. Examples of suitable forms include liquids, solutions (e.g., aqueous, nonaqueous), suspensions (e.g., aqueous, non-aqueous), emulsions (e.g., oil-in-water, water-in-oil), syrups, electuaries, mouthwashes, drops, tablets (including, e.g., coated tablets), granules, powders, lozenges, pastilles, capsules (including, e.g., hard and soft gelatin capsules), cachets, pills, ampoules, boluses, suppositories, pessaries, tinctures, gels, pastes, ointments, creams, lotions, oils, foams, sprays, and aerosols.

### Medical Treatment

The inventors have found that the compounds of formula (I) are biologically active. The worked examples demonstrate that compounds of formula (I) display anticonvulsant activity in rodent models of generalised seizure. As such, compounds of formula (I), and their salts, as well as pharmaceutical compositions comprising compounds of formula (I), or their salts, will be useful in medical treatment.

Accordingly, the invention provides a compound of formula (I), or a salt thereof, for use in a method of treatment, for example for use in a method of treatment of the human or animal body by therapy (i.e. a method of therapy).

The invention also provides a compound of formula (I), or a salt thereof, for use as a medicament.

The invention also provides a method of treatment comprising administering to a subject in need of treatment a therapeutically effective amount of a compound of formula (I), or a salt thereof.

The invention also provides the use of compound of formula (I), or a salt thereof, for the manufacture of a medicament.

The invention also provides the use of a compound of formula (I), or a salt thereof, as a medicament.

The invention also provides the use of a compound of formula (I), or a salt thereof, in a method of treatment.

### Conditions Treated

The inventors have found that the compounds of formula (I) display anticonvulsant activity in rodent models of generalised seizure. Accordingly, the compounds of formula (I), or their salts, as well as pharmaceutical compositions comprising the compounds of formula (I), or their salts, will be useful in the treatment of certain conditions associated with seizure.

Similarly, the compounds of formula (I), or their salts, as well as pharmaceutical compositions comprising the compounds of formula (I) or their salts, will be useful as medicaments for treating (and in the manufacture of medicaments for treating) certain conditions associated with seizure.

### Seizure Types

In an embodiment, the condition associated with seizure is selected from focal onset seizure (focal seizure), generalised onset seizure (generalised seizure), and seizure with unknown onset (unknown onset seizure).

Focal onset seizures may be further characterised by the level of awareness. In an embodiment, the focal onset seizure is focal onset seizure with awareness. In an embodiment, the focal onset seizure is a focal onset seizure with impaired awareness (with impairment).

Focal onset seizures may be further characterised by motor onset or non-motor onset features. In an embodiment, the focal onset seizure is a focal onset seizure with a motor onset feature, such as a motor onset feature selected from automatisms, atonic, clonic, epileptic spasms, hyperkinetic, myoclonic and tonic. In an embodiment, the focal onset seizure is a focal onset seizure with a non-motor onset feature, such as a nonmotor onset feature selected from autonomic, behaviour arrest, cognitive, emotional and sensory.

Focal onset seizures may also present with focal to bilateral tonic-clonic propagation. In an embodiment, the focal onset seizure is a focal to bilateral tonic-clonic seizure.

Generalised onset seizures may be characterised by motor or non-motor (absence) features. In an embodiment, the generalised onset seizure is a generalised onset seizure with a motor feature, such as a motor feature selected from tonic-clonic, clonic, tonic, myoclonic, myoclonic-tonic-clonic, myoclonic-atonic, atonic, and epileptic spasms. In an embodiment, the generalised onset seizure is a generalised onset seizure with a non-motor (absence) feature, such as a non-motor (absence) feature selected from typical, atypical, myoclonic and eyelid myoclonia.

Seizures with unknown onset may be characterised by motor or non-motor features. In an embodiment, the unknown onset seizure is an unknown onset seizure with a motor feature, such as a motor feature selected from tonic-clonic and epileptic spasms. In an embodiment, the unknown onset seizure is an unknown onset seizure with a non-motor (absence) feature, such as behavioural arrest.

In an embodiment, the condition associated with seizure is a seizure type that may be present in one or more of focal onset, generalised onset or unknown onset seizures, such as a seizure type selected from typical absence seizure, atypical absence seizure, atonic seizure, clonic seizure, tonic seizure, tonic-clonic seizure, febrile seizure, focal to bilateral tonic clonic seizure, gelastic and dacrystic seizure, myoclonic seizure, myoclonic-tonic-clonic seizure, myoclonic-atonic seizure, and epileptic (or infantile) spasms.

### Epilepsy Types and Epilepsy Syndromes

In an embodiment, the condition associated with seizure is an epilepsy, such as an epilepsy selected from focal epilepsy, generalised epilepsy, and combined generalised & focal epilepsy.

In an embodiment, the condition associated with seizure is an epilepsy syndrome, such as an epilepsy syndrome selected from syndromes with onset in neonates and infancy, syndromes with onset in childhood, syndromes with onset at a variable age, and idiopathic generalised epilepsy syndromes (IGEs).

In an embodiment the condition associated with seizure is an epilepsy syndrome with onset in neonates and infancy, such as an epilepsy syndrome with onset in neonates and infancy selected from self-limited epilepsies (such as self-limited neonatal epilepsy, self-limited familial neonatal-infantile epilepsy, self-limited infantile epilepsy, genetic epilepsy with febrile seizures plus, or myoclonic epilepsy in infancy), developmental and epileptic encephalopathies—DEEs (such as early infantile developmental and epileptic encephalopathy (EIDEE), epilepsy in infancy with migrating focal seizures (EIMFS), infantile epileptic spasms syndrome (West Syndrome), or Dravet syndrome), and aetiology-specific syndromes (such as KCNQ2-DEE, pyridoxine-dependent DEE, pyridox(am)ine 5′-phosphate deficiency DEE, CDKL5-DEE, PCDH19 clustering epilepsy, glucose transporter 1 deficiency syndrome, gelastic seizures with hypothalamic hamartoma, or Sturge Weber syndrome)—further information is provided in the aetiology section below.

In an embodiment the condition associated with seizure is an epilepsy syndrome with onset in childhood, such as an epilepsy syndrome with onset in childhood selected from self-limited focal epilepsies of childhood (such as childhood occipital visual epilepsy (COVE), self-limited epilepsy with autonomic seizures, self-limited epilepsy with centrotemporal spikes, or photosensitive occipital lobe epilepsy (POLE)), DEEs with onset in childhood (such as epilepsy with myoclonic atonic seizures, Lennox Gastaut syndrome, febrile infection-related epilepsy syndrome (FIRES), hemiconvulsion-hemiplegia-epilepsy syndrome, developmental and epileptic encephalopathy with spike-and-wave activation in sleep (DEE-SWAS), epileptic encephalopathy with spike-and-wave activation in sleep (EE-SWAS), or Landau-Kleffner syndrome), and genetic generalised epilepsies of childhood (such as epilepsy with eyelid myoclonia, or epilepsy with myoclonic absence).

In an embodiment the condition associated with seizure is an epilepsy syndrome with onset at a variable age, such as an epilepsy syndrome with onset at variable age selected from juvenile myoclonic epilepsy (JME), juvenile absence epilepsy (JAE), epilepsy with generalised tonic-clonic seizures alone (GTCA), childhood occipital visual epilepsy (COVE), photosensitive occipital lobe epilepsy (POLE), familial mesial temporal lobe epilepsy (FMTLE), epilepsy with auditory features (EAF), mesial temporal lobe epilepsy with hippocampal sclerosis (MTLE-HS), sleep-related hypermotor/hyperkinetic epilepsy (SHE), familial focal epilepsy with variable foci (FFEVF), epilepsy with reading induced seizures (EwRIS), progressive myoclonus epilepsies (PME), febrile-infection related epilepsy syndrome (FIRES) and Rasmussen syndrome.

In an embodiment the condition associated with seizure is an idiopathic generalised epilepsy syndrome (IGE), such as an IGE selected from childhood absence epilepsy (CAE), juvenile absence epilepsy (JAE), juvenile myoclonic epilepsy (JME), and epilepsy with generalised tonic-clonic seizures alone (GTCA).

### Aetiology

In an embodiment, the condition associated with seizure is an epilepsy defined by aetiology (cause), such as an epilepsy having an aetiology selected from structural, genetic, infection, metabolic, and autoimmune.

In an embodiment, the condition associated with seizure is an epilepsy having a structural aetiology (“structural epilepsy”), such as a structural aetiology selected from focal cortical dysplasia (FCD), hypothalamic hamartoma (HH), hypoxic ischemic encephalopathy (HIE), incontinent pigment (IP), mesial temporal sclerosis (MTS), neurofibromatosis type 1, multiple sclerosis (MS), encephalotrigeminal angiomatosis (Sturge Weber syndrome; SWS), traumatic brain injury (TBI); periventricular nodular heterotopia (PVNH), polymicrogyria (PMG), tuberous sclerosis complex (TSC), and brain tumours.

In an embodiment, the condition associated with seizure is an epilepsy having a genetic aetiology (“genetic epilepsy”), such as a genetic aetiology selected from Angelman Syndrome, PCDH19 mutation, ring chromosome 20 syndrome (RC20), CACNA1A mutation, CDKL5 deficiency disorder, GRIN2A mutation, SCN8A mutation, SLC2A1 (Glut1 deficiency syndrome), TBCK mutation, Rett syndrome (MECP2 mutation), SYNGAP1 mutation, KCNQ2 mutation, STXBP1 mutation, SCN1A mutation, CHD2 mutation, PRRT2 mutation, SLC6A1 mutation, and GATOR1-related epilepsy.

In an embodiment, the condition associated with seizure is an epilepsy having an infection aetiology (“infection epilepsy”), such as an infection aetiology selected from neurocysticercosis, cerebral malaria, TORCH infections (toxoplasmosis, other agents, rubella/German measles, cytomegalovirus, and herpes simplex), bacterial meningitis, viral encephalitis, tuberculosis, and human immunodeficiency virus (HIV).

In an embodiment, the condition associated with seizure is an epilepsy having a metabolic aetiology (“metabolic epilepsy”), such as a metabolic aetiology selected from glucose transporter type 1 (Glut 1) deficiency syndrome (SLC2A1), vitamin dependent metabolic disorders (such as pyridoxine, P5P or folinic acid dependent disorders), creatine transporter disorders, mitochondrial disorders, and storage disorders.

In an embodiment, the condition associated with seizure is an epilepsy having an autoimmune aetiology (“autoimmune epilepsy”), such as an autoimmune aetiology selected from Rasmussen syndrome, antiNMDA receptor encephalitis, limbic encephalitis (e.g. LGI1 antibody, or CASPR2 antibody related limbic encephalitis), and GAD65 antibody related epilepsy.

### Subject/Patient

The method of treatment typically comprises administering a compound of formula (I), or a salt thereof, to a subject or patient.

The subject/patient may be a chordate, a vertebrate, a mammal, a placental mammal, a marsupial (e.g., kangaroo, wombat), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orang-utan, gibbon), or a human.

The subject/patient may be any of its forms of development, for example, the subject/patient may be a neonate, an infant, a child, or an adult.

In a preferred embodiment, the subject/patient is a human, more preferably an adult human.

The subject/patient may also be a non-human mammal used in laboratory research, such as a rodent. Rodents include rats, mice, guinea pigs and chinchillas.

### Routes of Administration

The method of treatment may comprise administering a compound of formula (I), or a salt thereof, to a subject by any convenient route of administration, whether systemically/peripherally or topically (i.e., at the site of desired action).

The route of administration may be oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eyedrops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection or infusion, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; or by implant of a depot or reservoir, for example, subcutaneously or intramuscularly.

### Dosages

The method of treatment typically comprises administering a therapeutically effective amount of a compound of formula (I), or a salt thereof, to a subject.

Appropriate dosages of the compounds of formula (I), their salts, as well as pharmaceutical compositions comprising the compounds of formula (I), or their salts, can vary from patient to patient. Determining the optimal dosage will generally involve balancing the level of therapeutic benefit against any risk or deleterious side effects. The selected dosage level will depend on a variety of factors including, but not limited to, the activity of the particular compound of formula (I), the route of administration, the time of administration, the rate of excretion of the compound, the duration of the treatment, other active agents, compounds, and/or materials used in combination, the severity of the condition, and the species, sex, age, weight, condition, general health, and prior medical history of the patient. The dosage and route of administration will ultimately be at the discretion of the clinician, although generally the dosage will be selected to achieve local concentrations at the site of action which achieve the desired effect without causing substantial harmful or deleterious side-effects.

Administration can be effected in one dose, continuously or intermittently (e.g., in divided doses at appropriate intervals) throughout the course of treatment. Single or multiple administrations can be carried out with the dose level and pattern being selected by the treating clinician.

### Other Aspects and Embodiments

Each and every compatible combination of the embodiments described above is explicitly discloses herein, as if each and every combination was individually and explicitly recited.

Carious further aspects and embodiment of the present invention will be apparent to those skilled in the arti in view of the present disclosure.

Where used, “and/or” is to be taken as a specific disclosure of each of the relevant components or features alone as well as a specific disclosure of the combination of the components or features. For example, “A and/or B” is to be taken as specific disclosure of each of i) A, ii) B, and iii) A and B, just as if each were set out individually.

Where used, “selected from” a list of members is to be taken as a specific disclosure of the relevant members alone. For example, “selected from A, B, and C” is to be taken as a specific disclosure of each of i) A, ii) B, and iii) C, just as if each were set out individually.

Where used, “one or more selected from” a list of members is to be taken as a specific disclosure of the relevant members alone as well as a specific disclosure of the combination of the members. For example, “one or more selected from A, B, and C” is to be taken as specific disclosure of each of i) A, ii) B, iii) C, iv) A and B, v) A and C, vi) B and C, and vi) A and B and C, just as if each were set out individually.

Unless context dictates otherwise, the descriptions and definitions of the features set out above are not limited to any particular aspect or embodiment of the invention and apply equally to all aspects and embodiments which are described.

### DEFINITIONS

The following definitions are provided in order to aid understanding of the invention.

An alkyl group is a monovalent saturated hydrocarbon group. The alkyl group may be a C₁₋₆ alkyl group, for example a C₁₋₄, C₁₋₃ or a C₁₋₂ alkyl group. In this context, the prefix (e.g. C₁₋₆) denotes the number of carbon atoms in the hydrocarbon backbone. The alkyl group may be linear or branched.

Examples of C₁₋₆ linear alkyl groups include methyl (-Me), ethyl (-Et), n-propyl (-nPr), n-butyl (-nBu), n-pentyl (-Amyl) and n-hexyl.

Examples of C₁₋₆ branched alkyl groups include iso-propyl (-iPr), iso-butyl (-iBu), sec-butyl (-sBu), tert-butyl (-tBu), iso-pentyl, sec-pentyl, tert-pentyl, neo-pentyl, iso-hexyl, sec-hexyl, tert-hexyl and neo-hexyl.

The term “alkoxy” includes both straight and branched chain alkyl groups singularly bonded to oxygen. For example, “C₁₋₃alkoxy” includes methoxy, ethoxy, and iso-propoxy.

The term “haloalkyl” is used herein to refer to an alkyl group respectively in which one or more hydrogen atoms have been replaced by halogen (e.g. fluorine) atoms. Examples of C₁₋₃haloalkyl groups include fluoroalkyl groups such as —CHF₂, —CH₂CF₃, and perfluoroalkyl groups such as —CF₃, or —CF₂CF₃.

“Cycloalkyl” means a hydrocarbon monocyclic or bicyclic ring containing carbon atoms. Examples of C₃₋₆cycloalkyl groups include cyclopropyl, cyclobutyl, cyclopentyl, and cyclohexyl.

The term “halo” refers to fluoro, chloro, bromo and iodo.

Epilepsy is considered to be a disease of the brain defined by any of the following conditions: (1) At least two unprovoked (or reflex) seizures occurring >24 h apart; (2) one unprovoked (or reflex) seizure and a probability of further seizures similar to the general recurrence risk (at least 60%) after two unprovoked seizures, occurring over the next 10 years; (3) diagnosis of an epilepsy syndrome (A practical clinical definition of epilepsy by the International League Against Epilepsy (ILAE), 2014).

The term “generalised seizure” (“generalised onset seizures”) refers to seizures conceptualized as originating at some point within the brain and rapidly engaging bilaterally distributed networks (Operational Classification of Seizure Types by the ILAE, 2017).

The term “focal seizure” (“focal onset seizures”) refers to seizures originating within networks limited to one hemisphere of the brain. They may be discretely localized or more widely distributed. Focal seizures may originate in subcortical structures (Operational Classification of Seizure Types by the ILAE, 2017). When a subject is awake and aware during a seizure it's referred to as a focal aware seizure. When a subject is confused or their awareness is affected in some way during a focal seizure, it's referred to as a focal impaired awareness seizure.

The term “seizure with unknown onset” (“unknown onset seizures”) refers to seizures whose onset is missed (e.g. because the subject was asleep or alone) or obscured. These seizures may be later classified as focal onset or generalised onset when more information becomes available (Operational Classification of Seizure Types by the ILAE, 2017).

The term “pharmaceutically acceptable” pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each ingredient (e.g. carrier, diluent, excipient, etc.) must also be “acceptable” in the sense of being compatible with the other ingredients of the composition.

The term “therapeutically-effective amount” pertains to that amount of a compound, or a material, composition or dosage form comprising a compound, which is effective for producing some desired therapeutic effect, commensurate with a reasonable benefit/risk ratio, when administered in accordance with a desired treatment regimen.

A “tonic-clonic seizure” occurs in two phases, a tonic phase typically involving muscle stiffening and loss of consciousness, and a clonic phase typically involving rhythmically jerking of the limbs.

### Worked Examples

Certain aspects and embodiments of the invention will not be illustrated by way of example and with reference to the figures described above.

### Analytical Methods

### NMR

Bruker Avance 400 MHz, 5 mm QNP probe H, C, F, P, single Z gradient, two channel instrument running TopSpin 2.1

Bruker Avance III 400 MHz, 5 mm BBFO Plus probe, single Z gradient, two channel instrument running TopSpin 3.1.

### LCMS

| Instrumentation | UPLC + Waters DAD + Waters SQD2, single | | |
|---|---|---|---|
| | quadrapole UPLC-MS | | |
| Column | Acquity UPLC HSS C18 1.8 um 100 × 2.1 mm. | | |
| | (Plus guard cartridge), maintained at 40 °C | | |
| Mobile | Water (High purity via PureLab Option unit) | | |
| Phase A | with 0.1% formic acid | | |
| Mobile | Acetonitrile (Far UV grade) with 0.1% | | |
| Phase B | (V/V) formic acid | | |
| Flow | 0.4 ml/min | | |
| | Time (mins) | % A | % B |
| Gradient | 0.0 | 95 | 05 |
| Program | 0.4 | 95 | 05 |
| | 6.0 | 05 | 95 |
| | 6.8 | 05 | 95 |
| | 7.0 | 95 | 05 |
| | 8.0 | 95 | 05 |
| Sample | 0.5-2 ul (concentration ~0.2-1 mg/ml). | | |
| Detectors | UV, diode array 210 nm-400 nm Resolution | | |
| | 1.2 nm Other wavelength traces are extracted | | |
| | from the DAD data | | |
| | MS, mass 100-700 (or −1500 for HM method) | | |
| | in ES+ & ES− (300 μl/min split to MS) | | |
| Instrumentation | UPLC + Waters DAD + Waters SQD2, single | | |
| | quadrapole UPLC-MS | | |
| Column | Acquity UPLC BEH C18 1.7 um 100 × 2.1 mm. | | |
| | (Plus guard cartridge), maintained at 40 °C | | |
| Mobile | Water (High purity via PureLab Option unit) | | |
| Phase A | with 10 mM ammonium bicarbonate (ammonium | | |
| | hydrogen carbonate) | | |
| Mobile | Acetonitrile (Far UV grade) | | |
| Phase B | | | |
| Flow | 0.4 ml/min | | |
| | Time (mins) | % A | % B |
| Gradient | 0.0 | 95 | 05 |
| Program | 0.4 | 95 | 05 |
| | 6.0 | 05 | 95 |
| | 6.8 | 05 | 95 |
| | 7.0 | 95 | 05 |
| | 8.0 | 95 | 05 |

| | |
|---|---|
| Sample | 0.5-2 ul (concentration ~0.2-1 mg/ml). |
| Detectors | UV, diode array 210 nm-400 nm Resolution |
| | 1.2 nm Other wavelength traces are extracted |
| | from the DAD data |
| | MS, mass 100-700 (or −1500 for HM method) |
| | in ES+ & ES− (300 μl/min split to MS) |

### ABBREVIATIONS

| | |
|---|---|
| Bn | Benzyl |
| Bu | Butyl |
| CPME | Cyclopentyl Methyl Ether |
| DCM | Dichloromethane |
| DMF | N,N-Dimethylformamide |
| DMSO | Dimethylsulfoxide |
| dppf | diphenylphosphinoferrocene |
| EtOH | Ethanol |
| HPLC | High Performance Liquid Chromatography |
| LCMS | Liquid Chromatography Mass Spectrometry |
| Me | Methyl |
| NBS | N-BromosuCCinimide |
| NCS | N-ChlorosuCCinimide |
| NMP | N-Methyl Pyrrolidinone |
| NMR | Nuclear Magnetic Resonance |
| PMB | Para-methoxybenzyl |
| S Phos | Chloro(2-dicyclohexylphosphino-2′,6′- |
| Pd G2 | dimethoxy-1,1′-biphenyl)[2-(2′-amino- |
| | 1,1′-biphenyl)]palladium(II) - |
| | second generation SPhos Precatalyst |
| TFA | Trifluoroacetic acid |
| THF | Tetrahydrofuran |

### Example 1: Synthesis of Compounds

In general, the compounds of the invention can be prepared by Suzuki reaction of an appropriately protected dihydroxyphenylboronic acid with an appropriate 8-halo-imidazo[1,2-a]pyridine, followed by deprotection of the resulting intermediate.

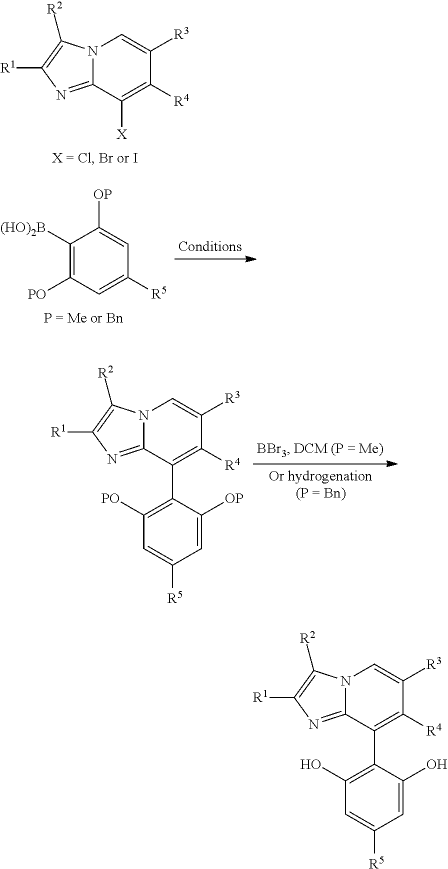

Aryl halide and boronic acid precursors can be purchased from commercial sources, prepared according to literature procedures or synthesised according to one of the following methods.

### Synthesis of Intermediates

### General Method A

General Method A is illustrated using the synthesis of Intermediate 1.

### Intermediate 1: (2,6-Dimethoxy-4-propyl-phenyl)boronic acid

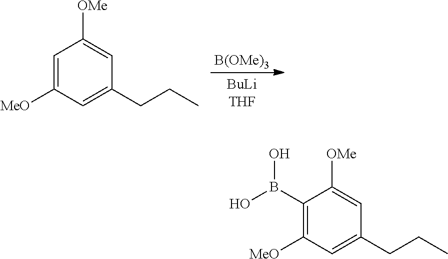 [SMILES: CCCc1cc(OC)cc(OC)c1 ; "B(OMe)3" ; CCCc1cc(OC)c(B(O)O)c(OC)c1]

1,3-Dimethoxy-5-propyl-benzene (10.00 g, 55.5 mmol) was stirred in tetrahydrofuran (230 mL) at 0 °C under an atmosphere of nitrogen and n-butyllithium solution (2.5 M in hexanes, 27 mL, 66.6 mmol) was added over 10 minutes. The reaction mixture was stirred for 1 hour at 0 °C then cooled to −78 °C, treated with trimethyl borate (19 mL, 166 mmol) and allowed to warm to room temperature over 150 minutes. The reaction mixture was quenched with saturated aqueous ammonium chloride solution (150 mL) and ethyl acetate (200 mL) was added. The layers were separated and the aqueous layer was extracted with ethyl acetate (150 mL). The combined organic layers were dried (magnesium sulfate) and concentrated in vacuo. The residue was purified by column chromatography on silica gel, eluting with 0-50% ethyl acetate in dichloromethane to give the title compound as a white solid (10.1 g, 81%). ¹H NMR (400 MHz, CDCl₃) δ 7.17 (s, 2H), 6.45 (s, 2H), 3.90 (s, 6H), 2.59 (t, J=7.7 Hz, 2H), 1.72-1.62 (m, 2H), 0.97 (t, J=7.3 Hz, 3H).

General Method A, using the appropriate dimethoxyarene, was also used to generate the following intermediate:

### Intermediate 2: (2,6-Dimethoxy-4-pentylphenyl)boronic acid

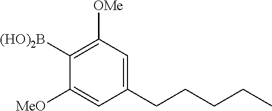 [SMILES: CCCCCc1cc(OC)c(B(O)O)c(OC)c1]

¹H NMR (400 MHz, CDCl₃) δ 7.16 (s, 2H), 6.45 (s, 2H), 3.90 (s, 6H), 2.60 (t, J=7.8 Hz, 2H), 1.68-1.58 (m, 2H), 1.39-1.30 (m, 4H), 0.91 (t, J=6.9 Hz, 3H).

### General Method B

General Method B is illustrated using the synthesis of Intermediate 3.

### Intermediate 3: (4-(tert-Pentyl)-2,6-dimethoxyphenyl)boronic acid

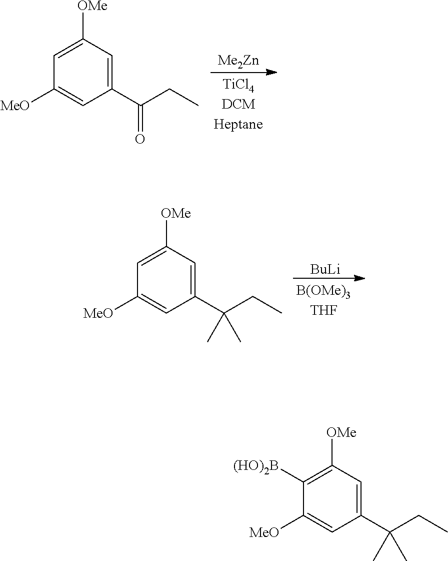

### Step 1: 1,3-Dimethoxy-5-(tert-pentyl)benzene

Dichloromethane (30 mL) was cooled in a dry-ice/acetonitrile bath under nitrogen until the internal temperature was below −40 °C. Titanium (IV) chloride solution (1 M in dichloromethane, 12.9 mL, 12.9 mmol) was added dropwise, followed by dimethylzinc solution (1 M in heptane, 12.9 mL, 12.9 mmol), keeping the temperature below −45 °C. A solution of 1-(3,5-dimethoxyphenyl) propan-1-one (1000 mg, 5.15 mmol) in dichloromethane (5 mL) was added and the reaction mixture was allowed to warm to room temperature overnight. The reaction was quenched by addition of water (10 ml), poured into water (100 ml) and extracted with dichloromethane (2×100 mL). The combined organic layers were dried (phase separating paper) and concentrated in vacuo. The residue was purified by column chromatography on silica gel, eluting with 0-50% diethyl ether in cyclohexane to give the title compound as a colourless oil (699 mg, 65%). ¹H NMR (400 MHz, CDCl₃) δ 6.49 (d, J=2.0 Hz, 2H), 6.30 (t, J=2.2 Hz, 1H), 3.80 (s, 6H), 1.61 (q, J=7.3 Hz, 2H), 1.25 (s, 6H), 0.69 (t, J=7.3 Hz, 3H).

### Step 2: (4-(tert-Pentyl)-2,6-dimethoxyphenyl)boronic acid

1-tert-pentyl-3,5-dimethoxy-benzene was borylated according to General Method A to give the title compound as a white solid. ¹H NMR (400 MHz, CDCl₃) δ 7.18 (s, 2H), 6.58 (s, 2H), 3.92 (s, 6H), 1.65 (q, J=7.4 Hz, 2H), 1.29 (s, 6H), 0.71 (t, J=7.5 Hz, 3H)

### Intermediate 4: (2,6-Bis(benzyloxy)-4-propylphenyl)boronic acid

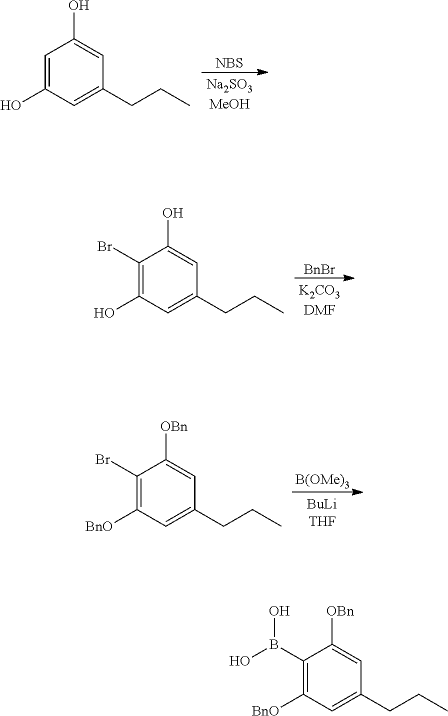

### Step 1: 2-Bromo-5-propylbenzene-1,3-diol

1,3-Dibenzyloxy 5-Propylbenzene-1,3-diol (22.05 g, 0.145 mol) in methyl alcohol (450 mL) was cooled in an ice/water bath and stirred. N-Bromosuccinimide (77.36 g, 0.435 mol) was added in portions over 1 hour maintaining an internal temperature of below 10 °C. On complete addition the reaction was stirred at room temperature for 20 h. The reaction was cooled in an ice-salt bath and a solution of sodium sulfite (40.2 g, 318 mmol) and NaOH (12.7 g, 317 mmol) in water (600 mL) was added in portions over 10 minutes. The reaction mixture was stirred for 2 hours then acidified with 1 M aqueous hydrochloric acid (220 mL) and extracted with ethyl acetate (2×600 mL). The combined organic layers were dried (magnesium sulfate) and the residue was purified by column chromatography on silica, eluting with 0-30% ethyl acetate in cyclohexane to give the title compound as a white solid (28.4 g, 84%). ¹H NMR (400 MHz, CDCl₃) δ 6.45 (s, 2H), 5.25 (s, 2H), 2.48 (t, J=7.6 Hz, 2H), 1.61-1.57 (m, 2H), 0.95-0.90 (t, J=7.2 Hz, 3H).

### Step 2: (((2-Bromo-5-propyl-1,3-phenylene)bis(oxy))bis(methylene))dibenzene

2-Bromo-5-propylbenzene-1,3-diol (28.40 g, 0.123 mol) in N,N-dimethylformamide (400 mL) was treated with potassium carbonate (84.93 g, 0.614 mol) followed by benzyl bromide (32 mL, 0.270 mol). The reaction mixture was stirred for 90 minutes, then concentrated in vacuo and the residue was partitioned between water (1 L) and diethyl ether (1 L). The layers were separated and the aqueous layer was extracted with diethyl ether (3×400 mL). The combined organic layers were washed with 4% LiCl (300 mL), dried (magnesium sulfate) and concentrated in vacuo. The residue triturated with n-pentane (500 mL) to give the title compound as a pink solid (46.4 g, 92%). ¹H NMR (400 MHz, CDCl₃) δ 7.49 (d, J=7.3 Hz, 4H), 7.41-7.31 (m, 6H), 6.45 (s, 2H), 5.15 (s, 4H), 2.50 (t, J=7.6 Hz, 2H), 1.63-1.53 (m, 2H), 0.88 (t, J=7.3 Hz, 3H).

### Step 3: (2,6-Bis(benzyloxy)-4-propylphenyl)boronic acid

(((2-Bromo-5-propyl-1,3-phenylene)bis(oxy))bis(methylene))dibenzene (20.0 g, 48.6 mmol) in tetrahydrofuran (750 mL) was cooled in a dry ice/acetone bath and treated with 2.5 M n-Butyllithium solution (29 mL, 72.9 mmol). After 5 minutes trimethyl borate (16 mL, 0.146 mol) was added and the reaction mixture was allowed to warm to room temperature overnight. The reaction mixture was quenched with saturated aqueous ammonium chloride (300 mL), poured into water (1 L) an extracted with ethyl acetate (3×500 mL). The combined organic layers were dried (magnesium sulfate) and concentrated in vacuo. The residue was purified by column chromatography silica gel, eluting with 5-30% ethyl acetate in cyclohexane to give the title compound as a white solid (11.36 g, 42%). ¹H NMR (400 MHz, CDCl₃) δ 7.42-7.36 (m, 10H), 7.14 (s, 2H), 6.54 (s, 2H), 5.13 (s, 4H), 2.57 (t, J=7.6 Hz, 2H), 1.69-1.59 (m, 2H), 0.93 (t, J=7.3 Hz, 3H).

### Intermediate 5: (4-(Cyclopent-1-en-1-yl)-2,6-dimethoxyphenyl)boronic acid

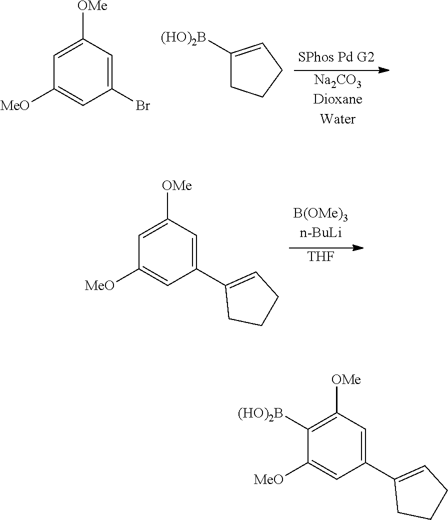 [SMILES: COc1cc(Br)cc(OC)c1 ; OB(O)C1=CCCC1 ; "Na2CO3DioxaneWater" ; COc1cc(OC)cc(C2=CCCC2)c1 ; COc1cc(C2=CCCC2)cc(OC)c1B(O)O ; "THF"]

### Step 1:1-(Cyclopent-1-en-1-yl)-3,5-dimethoxybenzene

A mixture of 1-bromo-3,5-dimethoxybenzene (1.50 g, 6.91 mmol), cyclopenten-1-ylboronic acid (928 mg, 8.29 mmol) and potassium carbonate (2.87 g, 20.7 mmol) in 1,4-dioxane (12 mL) and water (4 mL) was degassed with nitrogen and treated with SPhos Pd G2 (249 mg, 0.346 mmol). The reaction mixture was heated in a microwave reactor at 120 °C for 30 minutes then diluted with ethyl acetate (100 mL), washed with water (2×10⁰ mL), dried (phase separating paper) and concentrated in vacuo. The residue was purified by column chromatography on silica gel, eluting with 0-30% diethyl ether in cyclohexane to give 1-(cyclopenten-1-yl)-3,5-dimethoxy-benzene (1.23 g, 87%) as a white solid. ¹H NMR (400 MHz, CDCl₃) δ 6.60 (d, J=2.3 Hz, 2H), 6.35 (t, J=2.3 Hz, 1H), 6.19-6.15 (m, 1H), 3.80 (s, 6H), 2.72-2.65 (m, 2H), 2.55-2.48 (m, 2H), 2.05-1.97 (m, 2H).

### Step 2: (4-(Cyclopent-1-en-1-yl)-2,6-dimethoxyphenyl)boronic acid

1-(Cyclopenten-1-yl)-3,5-dimethoxy-benzene was borylated using General Method A to give the title compound as a white solid. ¹H NMR (400 MHz, DMSO) δ 7.80 (s, 2H), 6.63 (s, 2H), 6.32-6.28 (m, 1H), 3.73 (s, 6H), 2.72-2.66 (m, 2H), 2.51-2.46 (m, 2H, partly obscured by DMSO peak), 2.02-1.92 (m, 2H).

### Intermediate 6:8-Bromo-2,7-dimethylimidazo[1,2-a]pyridine

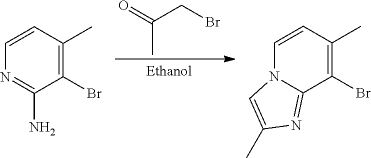

3-Bromo-4-methyl-pyridin-2-amine (400 mg, 2.14 mmol) and 1-bromopropan-2-one (439 mg, 3.21 mmol) were stirred in ethanol (8 mL) at 70 °C for 2 hours and cooled to room temperature. Saturated aqueous sodium hydrogen carbonate solution (20 mL), ethyl acetate (20 mL) and water (10 mL) were added. The aqueous phase was extracted with ethyl acetate (20 mL). The combined organic phases were filtered through a hydrophobic frit and concentrated in vacuo. The residue was purified by column chromatography on silica gel, eluting with ethyl acetate:ethanol (3:1) in cyclohexane to give the title compound (348 mg, 72%) as a pale pink solid. ¹H NMR (400 MHz, DMSO) δ 8.42 (d, J=6.8 Hz, 1H) 7.78-7.76 (m, 1H), 6.85 (d, J=6.8, 1H), 2.56 (s, 3H), 2.38-2.36 (m, 3H).

### General Method C

General Method C is illustrated using the synthesis of Intermediate 7.

### Intermediate 7:8-Bromo-6-chloro-7-methylimidazo[1,2-a]pyridine

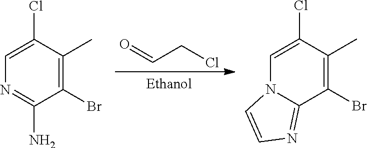

To a solution of 3-bromo-5-chloro-4-methyl-pyridin-2-amine (200 mg, 0.903 mmol) in ethanol (3.6 mL) was added chloroacetaldehyde solution (50 wt % in water) (0.17 mL, 1.35 mmol) and the reaction mixture was stirred at 70 °C for 1 day. The reaction mixture was cooled to room temperature and ethyl acetate (3 mL) was added followed by saturated aqueous sodium bicarbonate solution (3 mL) slowly and carefully and a colourless solid precipitated. Ethyl acetate (10 mL) and water (10 mL) were added and the organic phase was separated and the aqueous phase was extracted with ethyl acetate (10 mL). The combined organic phases were filtered through a hydrophobic frit and the solvent was concentrated in vacuo to give the title compound (224 mg, quantitative) as a beige solid. ¹H NMR (400 MHz, CDCl₃) δ 8.20 (s, 1H), 7.66 (d, J=1.0 Hz, 1H), 7.59 (d, J=1.3 Hz, 1H), 2.60 (s, 3H).

General Method C, using the appropriate aminopyridine, was used to generate the following intermediates.

### Intermediate 8:8-Bromo-7-methylimidazo[1,2-a]pyridine

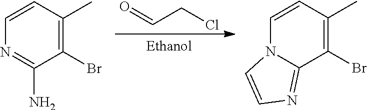

¹H NMR (400 MHz, CDCl₃) δ 7.99 (d, J=6.8 Hz, 1H), 7.63 (d, J=1.2 Hz, 1H), 7.59 (d, J=1.3 Hz, 1H), 6.67 (d, J=6.9 Hz, 1H), 2.49 (s, 3H).

### Intermediate 9:8-Chloro-7-(trifluoromethyl) imidazo[1,2-a]pyridine

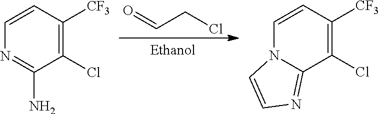

¹H NMR (400 MHz, CDCl₃) δ 8.17 (d, J=7.1 Hz, 1H), 7.84 (s, 1H), 7.75 (s, 1H), 7.07 (d, J=7.1 Hz, 1H).

### Intermediate 10:8-Bromo-6,7-dimethylimidazo[1,2-a]pyridine

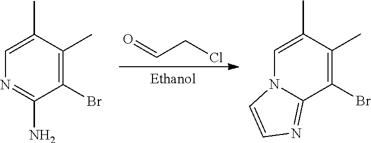

¹H NMR (400 MHz, CDCl₃) δ 7.86 (s, 1H), 7.59 (d, J=1.1 Hz, 1H), 7.52 (d, J=0.9 Hz, 1H), 2.46 (s, 3H), 2.30 (s, 3H).

### Intermediate 11:8-Bromo-7-fluoroimidazo[1,2-a]pyridine

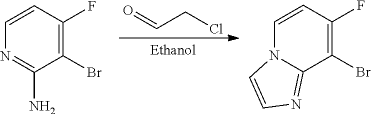

¹H NMR (400 MHz, CDCl₃) δ 8.07 (dd, J=5.0, 7.3 Hz, 1H), 7.68 (d, J=1.2 Hz, 1H), 7.64 (d, J=1.3 Hz, 1H), 6.76 (t, J=7.1 Hz, 1H).

### Intermediate 12:8-Bromo-6-fluoro-7-methylimidazo[1,2-a]pyridine

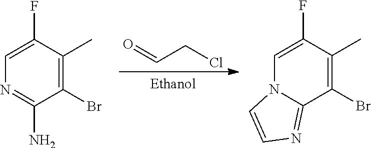

¹H NMR (400 MHz, CDCl₃) δ 8.04 (d, J=4.1 Hz, 1H), 7.67 (d, J=1.1 Hz, 1H), 7.62 (d, J=1.2 Hz, 1H), 2.47 (d, J=2.3 Hz, 3H).

### Intermediate 13:8-Bromo-7-methyl-2-(trifluoromethyl) imidazo[1,2-a]pyridine

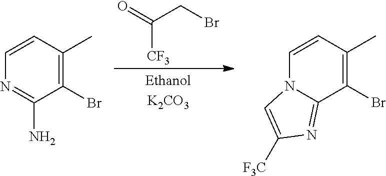

A suspension of 3-bromo-4-methyl-pyridin-2-amine (400 mg, 2.14 mmol), 3-bromo-1, 1,1-trifluoropropan-2-one (0.44 mL, 4.28 mmol) and potassium carbonate (443 mg, 3.21 mmol) in ethanol (20 mL) was heated at reflux overnight. The cooled reaction mixture was filtered and concentrated in vacuo. The residue was partitioned between a diluted aqueous sodium bicarbonate solution and dichloromethane. The organic phase was separated (hydrophobic frit) and concentrated in vacuo. The residue was purified by column chromatography on silica gel, eluting with 0-100% 3:1 ethanol/ethyl acetate in cyclohexane to give the title compound (275 mg, 46%) as a yellow solid. ¹H NMR (400 MHz, CDCl₃) δ 8.01 (d, J=6.8 Hz, 1H), 7.90 (s, 1H), 6.79 (1H, d, J=6.8 Hz, 1H), 2.52 (3H, s).

### Intermediate 14:8-Bromo-3,7-dimethylimidazo[1,2-a]pyridine

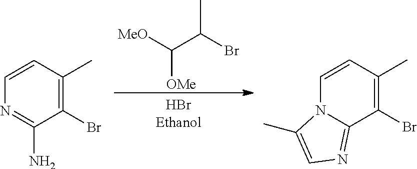

3-Bromo-4-methyl-pyridin-2-amine (500 mg, 2.67 mmol) in ethanol (5.0 mL) was treated with hydrobromic acid (48%, 0.45 mL, 3.98 mmol) and 2-bromo-1,1-dimethoxy-propane (1.1 mL, 7.98 mmol). The mixture was stirred at 80 °C for 3 days then treated with 0.5 M aqueous sodium carbonate solution (10 mL) and extracted with dichloromethane (2×15 mL). The combined organic layers were dried (magnesium sulfate) and concentrated in vacuo. The residue was purified by column chromatography on silica gel, eluting with 0-60% of a 3:1 mixture of ethyl acetate and ethanol in cyclohexane to give the title compound (222 mg, 35%) as a pale yellow solid. ¹H NMR (400 MHz, CDCl₃) δ 7.73 (d, J=7.1 Hz, 1H), 7.39 (s, 1H), 6.70 (d, J=8.1 Hz, 1H), 2.50 (s, 3H), 2.45 (s, 3H).

### Intermediate 15:8-Bromo-2-chloro-7-methylimidazo[1,2-a]pyridine

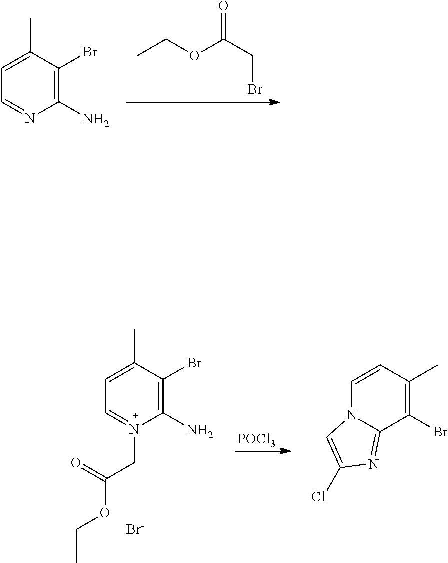

### Step 1:3-Bromo-1-(2-ethoxy-2-oxoethyl)-4-methylpyridin-1-ium bromide

3-Bromo-4-methyl-pyridin-2-amine (500 mg, 2.67 mmol) was suspended in ethyl bromoacetate (1.2 mL, 10.7 mmol) and stirred at room temperature for 1 day. The resulting solid was mobilised with diethyl ether (4 mL), collected by filtration, washed with further diethyl ether (2×5 mL) and dried under suction to give the title compound (869 mg, 92%) as a white solid. ¹H NMR (400 MHz, DMSO) δ 8.61 (s, 2H), 8.06 (d, J=6.9 Hz, 1H), 7.07 (d, J=6.9 Hz, 1H), 5.27 (s, 2H), 4.23 (q, J=7.1 Hz, 2H), 2.50 (s, 3H), 1.27 (t, J=7.1 Hz, 3H).

### Step 2:8-Bromo-2-chloro-7-methylimidazo[1,2-a]pyridine

3-Bromo-1-(2-ethoxy-2-oxoethyl)-4-methylpyridin-1-ium bromide (869 mg, 2.45 mmol) was suspended in phosphorus (V) oxychloride (2.3 mL, 24.5 mmol) and heated at 100 °C for 90 minutes, during which time the material dissolved. The reaction mixture was poured into ice-water (100 mL), neutralised with solid sodium carbonate and extracted with ethyl acetate (2×100 mL). The combined organic layers were dried (phase separating paper) and concentrated in vacuo to give the title compound (580 mg, 96%) as a pink solid. ¹H NMR (400 MHz, CDCl₃) δ 7.90 (d, J=6.7 Hz, 1H), 7.51 (s, 1H), 6.72 (d, J=6.8 Hz, 1H), 2.49 (s, 3H).

### Intermediate 16:8-Bromo-3-chloro-7-methylimidazo[1,2-a]pyridine

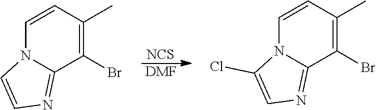

8-Bromo-7-methylimidazo[1,2-a]pyridine (250 mg, 1.18 mmol) in N,N-dimethylformamide (5 mL) was treated with N-chlorosuccinimide (174 mg, 1.30 mmol) and stirred overnight. The reaction mixture was diluted with ethyl acetate (50 mL), washed with water (3×50 mL), dried (phase separating paper) and concentrated in vacuo to the title compound as a light brown solid (243 mg, 84%) which was used without further purification in subsequent reactions. ¹H NMR (400 MHz, CDCl₃) δ 7.96 (d, J=6.8 Hz, 1H), 7.57 (s, 1H), 6.82 (d, J=6.8 Hz, 1H), 2.52 (s, 3H).

### Intermediate 17:8-Bromo-7-methyl-6-(trifluoromethyl) imidazo[1,2-a]pyridine

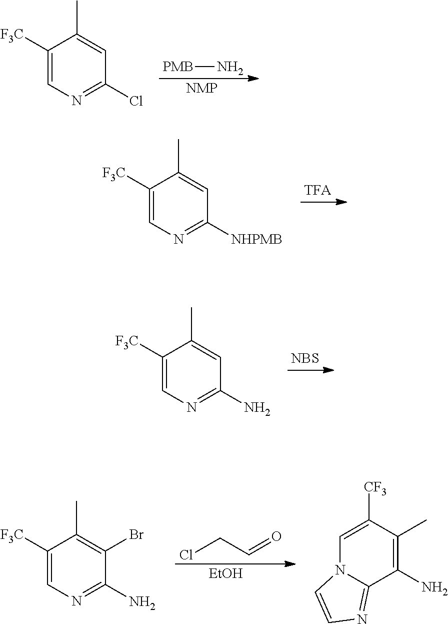

### Step 1: N-(4-Methoxybenzyl)-4-methyl-5-(trifluoromethyl)pyridin-2-amine

2-Chloro-4-methyl-5-(trifluoromethyl)pyridine (550 mg, 2.81 mmol) in 1-methyl-2-pyrrolidinone (10 mL) was treated with 4-methoxybenzylamine (1.5 mL, 11.2 mmol) and heated in a microwave reactor at 140 °C for 60 minutes. The reaction mixture was diluted with ethyl acetate (50 mL), washed with water (3×25 mL), dried (phase separating paper) and concentrated in vacuo. The residue was purified by column chromatography on silica gel, eluting with 0-50% ethyl acetate in cyclohexane to give the title compound as a white solid (555 mg, 67%). ¹H NMR (400 MHz, CDCl₃) δ 8.28 (s, 1H), 7.26 (d, J=8.5 Hz, 2H), 6.88 (d, J=8.5 Hz, 2H), 6.22 (s, 1H), 5.02 (s, 1H), 4.46 (d, J=4.3 Hz, 2H), 3.81 (s, 3H), 2.34 (s, 3H).

### Step 2:4-Methyl-5-(trifluoromethyl)pyridin-2-amine

N-[(4-Methoxyphenyl)methyl]-4-methyl-5-(trifluoromethyl)pyridin-2-amine (735 mg, 2.48 mmol) in trifluoroacetic acid (40 mL) was stirred overnight. The reaction mixture was concentrated in vacuo and partitioned between half-saturated aqueous sodium hydrogen carbonate solution (50 mL) and ethyl acetate (50 mL). The organic phase was washed with ethyl acetate (2×50 mL) and the combined organic layers were dried (phase separating paper) and concentrated in vacuo. The residue was purified by column chromatography on silica gel, eluting with 0-50% ethyl acetate in cyclohexane to give the title compound as a yellow solid (407 mg, 93%). ¹H NMR (400 MHz, CDCl₃) δ 8.24 (s, 1H), 6.35 (q, J=0.8 Hz, 1H), 4.65 (s, 2H), 2.37-2.35 (m, 3H).

### Step 3:3-Bromo-4-methyl-5-(trifluoromethyl)pyridin-2-amine

4-Methyl-5-(trifluoromethyl)pyridin-2-amine (387 mg, 2.20 mmol) in tetrahydrofuran (20 mL) was treated with N-bromosuccinimide (469 mg, 2.64 mmol) and stirred at room temperature for 90 minutes. The reaction mixture diluted with ethyl acetate (150 mL) and washed with half-saturated aqueous sodium thiosulfate (100 mL) and water (50 mL), dried (phase separating paper) and concentrated in vacuo to give the title compound as an off-white solid (619 mg, 91%). The crude material was used in the next step without further purification. ¹H NMR (400 MHz, CDCl₃) δ 8.21 (s, 1H), 5.33 (s, 2H), 2.48 (q, J=1.1 Hz, 3H). NMR contains 30 mol % succinimide; data recorded for major component only.

### Step 4:8-Bromo-7-methyl-6-(trifluoromethyl) imidazo[1,2-a]pyridine

3-Bromo-4-methyl-5-(trifluoromethyl)pyridin-2-amine was condensed with chloroacetaldehyde using a method similar to General Method C to give the title compound. ¹H NMR (400 MHz, CDCl₃) δ 8.48 (s, 1H), 7.74 (d, J=1.3 Hz, 1H), 7.70 (d, J=1.4 Hz, 1H), 2.62 (q, J=1.3 Hz, 3H).

### Intermediate 18:8-Chloro-7-cyclopropylimidazo[1,2-a]pyridine

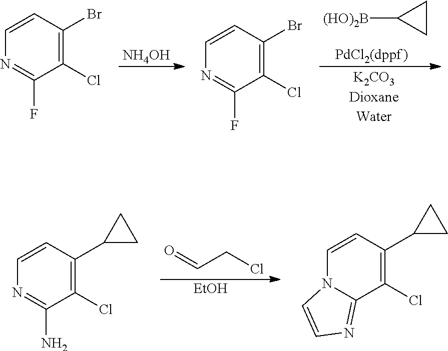

### Step 1:4-Bromo-3-chloropyridin-2-amine

4-Bromo-3-chloro-2-fluoropyridine (1.12 g, 5.32 mmol) in ammonium hydroxide solution (35%, 7 mL, 69.2 mmol) was heated in a reinforced 40 ml tube at 110 °C for 2 hours, then cooled. The reaction mixture was diluted with ethyl acetate (40 mL) and washed with brine (40 mL) was added. The aqueous layer was extracted with ethyl acetate (40 mL) and the combined organic layers were dried (phase separating paper) and concentrated in vacuo to give a colourless solid (710 mg). The mixture was redissolved in ammonium hydroxide solution, (35%, 7.0 mL, 69.2 mmol) and was stirred and heated in a reinforced 40 mL tube at 115 °C for 4 hours then cooled to room temperature. The reaction mixture was diluted with ethyl acetate (40 mL) and washed with brine (40 mL). The aqueous layer was extracted with ethyl acetate (40 mL) and the combined organic layers were dried (phase separating paper) and concentrated in vacuo to give the title compound as a colourless solid. (559 mg, 63% purity, remainder starting material) which was used in the next step without further purification. ¹H NMR (400 MHz, CDCl₃) δ 7.77 (8, J=5.3 Hz, 1H), 6.92 (d, J=5.4 Hz, 1H), 5.00 (s, 2H). Data reported for major component only.

### Step 2:3-Chloro-4-cyclopropylpyridin-2-amine

4-Bromo-3-chloropyridin-2-amine (63% purity, 469 mg, 2.26 mmol), cyclopropyl-boronic acid (194 mg, 2.26 mmol) and potassium carbonate (937 mg, 6.78 mmol) in 1,4-dioxane (13 mL) and water (4.5 mL) were treated with [1,1′-bis(diphenylphosphino)-ferrocene]dichloropalladium(II) (83 mg, 0.113 mmol) and heated at 100 °C overnight. The reaction mixture was combined with that from a test reaction which had been run under the same conditions using 54 mg of starting material, filtered through celite and washed through with ethyl acetate (40 mL). The filtrate was washed with saturated aqueous sodium bicarbonate solution (40 mL), the layers were separated and the aqueous layer was extracted with ethyl acetate (40 mL). The combined organic layers were dried (phase separating paper) and concentrated in vacuo. The residue was purified by column chromatography on silica gel, eluting with 0-5% methanol in dichloromethane to give the title compound (156 mg, c 70% purity) as a yellow solid.

### Step 3:8-Chloro-7-cyclopropylimidazo[1,2-a]pyridine

3-Chloro-4-cyclopropylpyridin-2-amine (70% purity, 156 mg, 0.925 mmol) and chloroacetaldehyde solution (50 wt % in water, 0.18 mL, 1.39 mmol) in ethanol (3 mL) were heated in a sealed tube at 70 °C overnight. The reaction mixture was partitioned between saturated aqueous sodium bicarbonate solution (10 mL) and ethyl acetate (10 mL) and the layers were separated. The aqueous layer was extracted with ethyl acetate (10 mL) and the combined organic layers were dried (phase separating paper) and concentrated in vacuo to give a brown oil. The residue was purified by column chromatography on silica gel, eluting with 20-100% ethyl acetate in cyclohexane to give the title compound (108 mg, c 80% purity) as a brown oil, which was used without further purification in subsequent reactions. ¹H NMR (400 MHz, CDCl₃) δ 7.94 (d, J=7.1 Hz, 1H), 7.62 (d, J=1.1 Hz, 1H), 7.53 (d, J=1.0 Hz, 1H), 6.26 (d, J=7.1 Hz, 1H), 2.45-2.37 (m, 1H), 1.16-1.11 (m, 2H), 0.80-0.75 (m, 2H). Data reported for major component only.

### Synthesis of Final Compounds

### General Method D

### Compound 1:2-(imidazo[1,2-a]pyridin-8-yl)-5-propylbenzene-1,3-diol

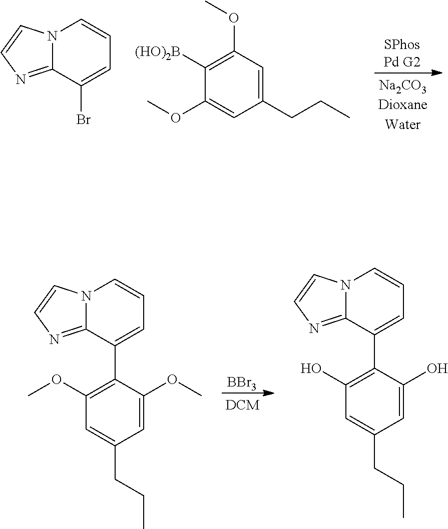

### Step 1:8-(2,6-Dimethoxy-4-propylphenyl) imidazo[1,2-a]pyridine

(2,6-Dimethoxy-4-propyl-phenyl)boronic acid (Intermediate 1; 200 mg, 0.893 mmol), 8-bromoimidazo[1,2-a]pyridine (Combi-Blocks Inc; 229 mg, 1.16 mmol) and potassium carbonate (370 mg, 2.68 mmol) in 1,4-dioxane (3 mL) and water (1 mL) were treated with SPhos Pd G2 (64 mg, 0.0893 mmol) and heated in a microwave reactor at 120 °C for 1 hour. The reaction mixture was diluted with ethyl acetate (20 mL) and saturated aqueous sodium bicarbonate solution (20 mL). The layers were separated and the aqueous layer was extracted with ethyl acetate (20 mL). The combined organic layers were dried (phase separating paper) and concentrated in vacuo. The residue was purified by column chromatography, eluting with 40-100% ethyl acetate in cyclohexane to give the title compound as a white solid (222 mg, 84%). ¹H NMR (400 MHz, CDCl₃) δ 8.10 (dd, J=1.2, 6.8 Hz, 1H), 7.59 (d, J=1.2 Hz, 1H), 7.57 (d, J=1.2 Hz, 1H), 7.04 (dd, J=1.2, 6.8 Hz, 1H), 6.83 (t, J=6.8 Hz, 1H), 6.51 (s, 2H), 3.69 (s, 6H), 2.62 (t, J=7.8 Hz, 2H), 1.75-1.66 (m, 2H), 1.02 (t, J=7.3 Hz, 3H).

### Step 2:2-(Imidazo[1,2-a]pyridin-8-yl)-5-propylbenzene-1,3-diol

8-(2,6-Dimethoxy-4-propyl-phenyl) imidazo[1,2-a]pyridine (222 mg, 0.749 mmol) in dichloromethane (5 mL) was cooled in an ice/water bath and boron tribromide (2.6 mL, 1M solution in dichloromethane, 2.6 mmol) was added and the reaction mixture allowed to warm to room temperature over 2 hours then cooled to 0 °C and quenched with methanol (4 mL), followed by saturated aqueous sodium bicarbonate solution (20 mL). Ethyl acetate (20 mL) was added and the layers were separated. The aqueous layer was extracted with ethyl acetate (20 mL) and the combined organic layers were dried (phase separating paper) and concentrated in vacuo. The residue was dissolved in methanol (4 mL) and ammonia in methanol (7N, 4 mL), stirred at room temperature for 30 minutes and concentrated in vacuo. The residue was purified by preparative HPLC to give the title compound as a purple solid (79 mg, 39%). ¹H NMR (400 MHz, DMSO) δ 9.24 (s, 2H), 8.54 (dd, J=1.3, 6.8 Hz, 1H), 7.98 (d, J=1.1 Hz, 1H), 7.52 (d, J=1.0 Hz, 1H), 7.24 (dd, J=1.3, 7.1 Hz, 1H), 6.96 (t, J=6.9 Hz, 1H), 6.29 (s, 2H), 2.44 (t, J=7.6 Hz, 2H), 1.65-1.55 (m, 2H), 0.95 (t, J=7.3 Hz, 3H). MS (ESI): m/z 269.2 (M+1). HPLC purity: 98.4%.

The following final compounds were prepared in an analogous manner to Compound 1, reacting the appropriate aryl halide and boronic acid via Suzuki cross-coupling chemistry.

### Compound 2:2-(7-Methylimidazo[1,2-a]pyridin-8-yl)-5-propylbenzene-1,3-diol

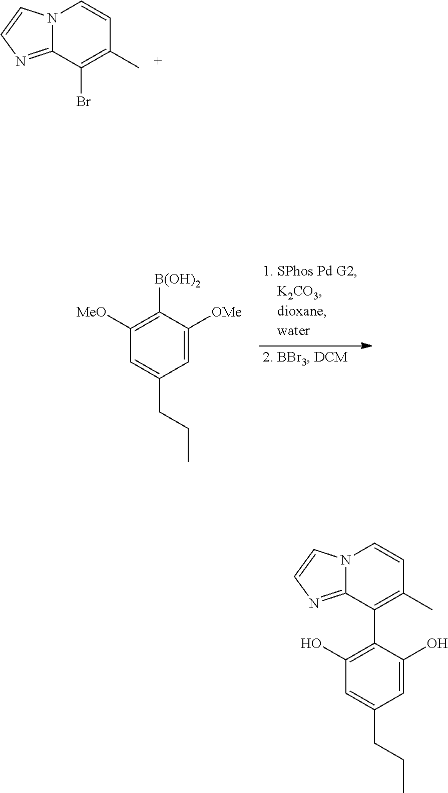 [SMILES: Cc1ccn2ccnc2c1Br ; CCCc1cc(OC)c(B(O)O)c(OC)c1 ; CCCc1cc(O)c(-c2c(C)ccn3ccnc23)c(O)c1]

Compound 2 was prepared from Intermediates 1 and 8.

¹H NMR (400 MHz, DMSO) δ 8.78 (s, 2H), 8.37 (d, J=6.8 Hz, 1H), 7.81 (d, J=1.0 Hz, 1H), 7.32 (d, J=0.9 Hz, 1H), 6.78 (d, J=6.9 Hz, 1H), 6.24 (s, 2H), 2.45 (t, J=7.7 Hz, 2H), 2.05 (s, 3H), 1.65-1.56 (m, 2H), 0.97 (t, J=7.3 Hz, 3H). MS (ESI): m/z 283.5 (M+1). HPLC purity: 99.5%.

### Compound 3: 2-(2,7-Dimethylimidazo[1,2-a]pyridin-8-yl)-5-propylbenzene-1,3-diol

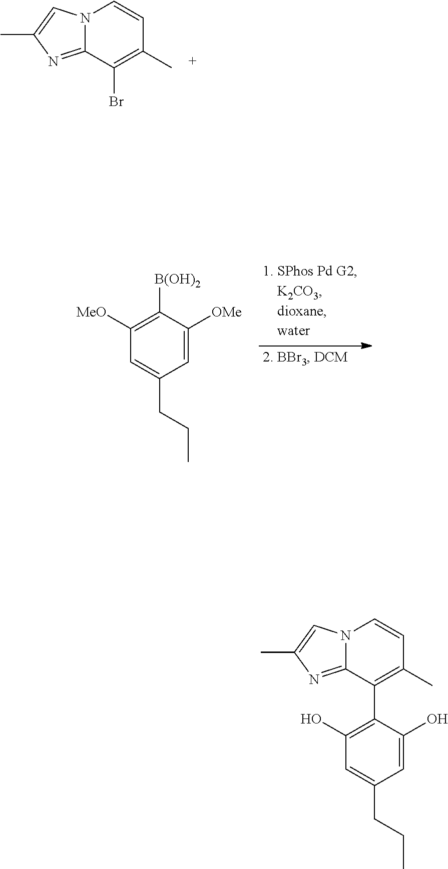

Compound 3 was prepared from Intermediates 1 and 6.

¹H NMR (400 MHz, DMSO) δ 8.28 (d, J=6.9 Hz, 1H), 8.17 (s, 1H), 7.57 (d, J=0.9 Hz, 1H), 6.74 (d, J=6.9 Hz, 1H), 6.25 (s, 2H), 2.45 (t, J=7.6 Hz, 2H), 2.23 (s, 3H), 2.04 (s, 3H), 1.65-1.58 (m, 2H), 0.97 (t, J=7.3 Hz, 3H), (2×OH not observed). MS (ESI): m/z 297.5 (M+1). HPLC purity: 99.5%. Isolated as a 1.0 equivalent formate salt.

### Compound 4: 2-(6-Chloro-7-methylimidazo[1,2-a]pyridin-8-yl)-5-propylbenzene-1,3-diol

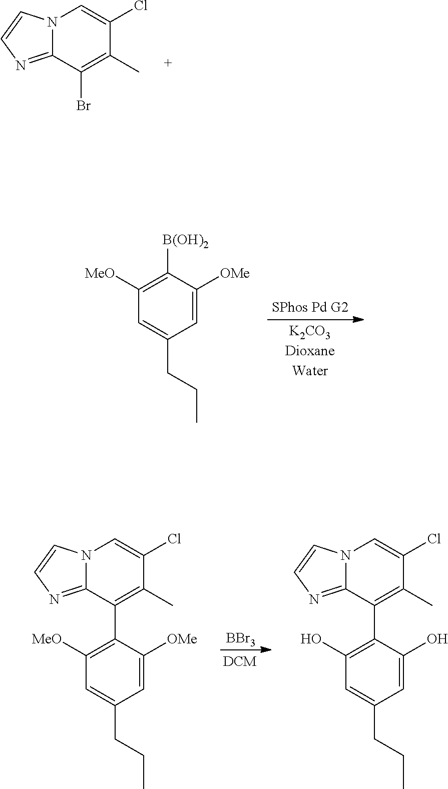

### 100-Milligram Scale Synthesis of Compound 4

### Step 1:6-Chloro-8-(2,6-dimethoxy-4-propylphenyl)-7-methylimidazo[1,2-a]pyridine

(2,6-Dimethoxy-4-propyl-phenyl)boronic acid (Intermediate 1; 100 mg, 0.446 mmol), 8-bromo-6-chloro-7-methylimidazo[1,2-a]pyridine (Intermediate 7; 142 mg, 0.580 mmol), and potassium carbonate (185 mg, 1.34 mmol) in dioxane (1.5 mL) and water (0.5 mL) were degassed with nitrogen and treated with SPhos Pd G2 (32 mg, 0.045 mmol). The reaction mixture was heated in a microwave reactor at 120 °C for 30 minutes, then combined with that of a test reaction which had been run on 50 mg scale under identical conditions, and filtered through celite. The celite pad was washed with ethyl acetate (20 mL) followed by saturated sodium bicarbonate solution (20 mL). The layers of the filtrate were separated and the aqueous layer was extracted with ethyl acetate (2×20 mL). The combined organic layers were washed with brine (20 mL), dried (magnesium sulfate) and concentrated in vacuo. The residue was purified by column chromatography to give the title compound as a pale orange solid (138 mg, 60%). ¹H NMR (400 MHz, CDCl₃) δ 8.19 (s, 1H), 7.53 (d, J=1.1 Hz, 1H), 7.47 (d, J=1.3 Hz, 1H), 6.51 (s, 2H), 3.68 (s, 6H), 2.63 (t, J=7.8 Hz, 2H), 2.14 (s, 3H), 1.76-1.67 (m, 2H), 1.02 (t, J=7.3 Hz, 3H).

### Step 2:2-(6-Chloro-7-methylimidazo[1,2-a]pyridin-8-yl)-5-propylbenzene-1,3-diol

6-Chloro-8-(2,6-dimethoxy-4-propylphenyl)-7-methylimidazo[1,2-a]pyridine (138 mg, 0.400 mmol) in dichloromethane (4 mL) was treated with 1 M boron tribromide in dichloromethane (2.0 mL, 2.00 mmol) and stirred overnight. The reaction mixture was cooled to −78 °C and quenched with methanol (4 mL). The mixture was warmed to room temperature and saturated sodium bicarbonate (4 mL) and 7N ammonia in methanol (3 mL) were added. The organic layer was separated and the aqueous layer was extracted with dichloromethane (2×8 mL). The combined organic layers were dried (hydrophobic frit) and the solvent was removed in vacuo. The residue was purified by reverse phase preparative HPLC to give the title compound as an off white solid (43 mg, 34%). ¹H NMR (400 MHz, DMSO) δ 8.91 (s, 2H), 8.81 (s, 1H), 7.85-7.83 (m, 1H), 7.41 (s, 1H), 6.26 (s, 2H), 2.45 (t, J=7.6 Hz, 2H), 2.09 (s, 3H), 1.67-1.56 (m, 2H), 0.97 (t, J=7.3 Hz, 3H). MS (ESI): m/z 317.2 (M+1). HPLC purity: 98.6%.

### 1-Gram Scale Synthesis of Compound 4

### Step 1:6-Chloro-8-(2,6-dimethoxy-4-propylphenyl)-7-methylimidazo[1,2-a]pyridine

8-Bromo-6-chloro-7-methyl-imidazo[1,2-a]pyridine (Intermediate 7; 1000 mg, 4.07 mmol), (2,6-dimethoxy-4-propyl-phenyl)boronic acid (Intermediate 1; 1369 mg, 6.11 mmol) and potassium carbonate (1689 mg, 12.2 mmol) in 1,4-dioxane (12 mL) and water (4 mL) were degassed with nitrogen, treated with SPhos Pd G2 (294 mg, 0.407 mmol) and heated in a microwave reactor at 150 °C for 45 minutes. Four further reactions were carried out on the same scale and under the same conditions. The five reaction mixtures were combined, diluted with ethyl acetate (350 ml), washed with water (2×100 mL), dried (hydrophobic filter paper) and concentrated in vacuo. The residue was purified by column chromatography on silica gel, eluting with 30-60% ethyl acetate in cyclohexane, to give the title compound as an off-white solid (3710 mg, 53%). ¹H NMR (400 MHz, CDCl₃) δ 8.19 (s, 1H), 7.53 (d, J=1.3 Hz, 1H), 7.47 (d, J=1.1 Hz, 1H), 6.52 (s, 2H), 3.68 (s, 6H), 2.63 (t, J=7.7 Hz, 2H), 2.15 (s, 3H), 1.77-1.67 (m, 2H), 1.02 (t, J=7.3 Hz, 3H).

### Step 2: 2-(6-Chloro-7-methylimidazo[1,2-a]pyridin-8-yl)-5-propylbenzene-1,3-diol

6-Chloro-8-(2,6-dimethoxy-4-propyl-phenyl)-7-methyl-imidazo[1,2-a]pyridine (3250 mg, 9.42 mmol) in dichloromethane (60 mL) was cooled in an ice/water bath and treated with 1 M Boron tribromide in dichloromethane (28 mL, 28.3 mmol). The reaction mixture was allowed to warm to room temperature overnight then was cooled in a dry-ice/acetone bath, quenched with methanol (10 mL), warmed to room temperature and concentrated in vacuo. The residue was coevaporated with methanol (3×50 mL) followed by 7N ammonia in methanol (100 mL). The residue was partitioned between ethyl acetate (150 mL) and water (100 mL). The layers were separated, the aqueous layer was extracted with ethyl acetate (100 mL) and the combined organic layers were dried (hydrophobic filter paper) and concentrated in vacuo to give a light brown solid. This was slurried overnight at room temperature in degassed water (40 mL) and ethyl acetate (40 mL). The solid was collected by filtration, dried under vacuum and slurried in acetonitrile followed by water to remove traced of organic solvents and dried under vacuum to give the title compound as an off-white solid (2097 mg, 70%). ¹H NMR (400 MHz, DMSO) δ 8.89 (s, 2H), 8.79 (s, 1H), 7.82 (d, J=0.9 Hz, 1H), 7.39 (d, J=0.9 Hz, 1H), 6.24 (s, 2H), 2.44 (t, J=7.5 Hz, 2H), 2.08 (s, 3H), 1.66-1.55 (m, 2H), 0.95 (t, J=7.3 Hz, 3H). MS (ESI): m/z 317.4 (M+1). HPLC purity: 98.6%.

### Compound 5: 2-(7-trifluoromethylimidazo[1,2-a]pyridin-8-yl)-5-propylbenzene-1,3-diol

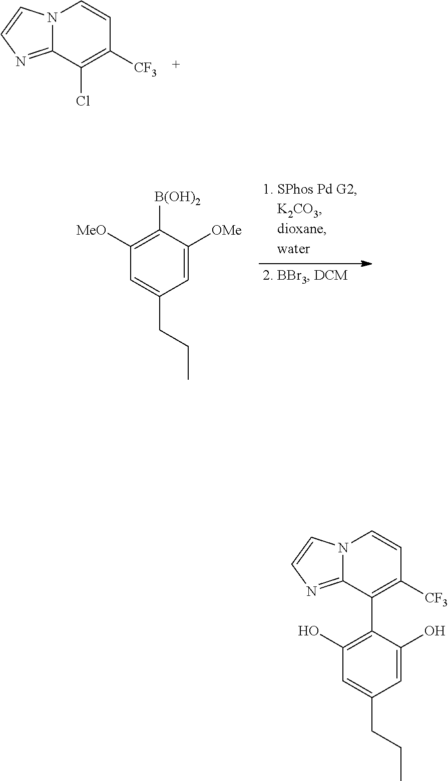 [SMILES: FC(F)(F)c1ccn2ccnc2c1Cl ; CCCc1cc(OC)c(B(O)O)c(OC)c1 ; CCCc1cc(O)c(-c2c(C(F)(F)F)ccn3ccnc23)c(O)c1]

Compound 5 was prepared from Intermediates 1 and 9.

¹H NMR (400 MHz, DMSO) δ 8.95 (s, 2H), 8.68 (d, J=6.9 Hz, 1H), 8.16 (s, 0.4H), 8.10 (d, J=1.0 Hz, 1H), 7.59 (d, J=1.1 Hz, 1H), 7.14 (d, J=7.3 Hz, 1H), 6.21 (s, 2H), 2.45 (t, J=7.7 Hz, 2H), 1.67-1.56 (m, 2H), 0.97 (t, J=7.4 Hz, 3H). MS (ESI): m/z 337.6 (M+1). HPLC purity: 99.8%. Isolated as a 0.4 equivalent formate salt.

### Compound 6: 2-(7-Chloroimidazo[1,2-a]pyridin-8-yl)-5-propylbenzene-1,3-diol

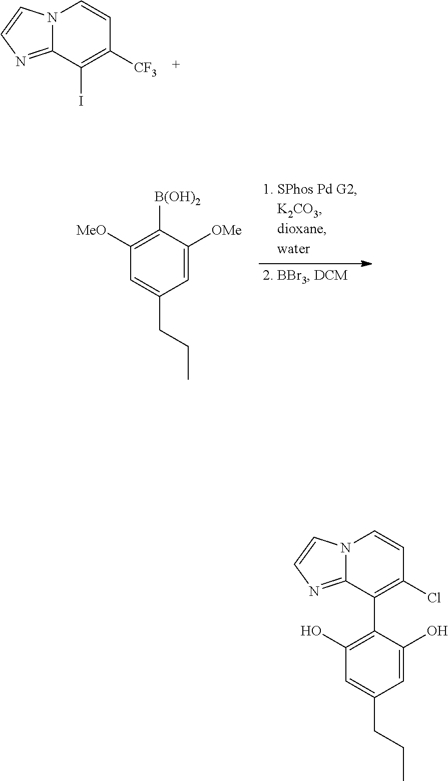 [SMILES: FC(F)(F)c1ccn2ccnc2c1I ; CCCc1cc(OC)c(B(O)O)c(OC)c1 ; CCCc1cc(O)c(-c2c(Cl)ccn3ccnc23)c(O)c1]

Compound 6 was prepared from Intermediate 1 and 7-chloro-8-iodoimidazo[1,2-a]pyridine which was sourced from Key Organics Ltd.

¹H NMR (400 MHz, DMSO) δ 12.79 (s, 0.5H), 8.96 (s, 2H), 8.51 (d, J=7.3 Hz, 1H), 8.15 (s, 0.5H), 7.94 (d, J=1.2 Hz, 1H), 7.43 (d, J=1.2 Hz, 1H), 7.01 (d, J=7.1 Hz, 1H), 6.24 (s, 2H), 2.45 (t, J=7.7 Hz, 2H), 1.67-1.56 (m, 2H), 0.97 (t, J=7.3 Hz, 3H). MS (ESI): m/z 303.3 (M+1). HPLC purity: 98.9%. Isolated as a 0.5 equivalent formate salt.

### Compound 7: 2-(6,7-Dimethylimidazo[1,2-a]pyridin-8-yl)-5-propylbenzene-1,3-diol

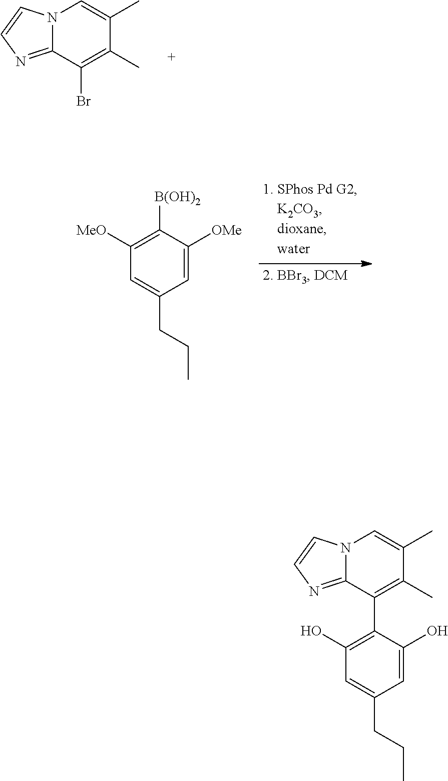 [SMILES: Cc1cn2ccnc2c(Br)c1C ; CCCc1cc(OC)c(B(O)O)c(OC)c1 ; CCCc1cc(O)c(-c2c(C)c(C)cn3ccnc23)c(O)c1]

Compound 7 was prepared from Intermediates 1 and 10.

¹H NMR (400 MHz, DMSO) δ 8.79 (s, 2H), 8.29 (s, 1H), 7.76 (s, 1H), 7.32 (s, 1H), 6.26 (s, 2H), 2.45 (t, J=7.6 Hz, 2H), 2.25 (s, 3H), 1.98 (s, 3H), 1.67-1.56 (m, 2H), 0.97 (t, J=7.4 Hz, 3H). MS (ESI): m/z 297.4 (M+1). HPLC purity: 99.5%.

### Compound 8: 2-(7-Fluoroimidazo[1,2-a]pyridin-8-yl)-5-propylbenzene-1,3-diol

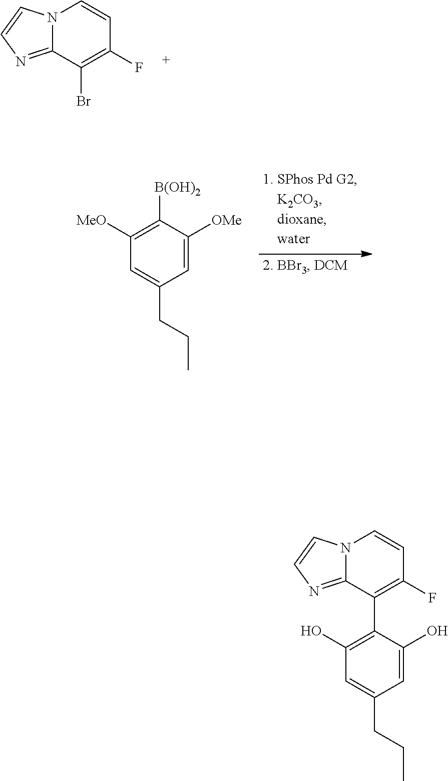 [SMILES: Fc1ccn2ccnc2c1Br ; CCCc1cc(OC)c(B(O)O)c(OC)c1 ; CCCc1cc(O)c(-c2c(F)ccn3ccnc23)c(O)c1]

Compound 8 was prepared from Intermediates 1 and 11.

¹H NMR (400 MHz, DMSO) δ 9.07 (s, 2H), 8.15 (s, 1H), 8.57 (dd, J=5.7, 7.4 Hz, 1H), 7.91 (d, J=1.3 Hz, 1H), 7.42 (d, J=1.3 Hz, 1H), 6.97 (t, J=7.7 Hz, 1H), 6.26 (s, 2H), 2.45 (t, J=7.5 Hz, 2H), 1.66-1.55 (m, 2H), 0.96 (t, J=7.3 Hz, 3H). MS (ESI): m/z 287.5 (M+1). HPLC purity: 99.5%. Isolated as a 1.0 equivalent formate salt.

### Compound 9: 2-(3,7-Dimethylimidazo[1,2-a]pyridin-8-yl)-5-propylbenzene-1,3-diol

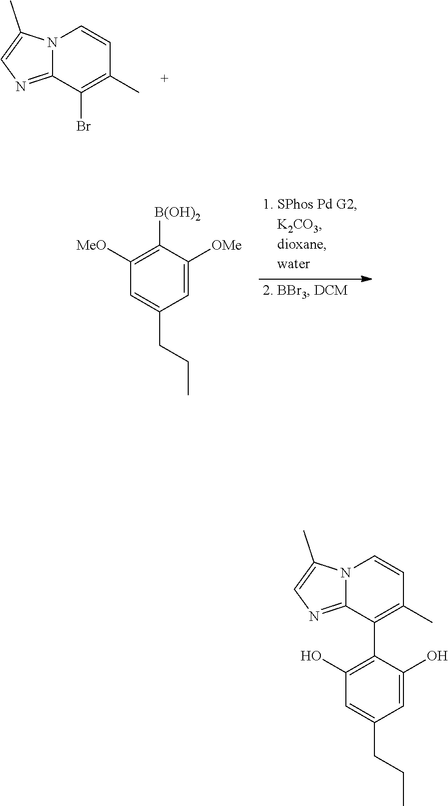 [SMILES: Cc1ccn2c(C)cnc2c1Br ; CCCc1cc(OC)c(B(O)O)c(OC)c1 ; CCCc1cc(O)c(-c2c(C)ccn3c(C)cnc23)c(O)c1]

Compound 9 was prepared from Intermediates 1 and 14.

¹H NMR (400 MHz, DMSO) δ 8.17 (s, 1H), 8.10 (d, J=6.6 Hz, 1H), 7.14 (d, J=0.8 Hz, 1H), 6.86 (d, J=7.0 Hz, 1H), 6.24 (s, 2H), 2.45 (m, 5H), 2.08 (s, 3H), 1.66-1.56 (m, 2H), 0.97 (t, J=7.4 Hz, 3H). Exchangeable phenolic protons not observed. MS (ESI): m/z 297.2 (M+1). HPLC purity: 99.5%. Isolated as a 1.0 equivalent formate salt.

### Compound 10: 2-(3-Chloro-7-methylimidazo[1,2-a]pyridin-8-yl)-5-propylbenzene-1,3-diol

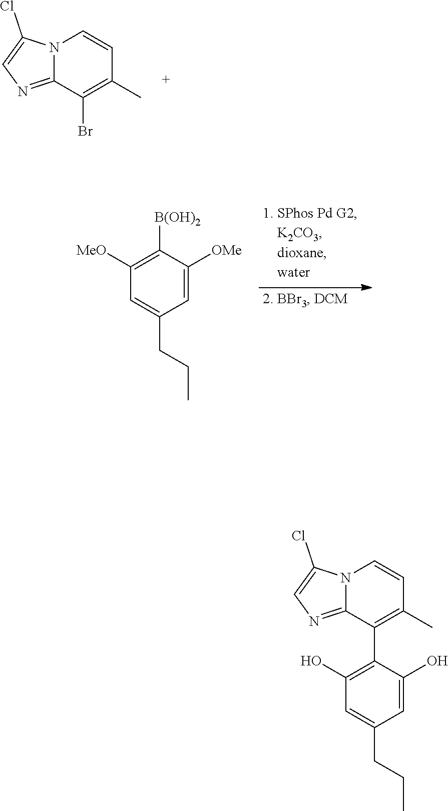 [SMILES: Cc1ccn2c(Cl)cnc2c1Br ; CCCc1cc(OC)c(B(O)O)c(OC)c1 ; CCCc1cc(O)c(-c2c(C)ccn3c(Cl)cnc23)c(O)c1]

Compound 10 was prepared from Intermediates 1 and 16.

¹H NMR (400 MHz, DMSO) δ 8.84 (s, 2H), 8.20 (d, J=7.3 Hz, 1H), 7.45 (s, 1H), 7.02 (d, J=6.9 Hz, 1H), 6.24 (s, 2H), 2.45 (t, J=7.7 Hz, 2H), 2.10 (s, 3H), 1.66-1.56 (m, 2H), 0.97 (t, J=7.3 Hz, 3H). MS (ESI): m/z 317.5 (M+1). HPLC purity: 99.2%.

### Compound 11: 2-(6-Fluoro-7-methylimidazo[1,2-a]pyridin-8-yl)-5-propylbenzene-1,3-diol

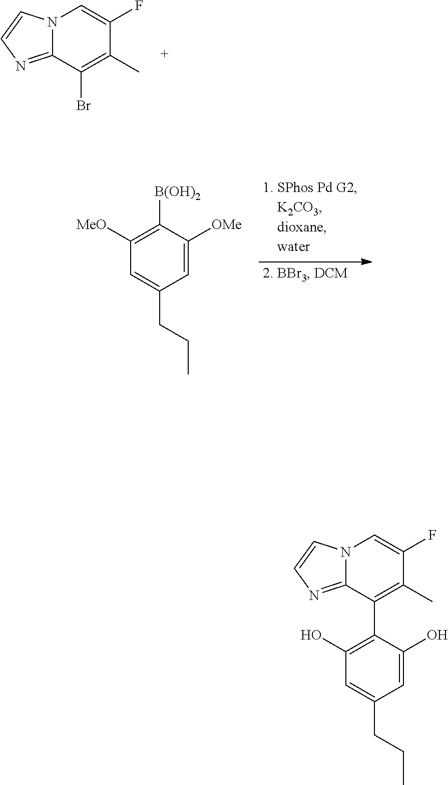 [SMILES: Cc1c(F)cn2ccnc2c1Br ; CCCc1cc(OC)c(B(O)O)c(OC)c1 ; CCCc1cc(O)c(-c2c(C)c(F)cn3ccnc23)c(O)c1]

Compound 11 was prepared from Intermediates 1 and 12.

¹H NMR (400 MHz, DMSO) δ 8.92 (s, 2H), 8.70-8.67 (m, 1H), 8.15 (s, 1H), 7.85 (s, 1H), 7.41 (s, 1H), 6.26 (s, 2H), 2.45 (t, J=7.6 Hz, 2H), 2.02-2.00 (m, 3H), 1.67-1.57 (m, 2H), 0.97 (t, J=7.3 Hz, 3H). MS (ESI): m/z 301.3 (M+1). HPLC purity: 95.8%. Isolated as a 1.0 equivalent formate salt.

### Compound 12: 2-(2-Chloro-7-methylimidazo[1,2-a]pyridin-8-yl)-5-propylbenzene-1,3-diol

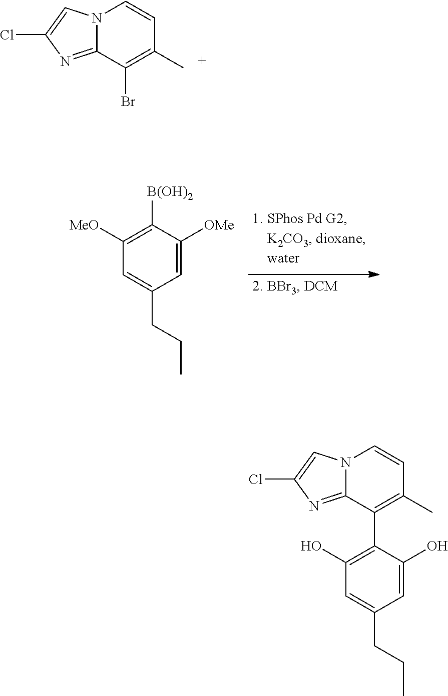 [SMILES: Cc1ccn2cc(Cl)nc2c1Br ; CCCc1cc(OC)c(B(O)O)c(OC)c1 ; CCCc1cc(O)c(-c2c(C)ccn3cc(Cl)nc23)c(O)c1]

Compound 12 was prepared from Intermediates 1 and 15.

¹H NMR (400 MHz, DMSO) δ 8.88 (s, 2H), 8.32 (d, J=6.9 Hz, 1H), 7.91 (s, 1H), 6.89 (d, J=6.9 Hz, 1H), 6.25 (s, 2H), 2.45 (t, J=7.4 Hz, 2H), 2.06 (s, 3H), 1.67-1.57 (m, 2H), 0.97 (t, J=7.7 Hz, 3H). MS (ESI): m/z 317.1 (M+1). HPLC purity: 98.6%.

### Compound 13: 2-(7-Methylimidazo[1,2-a]pyridin-8-yl)-5-pentylbenzene-1,3-diol

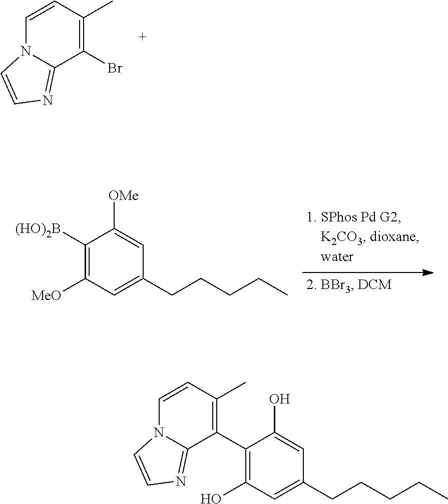 [SMILES: Cc1ccn2ccnc2c1Br ; CCCCCc1cc(OC)c(B(O)O)c(OC)c1 ; CCCCCc1cc(O)c(-c2c(C)ccn3ccnc23)c(O)c1]

Compound 13 was prepared from Intermediates 2 and 8.

¹H NMR (400 MHz, DMSO) δ 10.30 (br s, 2H), 8.36 (d, J=6.8 Hz, 1H), 8.15 (s, 1H), 7.80 (d, J=1.0 Hz, 1H), 7.32 (d, J=1.0 Hz, 1H), 6.78 (d, J=6.8 Hz, 1H), 6.23 (s, 2H), 2.44 (t, J=7.6 Hz, 2H), 2.04 (s, 3H), 1.62-1.53 (m, 2H), 1.37-1.30 (m, 4H), 0.90 (t, J=6.9 Hz, 3H). MS (ESI): m/z 311.5 (M+1). HPLC purity: 98.5%. Isolated as a 1 equivalent formate salt.

### Compound 14: 2-(7-Methylimidazo[1,2-a]pyridin-8-yl)benzene-1,3-diol

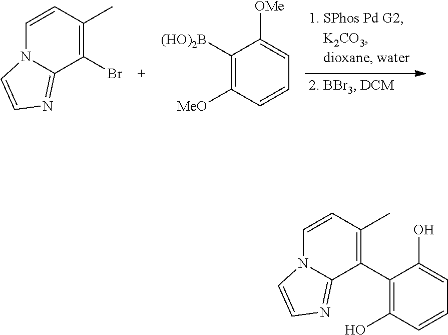 [SMILES: Cc1ccn2ccnc2c1Br ; COc1cccc(OC)c1B(O)O ; Cc1ccn2ccnc2c1-c1c(O)cccc1O]

Compound 14 was prepared from Intermediate 8 and 2,6-dimethoxybenzene boronic acid, which was sourced from Fluorochem Ltd.

¹H NMR (400 MHz, DMSO) δ 9.00 (s, 1H), 8.39 (d, J=7.0 Hz, 1H), 8.16 (s, 1H), 7.83 (d, J=1.1 Hz, 1H), 7.35 (d, J=1.1 Hz, 1H), 7.00 (t, J=8.1 Hz, 1H), 6.81 (d, J=7.0 Hz, 1H), 6.41 (d, J=8.1 Hz, 2H), 2.06 (s, 3H). MS (ESI): m/z 241.4 (M+1). HPLC purity: 99.4%. Isolated as a 1.0 equivalent formate salt.

### Compound 15: 2-(2-Trifluoromethyl-7-methylimidazo[1,2-a]pyridin-8-yl)-5-propylbenzene-1,3-diol

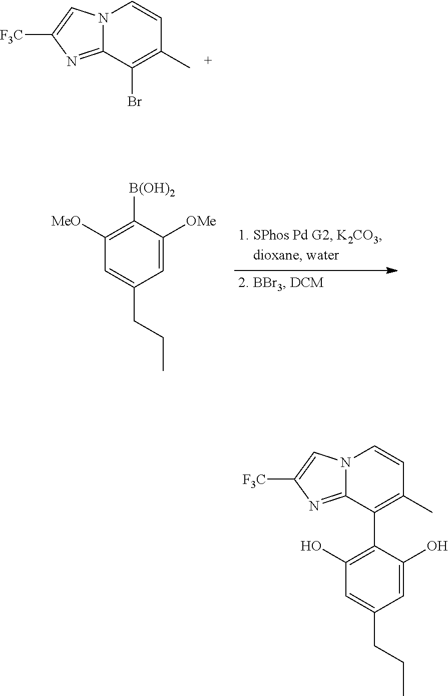 [SMILES: Cc1ccn2cc(C(F)(F)F)nc2c1Br ; CCCc1cc(OC)c(B(O)O)c(OC)c1 ; CCCc1cc(O)c(-c2c(C)ccn3cc(C(F)(F)F)nc23)c(O)c1]

Compound 15 was prepared from Intermediates 1 and 13.

¹H NMR (400 MHz, DMSO) δ 8.89 (s, 2H), 8.42 (d, J=6.9 Hz, 1H), 8.39 (s, 1H), 6.96 (d, J=6.9 Hz, 1H), 6.25 (s, 2H), 2.44 (t, J=7.6 Hz, 2H), 2.06 (s, 3H), 1.56-1.63 (m, 2H), 0.96 (t, J=7.3 Hz, 3H). MS (ESI): m/z 351.4 (M+1). HPLC purity: 98.7%.

### Compound 16: 2-(6-Trifluoromethyl-7-methylimidazo[1,2-a]pyridin-8-yl)-5-propylbenzene-1,3-diol

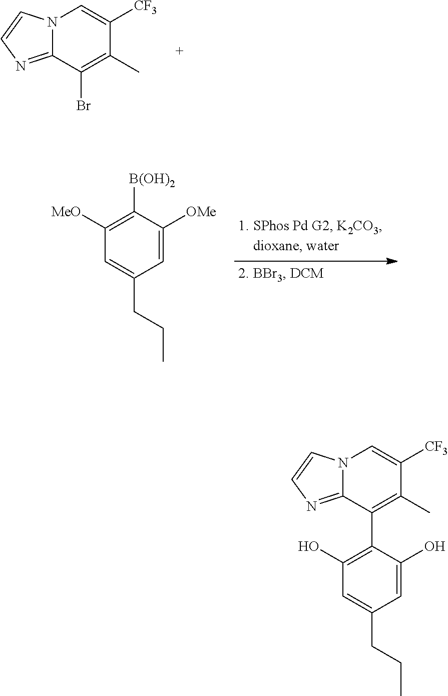 [SMILES: Cc1c(C(F)(F)F)cn2ccnc2c1Br ; CCCc1cc(OC)c(B(O)O)c(OC)c1 ; CCCc1cc(O)c(-c2c(C)c(C(F)(F)F)cn3ccnc23)c(O)c1]

Compound 16 was prepared from Intermediates 1 and 17.

¹H NMR (400 MHz, DMSO) δ 9.12 (s, 1H), 8.92 (s, 2H), 7.97 (d, J=1.4 Hz, 1H), 7.47 (d, J=1.4 Hz, 1H), 6.25 (s, 2H), 2.44 (t, J=7.7 Hz, 2H), 2.12 (s, 3H), 1.65-1.55 (m, 2H), 0.96 (t, J=7.5 Hz, 3H). MS (ESI): m/z 351.5 (M+1). HPLC purity: 96.6%.

### Compound 17: 2-(6-Chloro-7-methylimidazo[1,2-a]pyridin-8-yl)-5-pentylbenzene-1,3-diol

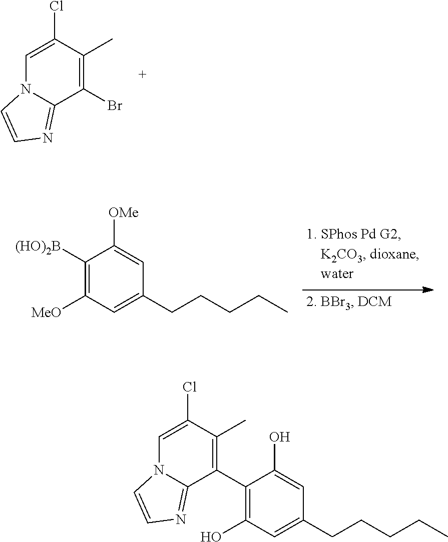 [SMILES: Cc1c(Cl)cn2ccnc2c1Br ; CCCCCc1cc(OC)c(B(O)O)c(OC)c1 ; CCCCCc1cc(O)c(-c2c(C)c(Cl)cn3ccnc23)c(O)c1]

Compound 17 was prepared from Intermediates 2 and 7.

¹H NMR (400 MHz, DMSO) δ 8.86 (s, 2H), 8.78 (s, 1H), 7.81 (d, J=1.2 Hz, 1H), 7.38 (d, J=1.0 Hz, 1H), 6.23 (s, 2H), 2.45 (t, J=7.6 Hz, 2H), 2.07 (s, 3H), 1.62-1.53 (m, 2H), 1.38-1.30 (m, 4H), 0.90 (t, J=7.0 Hz, 3H). MS (ESI): m/z 345.5 (M+1). HPLC purity: 99.1%.

### Compound 18: 2-(7-Cyclopropylimidazo[1,2-a]pyridin-8-yl)-5-propylbenzene-1,3-diol

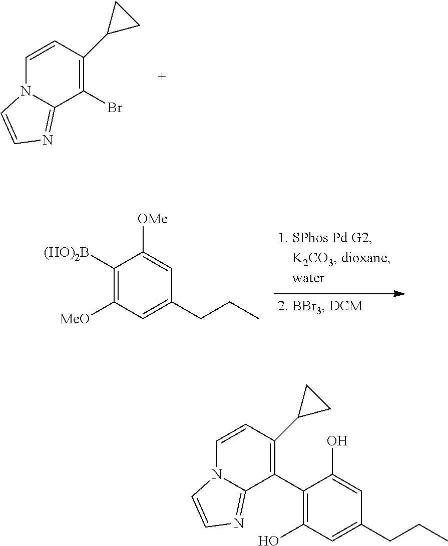 [SMILES: Brc1c(C2CC2)ccn2ccnc12 ; CCCc1cc(OC)c(B(O)O)c(OC)c1 ; CCCc1cc(O)c(-c2c(C3CC3)ccn3ccnc23)c(O)c1]

Compound 18 was prepared from Intermediates 1 and 18. The BBr₃ demethylation step was carried out at 50 °C in a sealed tube.

¹H NMR (400 MHz, DMSO) δ 8.80 (br s, 2H), 8.35 (d, J=7.0 Hz, 1H), 8.17 (s, 1H), 7.79 (d, J=1.3 Hz, 1H), 7.32 (d, J=1.1 Hz, 1H), 6.34 (d, J=7.2 Hz, 1H), 6.25-6.24 (m, 2H), 2.44 (t, J=7.6 Hz, 2H), 1.74-1.57 (m, 3H), 1.00-0.94 (m, 3H), 0.84-0.77 (m, 2H), 0.70-0.65 (m, 2H). MS (ESI): m/z 309.5 (M+1). HPLC purity: 91.9%.

### Compound 19: 5-Chloro-2-(6-chloro-7-methylimidazo[1,2-a]pyridin-8-yl)benzene-1,3-diol

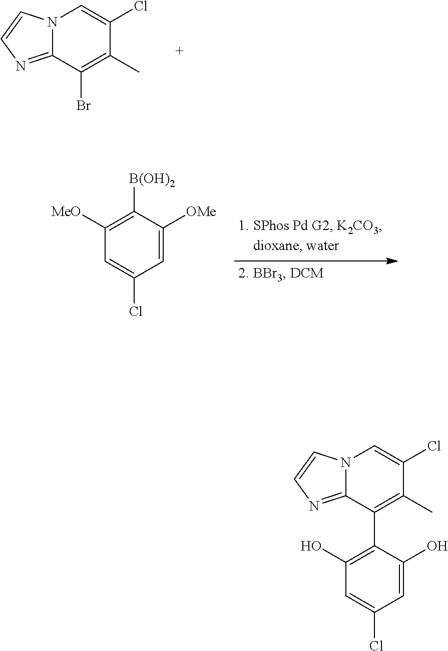 [SMILES: Cc1c(Cl)cn2ccnc2c1Br ; COc1cc(Cl)cc(OC)c1B(O)O ; Cc1c(Cl)cn2ccnc2c1-c1c(O)cc(Cl)cc1O]

Compound 19 was prepared from Intermediate 7 and (4-Chloro-2,6-dimethoxyphenyl)boronic acid which was sourced from Fluorochem Ltd.

¹H NMR (400 MHz, DMSO) δ 9.57 (s, 2H), 8.82 (s, 1H), 7.84 (d, J=1.2 Hz, 1H), 7.40 (d, J=1.2 Hz, 1H), 6.46 (s, 2H), 2.08 (s, 3H). MS (ESI): m/z 309.2 (M+1). HPLC purity: 98.1%.

### Compound 20: 2-(6-Chloro-7-methylimidazo[1,2-a]pyridin-8-yl)-5-(tert-pentyl)benzene-1,3-diol

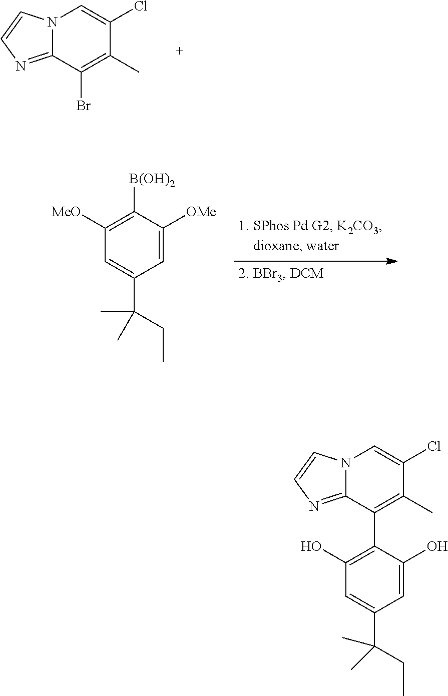 [SMILES: Cc1c(Cl)cn2ccnc2c1Br ; CCC(C)(C)c1cc(OC)c(B(O)O)c(OC)c1 ; CCC(C)(C)c1cc(O)c(-c2c(C)c(Cl)cn3ccnc23)c(O)c1]

Compound 20 was prepared from Intermediates 3 and 7.

¹H NMR (400 MHz, DMSO) δ 8.84-8.78 (m, 3H), (8.13 (s, 0.5H), 7.83 (s, 1H), 7.40 (s, 1H), 6.39 (s, 2H), 2.07 (s, 3H), 1.59 (q, J=7.3 Hz, 2H), 1.22 (s, 6H), 0.73 (t, J=7.5 Hz, 3H). MS (ESI): m/z 345.2 (M+1). HPLC purity: 97.4%. Isolated as a 0.5 equivalent formate salt.

### Compound 21: 2-(7-Methylimidazo[1,2-a]pyridin-8-yl)-5-(tert-pentyl)benzene-1,3-diol

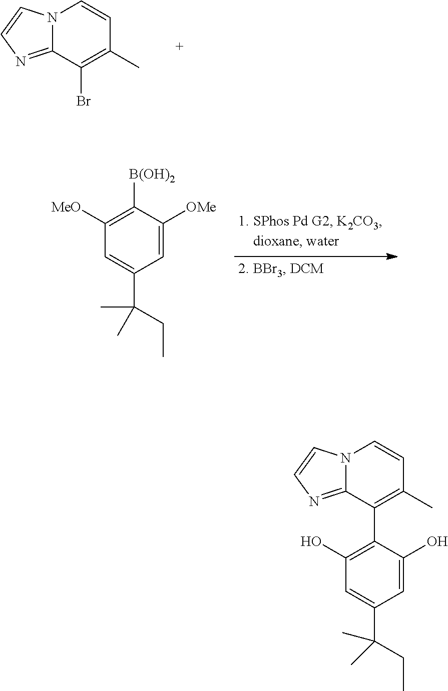 [SMILES: Cc1ccn2ccnc2c1Br ; CCC(C)(C)c1cc(OC)c(B(O)O)c(OC)c1 ; CCC(C)(C)c1cc(O)c(-c2c(C)ccn3ccnc23)c(O)c1]

Compound 21 was prepared from Intermediates 3 and 8.

¹H NMR (400 MHz, DMSO) δ 8.87 (br s, 2H), 8.38 (d, J=6.8 Hz, 1H), 8.14 (s, 1H), 7.84-7.82 (m, 1H), 7.35 (s, 1H), 6.81 (d, J=6.9 Hz, 1H), 6.38 (s, 2H), 2.06 (s, 3H), 1.59 (q, J=7.5 Hz, 2H), 1.23 (s, 6H), 0.73 (t, J=7.4 Hz, 3H). MS (ESI): m/z 311.2 (M+1). HPLC purity: 99.3%. Isolated as a 1.0 equivalent formate salt.

### Compound 25: 2-(6-Chloroimidazo[1,2-a]pyridin-8-yl)-5-propylbenzene-1,3-diol

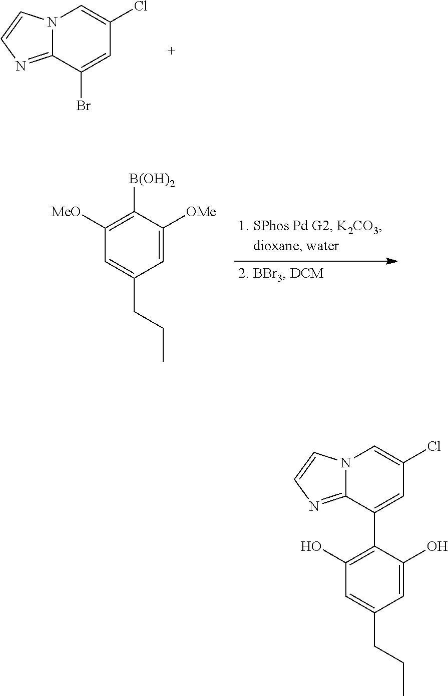 [SMILES: Clc1cc(Br)c2nccn2c1 ; CCCc1cc(OC)c(B(O)O)c(OC)c1 ; CCCc1cc(O)c(-c2cc(Cl)cn3ccnc23)c(O)c1]

Compound 25 was prepared from Intermediate 1 and 8-bromo-6-chloroimidazo[1,2-a]pyridine, which was sourced from Fluorochem Ltd.

¹H NMR (400 MHz, DMSO) δ 9.26 (br s, 1H), 8.15 (s, 1H), 8.80 (d, J=2.0 Hz, 1H), 7.93 (s, 1H), 7.52 (s, 1H), 7.11 (d, J=2.0 Hz, 1H), 6.26 (s, 2H), 2.43 (t, J=7.6 Hz, 2H), 1.64-1.54 (m, 2H), 0.94 (t, J=7.3 Hz, 3H). MS (ESI): m/z 303.2 (M+1). HPLC purity: 99.3%.

### Specific Synthetic Procedures

The following compounds were prepared using the specific routes described below.

### Compound 22: 5-Cyclopentyl-2-(7-methylimidazo[1,2-a]pyridin-8-yl)benzene-1,3-diol

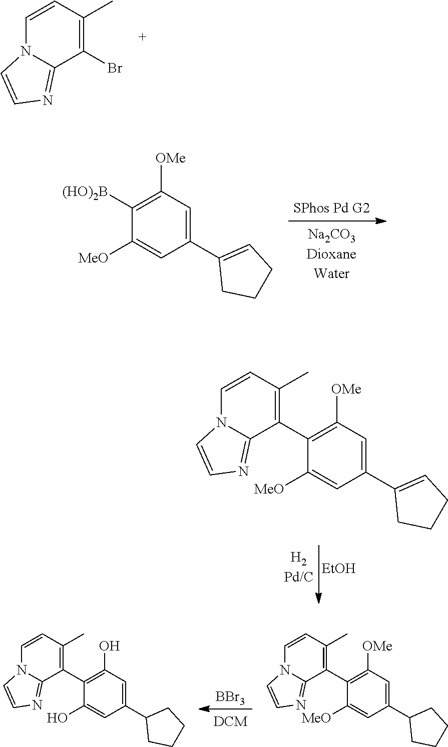

### Step 1: 8-(4-(Cyclopent-1-en-1-yl)-2,6-dimethoxyphenyl)-7-methylimidazo[1,2-a]pyridine

The title compound was prepared in a manner similar to the first step of Compound 1, using Intermediates 5 and 8. ¹H NMR (400 MHz, CDCl₃) δ 8.00 (d, J=6.8 Hz, 1H), 7.50 (d, J=1.2 Hz, 1H), 7.49 (d, J=1.2 Hz, 1H), 7.26 (s, 6H), 6.77 (s, 2H), 6.71 (d, J=6.8 Hz, 1H), 6.23-6.20 (m, 1H), 2.79-2.73 (m, 2H), 2.59-2.53 (m, 2H), 2.12 (s, 3H), 2.09-2.01 (m, 2H).

### Step 2: 8-(4-Cyclopentyl-2,6-dimethoxyphenyl)-7-methylimidazo[1,2-a]pyridine

8-(4-(Cyclopent-1-en-1-yl)-2,6-dimethoxyphenyl)-7-methylimidazo[1,2-a]pyridine (230 mg, 0.88 mmol) in ethanol (10 mL) was hydrogenated over palladium on carbon (10%, 50 mg, 0.0470 mmol) at room temperature and 1 atm overnight. The reaction mixture was filtered through Celite® and the solid washed with copious ethanol. The filtrate was concentrated in vacuo to give 8-(4-cyclopentyl-2,6-dimethoxyphenyl)-7-methylimidazo[1,2-a]pyridine (173 mg, 75%) as a yellow gum. ¹H NMR (400 MHz, CDCl₃) δ 8.05 (d, J=6.9 Hz, 1H), 7.58 (d, J=1.3 Hz, 1H), 7.52 (d, J=1.3 Hz, 1H), 6.81 (d, J=6.9 Hz, 1H), 6.58 (s, 2H), 3.70 (s, 6H), 3.09-3.00 (m, 1H), 2.15 (s, 3H), 2.14-2.08 (m, 2H), 1.86-1.81 (m, 2H), 1.76-1.65 (m, 4H).

### Step 3: 5-Cyclopentyl-2-(7-methylimidazo[1,2-a]pyridin-8-yl)benzene-1,3-diol

The title compound was prepared from 8-(4-cyclopentyl-2,6-dimethoxyphenyl)-7-methylimidazo[1,2-a]pyridine in a manner similar to the second step of Compound 1. ¹H NMR (400 MHz, DMSO) δ 8.89 (br s, 2H), 8.36 (d, J=6.8 Hz, 1H), 8.15 (s, 1H), 7.80 (d, J=1.3 Hz, 1H), 7.32 (d, J=1.3 Hz, 1H), 6.78 (d, J=6.8 Hz, 1H), 6.30 (s, 2H), 2.90-2.80 (m, 1H), 2.05 (s, 3H), 2.03-1.96 (m, 2H), 1.80-1.72 (m, 2H), 1.71-1.62 (m, 2H), 1.58-1.48 (m, 2H). MS (ESI): m/z 309.3 (M+1). HPLC purity: 95.1%. Isolated as a 1 equivalent formate salt.

### Compound 23: 2-(6-Cyclopropyl-7-methylimidazo[1,2-a]pyridin-8-yl)-5-propylbenzene-1,3-diol

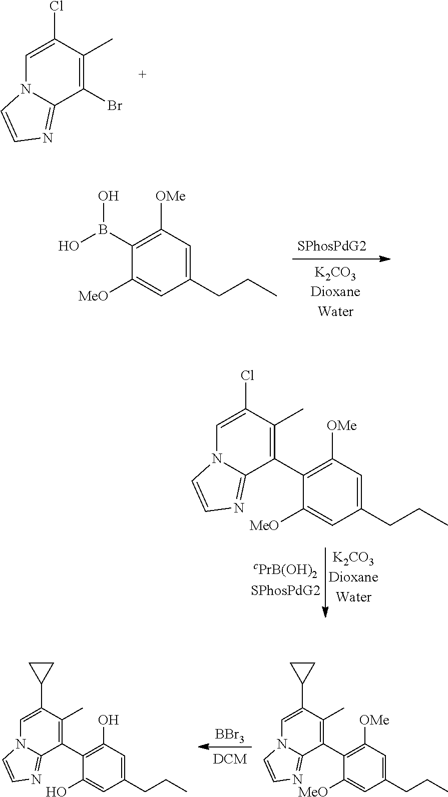

### Step 1: 6-Chloro-8-(2,6-dimethoxy-4-propylphenyl)-7-methylimidazo[1,2-a]pyridine

The title compound was prepared in a manner similar to the first step of Compound 1, using Intermediates 1 and 7. ¹H NMR (400 MHz, CDCl₃) δ 8.19 (s, 1H), 7.53 (d, J=1.2 Hz, 1H), 7.47 (d, J=1.2 Hz, 1H), 6.52 (s, 2H), 3.68 (s, 6H), 2.63 (t, J=7.7 Hz, 2H), 2.15 (s, 3H), 1.77-1.67 (m, 2H), 1.02 (t, J=7.3 Hz, 3H).

### Step 2: 6-Cyclopropyl-8-(2,6-dimethoxy-4-propylphenyl)-7-methylimidazo[1,2-a]pyridine

6-Chloro-8-(2,6-dimethoxy-4-propyl-phenyl)-7-methyl-imidazo[1,2-a]pyridine (200 mg, 0.580 mmol), cyclopropylboronic acid (249 mg, 2.90 mmol) and potassium carbonate (240 mg, 1.74 mmol) in 1,4-dioxane (3.5 mL) and water (1.5 mL) were degassed with nitrogen and treated with SPhos Pd G2 (42 mg, 0.0580 mmol). The reaction mixture was heated at 150 °C for 30 minutes then combined with that of a test reaction which had been run on 50 mg scale, diluted with ethyl acetate (50 mL), washed with water (50 mL), dried (phase separating paper) and concentrated in vacuo. The residue was purified by column chromatography on silica gel, eluting with 10-60% ethyl acetate in cyclohexane to give the title compound as an off-white solid (208 mg, 82%). ¹H NMR (400 MHz, CDCl₃) δ 7.82 (d, J=1.0 Hz, 1H), 7.46 (d, J=1.2 Hz, 1H), 7.41 (d, J=1.2 Hz, 1H), 6.52 (s, 2H), 3.67 (s, 6H), 2.64 (t, J=7.7 Hz, 2H), 2.17 (s, 3H), 1.90-1.83 (m, 1H), 1.78-1.67 (m, 2H), 1.02 (t, J=7.3 Hz, 3H), 0.97-0.91 (m, 2H), 0.68-0.63 (m, 2H).

### Step 3: 2-(6-Cyclopropyl-7-methylimidazo[1,2-a]pyridin-8-yl)-5-propylbenzene-1,3-diol

The title compound was prepared in a manner similar to the second step of Compound 2. ¹H NMR (400 MHz, MeOD) δ 8.53 (s, 0.5H), 8.41 (s, 1H), 7.95 (d, J=1.7 Hz, 1H), 7.64 (d, J=1.9 Hz, 1H), 6.41 (s, 2H), 2.56 (t, J=7.5 Hz, 2H), 2.40 (s, 3H), 2.09-2.00 (m, 1H), 1.76-1.66 (m, 2H), 1.12-1.07 (m, 2H), 1.03 (t, J=7.3 Hz, 3H), 0.83-0.78 (m, 2H). MS (ESI): m/z 323.0 (M+1). HPLC purity: 97.3%. Isolated as a 0.5 equivalent formate salt.

### Compound 24: 2-(6-Fluoro-7-methylimidazo[1,2-a]pyridin-8-yl)-5-(3,3,3-trifluoropropyl)benzene-1,3-diol

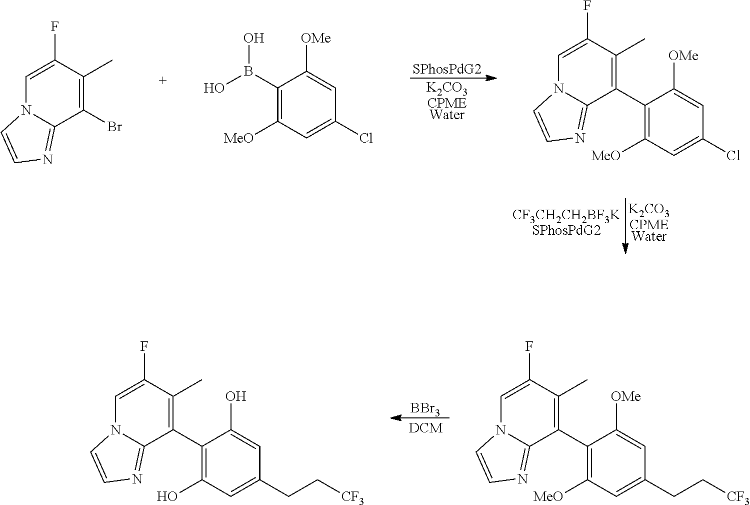

### Step 1: 8-(4-Chloro-2,6-dimethoxyphenyl)-6-fluoro-7-methylimidazo[1,2-a]pyridine

The title compound was prepared in a manner similar to the first step of Compound 1, using Intermediate 12 and (4-Chloro-2,6-dimethoxyphenyl)boronic acid (Fluorochem Ltd) and using CPME in place of dioxane. ¹H NMR (400 MHz, CDCl₃) δ 8.03 (d, J=4.4 Hz, 1H), 7.54 (d, J=1.2 Hz, 1H), 7.51 (d, J=1.2 Hz, 1H), 6.70 (s, 2H), 3.69 (s, 6H), 2.05 (d, J=2.3 Hz, 3H).

### Step 2: 8-(2,6-Dimethoxy-4-(3,3,3-trifluoropropyl)phenyl)-6-fluoro-7-methylimidazo[1,2-a]pyridine

8-(4-Chloro-2,6-dimethoxyphenyl)-6-fluoro-7-methylimidazo[1,2-a]pyridine (100 mg, 0.312 mmol), potassium trifluoro (3,3,3-trifluoropropyl) boranuide (95 mg, 0.468 mmol) and potassium carbonate (86 mg, 0.624 mmol) in cyclopentyl methyl ether (3 mL) and water (1 mL) were degassed with nitrogen, treated with SPhos Pd G2 (22 mg, 0.0312 mmol) and heated at 120 °C for 30 minutes in a microwave reactor. The reaction mixture was combined with that of a test reaction which has been run on 50 mg scale under identical conditions, poured into water (25 mL) and extracted with ethyl acetate (3×25 mL). The combined organic layers were dried (phase separating paper) and concentrated in vacuo. The residue was purified by column chromatography on 12 g silica gel, eluting with 10-100% ethyl acetate in cyclohexane to give the title compound as a cream solid (104 mg, 58%). ¹H NMR (400 MHz, CDCl₃) δ 8.02 (d, J=4.3 Hz, 1H), 7.54 (d, J=1.1 Hz, 1H), 7.50 (d, J=1.2 Hz, 1H), 6.52 (s, 2H), 3.69 (s, 6H), 2.95-2.89 (m, 2H), 2.54-2.41 (m, 2H), 2.05 (d, J=2.3 Hz, 3H).

### Step 3: 2-(6-Fluoro-7-methylimidazo[1,2-a]pyridin-8-yl)-5-(3,3,3-trifluoropropyl)-benzene-1,3-diol

The title compound was prepared in a manner similar to the second step of Compound 1. ¹H NMR (400 MHz, DMSO) δ 9.01 (br s, 2H), 8.67 (d, J=4.9 Hz, 1H), 8.14 (s, 1H), 7.83 (d, J=1.1 Hz, 1H), 7.38 (d, J=1.2 Hz, 1H), 6.30 (s, 2H), 2.74-2.68 (m, 2H), 2.62-2.53 (m, 2H), 1.99 (d, J=2.3 Hz, 3H). MS (ESI): m/z 355.2 (M+1). HPLC purity: 99.5%. Isolated as 1 equivalent formate salt.

### Example 2: Evaluation of the Anticonvulsive Properties of Compounds Using the Mouse Maximal Electroshock Seizure Threshold Model Using Minimal Sample Sizes (Mini MEST)

The maximal electroshock seizure threshold (MEST) test is widely utilized preclinically to evaluate pro- or anti-convulsive properties of test compounds (Löscher et al., 1991).

In the MEST test the ability of a drug to alter the seizure threshold current required to induce hind limb tonic extensor convulsions is measured according to an “up and down” method of shock titration (Kimball et al., 1957). An increase in seizure threshold is indicative of an anticonvulsive effect. Antiepileptic drugs including diazepam with clinically proven efficacy against generalised tonic-clonic seizures, exhibit anticonvulsive properties in this test in the mouse (Löscher et al., 1991).

Conversely, a reduction in seizure threshold is indicative of a proconvulsive effect as observed with known convulsive agents such as picrotoxin.

The ability of a test compound to alter the stimulus intensity, expressed as current (mA), required to induce the presence of tonic hind limb extensor convulsions, is assessed in the MEST. The outcome of the presence (+) or absence (0) of tonic hind limb extensor convulsions observed from a current to produce tonic hind limb extension in 50% of animals in the treatment group (CC₅₀) determines the seizure threshold for the treatment group and the effects were then compared to the CC₅₀ of the vehicle control group.

In the mini-MEST test, a lower n number is used.

### General Method

### Mice:

Male C57BL/6J mice were housed in groups of 6. Mice were kept under standard conditions (12 hour light cycle; light intensity of 25-75 lux) and fed a diet of certified rodent CR 14% protein rodent diet (LabDiet® 4CR4). Mice were 8-9 weeks old at the start of the study.

### Study Details:

Naïve mice were acclimatised to the procedure room in their home cages for up to 7 days, with food and water available ad libitum.

All animals were weighed at the beginning of the study and randomly assigned to treatment groups based on a mean distribution of body weight across groups. All animals were dosed at 10 mL/kg via intraperitoneal (i.p) injection, with either vehicle, test compound at 5-50 mg/kg, or diazepam at 2.5 mg/kg.

Animals were individually assessed for the production of a tonic hind limb extensor convulsion at 30 min post-dose for vehicle, 15-30 min post-dose for test compound (dependent on compound) and 30 min post-dose for diazepam, from a single electroshock.

The first animal within a treatment group was given a shock at the expected or estimated CC₅₀ current. For subsequent animals, the current was lowered or raised depending on the convulsions outcome from the preceding animal in log scale intervals.

Data generated from each treatment group were used to calculate the CC₅₀±SEM values for the treatment group.

### Test Compounds:

Vehicle: (5% ethanol, 10% Solutol® (Kolliphor® HS 15), 85% saline). 1 mL of ethanol, 2 mL of Solutol™ were warmed to 60 °C, in 17 mL of 0.9% saline.

Positive control: diazepam was used at 2.5 mg/kg in a 1:2:17 ethanol:Solutol™:saline formulation (as used for vehicle).

Test compounds: 5-50 mg/kg in a 1:2:17 ethanol:Solutol®:saline formulation (as used for vehicle).

### Sample Collection:

Each animal was humanely killed immediately after production of a convulsion by concussion of the brain from striking the cranium, followed by the confirmation of permanent cessation of the circulation from decapitation under The Humane Killing of Animals under Schedule 1 to the Animals (Scientific Procedures) Act 1986. Terminal blood and brain collection were performed following decapitation.

Blood was collected in Lithium-heparin or K2EDTA tubes and centrifuged at 4 °C for 10 minutes at 1500×g. The resulting plasma was removed (100 μL) and split into 2 aliquots of 0.5 mL Eppendorf tubes containing 100 μL of ascorbic acid (100 mg/mL) for stabilisation. Brains were removed, washed in saline and halved. Each half was placed into separate 2 mL screw cap cryovials, weighed and frozen on cardice.

### Results

Table 1 to Table 19 show the results for each study group.

TABLE 1
Evaluation study group 1

| | | Test time | | | |
|---|---|---|---|---|---|
| | Dose | post dose | | | Change from |
| Compound | (mg/kg) | (min) | N | CC₅₀ +/− SEM | vehicle (%) |
| Vehicle | — | 30 | 6 | 31.0 ± 2.6 | — |
| Diazepam | 2.5 | 30 | 6 | 128.0 ± 5.2 | 313% |
| Compound 1 | 30 | 30 | 6 | 92.9 ± 1.1 | 200% |

TABLE 2
Evaluation study group 2

| | | Test time | | | |
|---|---|---|---|---|---|
| | Dose | post dose | | | Change from |
| Compound | (mg/kg) | (min) | N | CC₅₀ +/− SEM | vehicle (%) |
| Vehicle | — | 30 | 6 | 25.5 ± 0.9 | — |
| Diazepam | 2.5 | 30 | 6 | 121.0 ± 0.6 | 375% |
| Compound 2 | 30 | 30 | 6 | >131.0 | 414% |

TABLE 3
Evaluation study group 3

| | | Test time | | | |
|---|---|---|---|---|---|
| | Dose | post dose | | | Change from |
| Compound | (mg/kg) | (min) | N | CC₅₀ +/− SEM | vehicle (%) |
| Vehicle | — | 30 | 6 | 27.0 ± 0.1 | — |
| Diazepam | 2.5 | 30 | 6 | 106.5 ± 1 4.5 | 294% |
| Compound 2 | 10 | 15 | 6 | 67.5 ± 2.6 | 150% |

TABLE 4
Evaluation study group 4

| | | Test time | | | |
|---|---|---|---|---|---|
| | Dose | post dose | | | Change from |
| Compound | (mg/kg) | (min) | N | CC₅₀ +/− SEM | vehicle (%) |
| Vehicle | — | 30 | 6 | 26.8 ± 1.1 | — |
| Diazepam | 2.5 | 30 | 6 | 99.5 ± 1.9 | 272% |
| Compound 2 | 10 | 15 | 6 | 65.8 ± 1.9 | 145% |
| Compound 2 | 10 | 30 | 6 | 35.3 ± 1.1 | 32% |
| Compound 2 | 10 | 60 | 6 | 56.0 ± 1.6 | 109% |
| Compound 3 | 30 | 30 | 6 | 35.3 ± 1.1 | 32% |

TABLE 5
Evaluation study group 5

| | | Test time | | | |
|---|---|---|---|---|---|
| | Dose | post dose | | | Change from |
| Compound | (mg/kg) | (min) | N | CC₅₀ +/− SEM | vehicle (%) |
| Vehicle | — | 30 | 6 | 29.4 ± 1.6 | — |
| Diazepam | 2.5 | 30 | 6 | 84.7 ± 1.6 | 188% |
| Compound 4 | 30 | 30 | 6 | 70.5 ± 1.1 | 140% |
| Compound 5 | 30 | 30 | 6 | <33.0 | <12% |
| Compound 6 | 30 | 30 | 6 | 49.9 ± 1.9 | 70% |

TABLE 6
Evaluation study group 6

| | | Test time | | | |
|---|---|---|---|---|---|
| | Dose | post dose | | | Change from |
| Compound | (mg/kg) | (min) | N | CC₅₀ +/− SEM | vehicle (%) |
| Vehicle | — | 30 | 6 | 33.0 ± 1.9 | — |
| Diazepam | 2.5 | 30 | 6 | 161.4 ± 1.1 | 390% |
| Compound 4 | 50* | 30 | 6 | 80.9 ± 1.1 | 145% |
| Compound 4 | 50* | 60 | 6 | >131.0 | >297% |

*compound was dosed p.o. via oral gavage

TABLE 7
Evaluation study group 7

| | | Test time | | | |
|---|---|---|---|---|---|
| | Dose | post dose | | | Change from |
| Compound | (mg/kg) | (min) | N | CC₅₀ +/− SEM | vehicle (%) |
| Vehicle | — | 30 | 6 | 30.7 ± 0.0 | — |
| Diazepam | 2.5 | 30 | 6 | 122.4 ± 1.1 | 298% |
| Compound 4 | 50* | 120 | 6 | 61.4 ± 1.1 | 100% |
| Compound 4 | 50* | 240 | 6 | 61.4 ± 3.7 | 100% |
| Compound 19 | 50* | 30 | 6 | 57.3 ± 1.9 | 86% |

*compound was dosed p.o. via oral gavage

TABLE 8
Evaluation study group 8

| | | Test time | | | |
|---|---|---|---|---|---|
| | Dose | post dose | | | Change from |
| Compound | (mg/kg) | (min) | N | CC₅₀ +/− SEM | vehicle (%) |
| Vehicle | — | 30 | 6 | 25.6 ± 1.6 | — |
| Diazepam | 2.5 | 30 | 6 | 150.6 ± 1.9 | 489% |
| Compound 4 | 25* | 15 | 6 | 80.9 ± 3.7 | 216% |
| Compound 4 | 25* | 30 | 6 | 92.9 ± 1.1 | 263% |
| Compound 4 | 25* | 60 | 6 | 70.5 ± 1.1 | 176% |

*compound was dosed p.o. via oral gavage

TABLE 9
Evaluation study group 9

| | | Test time | | | |
|---|---|---|---|---|---|
| | Dose | post dose | | | Change from |
| Compound | (mg/kg) | (min) | N | CC₅₀ +/− SEM | vehicle (%) |
| Vehicle | — | 30 | 6 | 25.0 ± 1.9 | — |
| Diazepam | 2.5 | 30 | 6 | 185.3 ± 1.1 | 641% |
| Compound 4 | 25* | 30 | 12 | 53.5 ± 3.4 | 114% |

*compound was dosed p.o. via oral gavage

TABLE 10
Evaluation study group 10

| | | Test time | | | |
|---|---|---|---|---|---|
| | Dose | post dose | | | Change from |
| Compound | (mg/kg) | (min) | N | CC₅₀ +/− SEM | vehicle (%) |
| Vehicle | — | 30 | 6 | 25.0 ± 1.9 | — |
| Diazepam | 2.5 | 30 | 6 | 173.0 ± 1.9 | 592% |
| Compound 4 | 25* | 30 | 6 | 106.6 ± 1.1 | 327% |
| Compound 24 | 25* | 30 | 6 | 92.9 ± 1.1 | 272% |
| Compound 24 | 50* | 30 | 6 | >131.0 | >424% |

*compound was dosed p.o. via oral gavage

TABLE 11
Evaluation study group 11

| | | Test time | | | |
|---|---|---|---|---|---|
| | Dose | post dose | | | Change from |
| Compound | (mg/kg) | (min) | N | CC₅₀ +/− SEM | vehicle (%) |
| Vehicle | — | 30 | 6 | 26.8 ± 1.1 | — |
| Diazepam | 2.5 | 30 | 6 | 131.2 ± 1.9 | 390% |
| Compound 7 | 30 | 30 | 6 | 53.5 ± 1.1 | 100% |
| Compound 8 | 30 | 30 | 6 | 65.8 ± 1.9 | 145% |

TABLE 12
Evaluation study group 12

| | | Test time | | | |
|---|---|---|---|---|---|
| | Dose | post dose | | | Change from |
| Compound | (mg/kg) | (min) | N | CC₅₀ +/− SEM | vehicle (%) |
| Vehicle | — | 30 | 6 | 23.3 ± 1.1 | — |
| Diazepam | 2.5 | 30 | 6 | 140.6 ± 1.1 | 503% |
| Compound 9 | 30 | 30 | 6 | 58.6 ± 1.6 | 152% |
| Compound 11 | 30 | 30 | 6 | 40.6 ± 1.1 | 74% |

TABLE 13
Evaluation study group 13

| | | Test time | | | |
|---|---|---|---|---|---|
| | Dose | post dose | | | Change from |
| Compound | (mg/kg) | (min) | N | CC₅₀ +/− SEM | vehicle (%) |
| Vehicle | — | 30 | 6 | 25.6 ± 1.6 | — |
| Diazepam | 2.5 | 30 | 6 | 122.4 ± 1.1 | 379% |
| Compound 10 | 30 | 30 | 6 | <33.0 ± 0.0 | <29% |

TABLE 14
Evaluation study group 14

| | | Test time | | | |
|---|---|---|---|---|---|
| | Dose | post dose | | | Change from |
| Compound | (mg/kg) | (min) | N | CC₅₀ +/− SEM | vehicle (%) |
| Vehicle | — | 30 | 6 | 23.3 ± 1.1 | — |
| Diazepam | 2.5 | 30 | 6 | 128.0 ± 34 | 462% |
| Compound 12 | 30 | 30 | 6 | 57.3 ± 1.9 | 145% |

TABLE 15
Evaluation study group 15

| | | Test time | | | |
|---|---|---|---|---|---|
| | Dose | post dose | | | Change from |
| Compound | (mg/kg) | (min) | N | CC₅₀ +/− SEM | vehicle (%) |
| Vehicle | — | 30 | 6 | 25.6 ± 1.6 | — |
| Diazepam | 2.5 | 30 | 6 | 99.5 ± 1.9 | 289% |
| Compound 11 | 30 | 30 | 6 | 92.9 ± 1.1 | 263% |
| Compound 13 | 30 | 30 | 6 | 92.9 ± 1.1 | 263% |
| Compound 18 | 30 | 30 | 6 | 80.9 ± 1.1 | 216% |
| Compound 22 | 30 | 30 | 6 | 57.3 ± 1.9 | 124% |

TABLE 16
Evaluation study group 16

| | | Test time | | | |
|---|---|---|---|---|---|
| | Dose | post dose | | | Change from |
| Compound | (mg/kg) | (min) | N | CC₅₀ +/− SEM | vehicle (%) |
| Vehicle | — | 30 | 6 | 21.8 ± 1.9 | — |
| Diazepam | 2.5 | 30 | 6 | 92.9 ± 3.7 | 327% |
| Compound 14 | 30 | 30 | 6 | <33.0 | <52% |
| Compound 15 | 30 | 30 | 6 | 35.3 ± 1.1 | 62% |
| Compound 23 | 30 | 30 | 6 | <33.0 | <52% |

TABLE 17
Evaluation study group 17

| | | Test time | | | |
|---|---|---|---|---|---|
| | Dose | post dose | | | Change from |
| Compound | (mg/kg) | (min) | N | CC₅₀ +/− SEM | vehicle (%) |
| Vehicle | — | 30 | 6 | 24.4 ± 1.6 | — |
| Diazepam | 2.5 | 30 | 6 | 131.2 ± 1.9 | 437% |
| Compound 16 | 30 | 30 | 6 | <33.0 | <35% |
| Compound 17 | 30 | 30 | 6 | 80.9 ± 10.8 | 231% |

TABLE 18
Evaluation study group 18

| | | Test time | | | |
|---|---|---|---|---|---|
| | Dose | post dose | | | Change from |
| Compound | (mg/kg) | (min) | N | CC₅₀ +/− SEM | vehicle (%) |
| Vehicle | — | 30 | 6 | 25.6 ± 1.6 | — |
| Diazepam | 2.5 | 30 | 6 | 128.2 ± 1.6 | 401% |
| Compound 20 | 30 | 30 | 6 | 40.6 ± 1.1 | 59% |
| Compound 21 | 30 | 30 | 6 | <33.0 | NA % |

TABLE 19
Evaluation study group 19

| | | Test time | | | |
|---|---|---|---|---|---|
| | Dose | post dose | | | Change from |
| Compound | (mg/kg) | (min) | N | CC₅₀ +/− SEM | vehicle (%) |
| Vehicle | — | 30 | 6 | 26.8 ± 1.1 | — |
| Diazepam | 2.5 | 30 | 6 | 150.6 ± 1.9 | 462% |
| Compound 18 | 50* | 30 | 6 | 49.9 ± 1.9 | 86% |

*compound was dosed p.o. via oral gavage

### Conclusions

The results shown in Table 1 to Table 19 demonstrate that certain test compounds raise CC₅₀ values compared to vehicle. This data demonstrates that certain test compounds display anticonvulsant activity in a mouse model of generalised seizure. Thus, the test compounds may be useful in medical treatment, such as in the treatment of seizure.

### Example 3: Evaluation of the Anticonvulsive Properties of Compounds Using the Mouse Maximal Electroshock Seizure Threshold Model (MEST)

The maximal electroshock seizure threshold (MEST) test is widely utilized preclinically to evaluate pro- or anti-convulsive properties of test compounds (Löscher et al., 1991).

In the MEST test the ability of a drug to alter the seizure threshold current required to induce hind limb tonic extensor convulsions is measured according to an “up and down” method of shock titration (Kimball et al., 1957). An increase in seizure threshold is indicative of an anticonvulsive effect. Antiepileptic drugs including diazepam with clinically proven efficacy against generalised tonic-clonic seizures, exhibit anticonvulsive properties in this test in the mouse (Löscher et al., 1991).

Conversely, a reduction in seizure threshold is indicative of a proconvulsive effect as observed with known convulsive agents such as picrotoxin.

The ability of a test compound to alter the stimulus intensity, expressed as current (mA), required to induce the presence of tonic hind limb extensor convulsions, is assessed in the MEST. The outcome of the presence (+) or absence (0) of tonic hind limb extensor convulsions observed from a current to produce tonic hind limb extension in 50% of animals in the treatment group (CC₅₀) determines the seizure threshold for the treatment group and the effects were then compared to the CC₅₀ of the vehicle control group.

### General Method

### Mice:

Male C57BL/6J mice were housed in groups of 6. Mice were kept under standard conditions (12 hour light cycle; light intensity of 25-75 lux) and fed a diet of certified rodent CR 14% protein rodent diet (LabDiet® 5CR4). Mice were 8-9 weeks old at the start of the study.

### Study Details:

Naïve mice were acclimatised to the procedure room in their home cages for up to 7 days, with food and water available ad libitum.

All animals were weighed at the beginning of the study and randomly assigned to treatment groups (n=12/group) based on a mean distribution of body weight across groups. All animals were dosed at 10 ml/kg via:
a. intraperitoneal (i.p) injection, with either vehicle (1:2:17 ethanol:Solutol® (Kolliphor® HS 15:saline); or 1:1:18 ethanol:Kolliphor®EL:saline), test compound in vehicle, sodium valproate in vehicle, or diazepam in vehicle; or
b. oral gavage (p.o.), with either vehicle (0.5% HPMC and 0.1% Tween 80), or test compound in vehicle.

Animals were individually assessed for the production of a tonic hind limb extensor convulsion at 30 min post-dose for vehicle, 15-60 min post-dose for test compound (dependent on compound), 30 min post-dose for sodium valproate and 30 min post-dose for diazepam, from a single electroshock.

The first animal within a treatment group was given a shock at the expected or estimated CC₅₀ current. For subsequent animals, the current was lowered or raised depending on the convulsions outcome from the preceding animal in 5 mA intervals.

Data generated from each treatment group were used to calculate the CC₅₀±SEM values for the treatment group.

### Vehicle Preparation:

Vehicle=1:2:17 ethanol:Solutol® (Kolliphor® HS 15):saline: 1 mL of ethanol and 2 mL of solutol were warmed to 60 °C and 17 mL of 0.9% saline was added slowly.

Vehicle=1:1:18 ethanol:Kolliphor®EL:saline: 1.6 mL of ethanol and 1.6 mL of Kolliphor®EL (Cremaphor®) were warmed to 60 °C and 28.8 mL of 0.9% saline was added slowly.

Vehicle=0.5% HPMC and 0.1% Tween 80:0.5% HPMC and 0.1% Tween 80 in water.

### Sample Collection:

Each animal was humanely killed immediately after production of a convulsion by concussion of the brain from striking the cranium, followed by the confirmation of permanent cessation of the circulation from decapitation under The Humane Killing of Animals under Schedule 1 to the Animals (Scientific Procedures) Act 1986. Terminal blood and brain collection were performed following decapitation.

Blood was collected in K2-EDTA tubes (BD Microtainer, BD, USA) and centrifuged at 4 °C for 10 minutes at 1500×g. The resulting plasma was removed (100 μL) and split into 2 aliquots of 0.5 mL Eppendorf tubes containing 100 μL of ascorbic acid (100 mg/mL) for stabilisation. Brains were removed and halved. Each half was placed into separate 5 mL Eppendorf tubes, weighed and frozen on cardice.

### Results

Table 20 to Table 23 show the results for the tested compounds.

TABLE 20
MEST results for Compound 2 (n = 12)

| | | | Test time | | |
|---|---|---|---|---|---|
| | | | post | | Change |
| | Dose | | dose | | from |
| Compound | (mg/kg) | Route | (min) | CC₅₀ +/− SEM (mA) | vehicle (%) |
| Vehicle (1:2:17 | — | i.p. | 30 | 23.3 ± 0.3 | — |
| ethanol:Solutol ®:saline) | | | | | |
| Diazepam | 2.5 | i.p. | 30 | 125.8 ± 3.9 | 439%^{###} |
| Sodium Valproate | 150 | i.p. | 30 | 104.2 ± 1.0 | 346%^{###} |
| Compound 2 | 1 | i.p. | 15 | 26.0 ± 0.4 | 11%^{###} |
| Compound 2 | 5 | i.p. | 15 | 29.4 ± 0.9 | 26%^{###} |
| Compound 2 | 30 | i.p. | 30 | 38.2 ± 1.5 | 64%^{###} |
| Compound 2 | 60 | i.p. | 30 | 59.5 ± 2.6 | 155%^{###} |
| Vehicle (HPMC) | — | p.o. | 30 | 23.0 ± 0.1 | — |
| Compound 2 | 10 | p.o. | 30 | 27.0 ± 0.7 | 17%*** |
| Compound 2 | 50 | p.o. | 30 | 38.2 ± 0.5 | 66%*** |

^{###}p < 0.001 when compared to the vehicle (1:2:17 ethanol:Kolliphor@ HS 15:saline) group;
***p < 0.001 when compared to the vehicle (HPMC) group

TABLE 21
MEST results for Compound 4 (n = 12)

| | | | Test time | | |
|---|---|---|---|---|---|
| | Dose | | post dose | CC₅₀ +/− SEM | Change from |
| Compound | (mg/kg) | Route | (min) | (mA) | vehicle (%) |
| Vehicle (1:1:18 | — | i.p. | 30 | 25.0 ± 2.0 | — |
| ethanol:Kolliphor ®EL:saline) | | | | | |
| Diazepam | 2.5 | i.p. | 30 | 126.5 ± 2.1 | 406%^{###} |
| Sodium Valproate | 150 | i.p. | 30 | 139.2 ± 2.1 | 457%^{###} |
| Vehicle (HPMC) | — | p.o. | 30 | 20.8 ± 1.9 | — |
| Compound 4 | 5 | p.o. | 15 | 23.5 ± 2.1 | 11% |
| Compound 4 | 25 | p.o. | 15 | 27.5 ± 1.3 | 32%** |
| Compound 4 | 50 | p.o. | 30 | 92.5 ± 3.2 | 344%*** |
| Compound 4 | 100 | p.o. | 30 | 200.0 ± 3.7 | 860%*** |

^{###}p < 0.001 when compared to the vehicle (1:1:18 ethanol:Kolliphor ® EL:saline) group;
**p < 0.01 or
***p < 0.001 when compared to the vehicle (HPMC) group

TABLE 22
MEST results for Compound 4 (n = 12)

| | | | Test time | CC₅₀ +/− | |
|---|---|---|---|---|---|
| | Dose | | post dose | SEM | Change from |
| Compound | (mg/kg) | Route | (min) | (mA) | vehicle (%) |
| Vehicle | — | p.o. | 30 | 26.3 ± 1.1 | — |
| (HPMC) | | | | | |
| Diazepam | 2.5 | i.p. | 30 | 197.5 ± 3.2 | 652%*** |
| Sodium | 150 | i.p. | 30 | >300.0 | >1043%*** |
| Valproate | | | | | |
| Compound 4 | 10 | p.o. | 30 | 22.5 ± 0.3 | 14% |
| Compound 4 | 25 | p.o. | 30 | 60.8 ± 2.9 | 132%*** |
| Compound 4 | 50 | p.o. | 30 | 125.0 ± 33.4 | 376%** |

**p < 0.01 or
***p < 0.001 when compared to the vehicle (HPMC) group.

TABLE 23
MEST results for Compound 11 (n = 12)

| | | | Test time | CC₅₀ +/− | Change |
|---|---|---|---|---|---|
| | Dose | | post dose | SEM | from |
| Compound | (mg/kg) | Route | (min) | (mA) | vehicle (%) |
| Diazepam | 2.5 | i.p. | 30 | 165.8 ± 1.4 | 526%*** |
| Sodium | 150 | i.p. | 30 | 215.8 ± 30.1 | 714%*** |
| Valproate | | | | | |
| Vehicle | — | p.o. | 30 | 26.5 ± 1.0 | — |
| (HPMC) | | | | | |
| Compound 11 | 5 | p.o. | 15 | 25.8 ± 1.9 | −3% |
| Compound 11 | 25 | p.o. | 15 | 37.5 ± 2.2 | 42%*** |
| Compound 11 | 50 | p.o. | 30 | 100.8 ± 3.9 | 281%*** |
| Compound 11 | 100 | p.o. | 30 | 250.0 ± 3.7 | 843%*** |

***p < 0.001 when compared to the vehicle (HPMC) group

### Conclusions

The results shown in Table 20 to Table 23 demonstrate that the test compounds raise CC₅₀ values compared to vehicle. In addition, a dose response was observed in all cases. These data demonstrate that the test compounds display anticonvulsant activity in a mouse model of generalised seizure. Thus, the test compounds may be useful in medical treatment, such as in the treatment of seizure.

### Example 4: Evaluation of the Anticonvulsive Properties of Compounds Using the Mouse 6 Hz (44 mA) Model of Partial Seizures

The 6 Hz (44 mA) model of partial seizures, which detects anticonvulsant activity, follows that described by Brown et al. 1953.

In the 6 Hz test, the ability of a compound to reduce the severity and number of seizures is assessed by measuring protection from electrically-evoked forelimb clonus. A decrease in forelimb clonus score is indicative of protection from seizure and an anticonvulsive effect.

When conducted at a 44 mA stimulus intensity in mice, the 6 Hz test can differentiate compounds with potential for the treatment of focal-onset seizures that are often resistant to current anti-epileptic drugs. Antiepileptic drugs such as cannabidiol exhibit anticonvulsive effects in this test (Klein et al., 2017).

### General Method

### Mice:

Male RjOrl: Swiss mice (Janvier Labs, 53940 Le Genest-Saint-Isle, France), 5 weeks old, weighing 27-38 g at the beginning of the experiment. Animals were acclimatized to the Test Facility for at least 5 days after delivery and randomly housed in groups of 5 to 8 in macrolon cages on wood litter with free access to food and water.

The animal house was maintained under artificial lighting (12 hours) between 7:00 and 19:00 in a controlled ambient temperature of 22±2 °C and relative humidity between 30-70%.

### Study Details:

Before transcorneal stimulation, a drop of tetracaine solution (1%) was applied on each eye of the mouse for local anesthesia. Between 1-10 minutes later and at the designated pre-treatment (see below), the mice were administered a rectangular current (44 mA, rectangular pulse: 0.2 ms pulse width, 3 s duration, (6 Hz) via corneal electrodes connected to a constant current shock generator (Ugo Basile: type 7801).

The results for the number of seizures as reflected by forelimb clonus were recorded immediately after current administration. Forelimb clonus was scored as absent (0=no forelimb clonus), mild (1=clonus with one forelimb) and strong (2=clonus with both forelimbs). The experimenters were blinded to the treatment.

Test Compound was Administered at 10 mL/Kg Via:
a. intraperitoneally (i.p) at 30 mg/kg, 15 minutes before the test and at 10, 60 and 100 mg/kg 30 minutes before the test and was compared with the corresponding vehicle (5% ethanol, 10% Solutol® (Kolliphor® HS 15), 85% physiological saline); or
b. oral gavage (p.o.) at 25 mg/kg, 15 minutes before the test and at 50 and 100 mg/kg 30 minutes before the test and at 200 mg/kg 60 minutes before the test and was compared with vehicle (0.5% HPMC and 0.1% Tween 80).

Valproate (200 mg/kg i.p.), administered intraperitoneally 30 minutes before the test, was used as reference substance and was compared with the corresponding vehicle (5% ethanol, 10% Kolliphor HS15, 85% physiological saline).

The administration volume was 10 mL/kg.

Due to the number of animals tested, the experiment was divided in 2 sub-experiments on 2 consecutive days with equivalent numbers of mice per group and per sub-experiment.

### Sample Collection:

The euthanasia and tissue collection method, which was designed to minimize animal suffering and to ensure good quality of biological samples, was adapted from basic procedures commonly used in studies performed in rodents.

At the end of test, the mice treated with the test substance were placed under isoflurane anaesthesia (5% for induction and 2% for maintenance, under 100% O2). Approximately 500 μL of blood were collected by cardiac puncture using a sterile disposable syringe. The blood samples were immediately transferred into pre-labelled tubes containing K2-EDTA. After sealing each tube, the blood samples were manually agitated and stored on ice until centrifugation (within 30 minutes of sampling). The samples were centrifuged at +4 °C, at 1500 g, for 10 minutes. Once separated, the resulting plasma was stabilised immediately with 100 mg/mL ascorbic acid (aq) in a 1:1 v/v ratio (2 aliquots of 100 μL). Ascorbic acid was prepared fresh on the day of use. The samples were stored at −80 °C until shipment.

Each animal was humanely killed immediately after blood collection in compliance with Council Directive No. 2010/63/UE of Sep. 22, 2010 on the protection of animals used for scientific purposes and French decree No. 2013-118 of Feb. 1, 2013 on the protection of animals Animal Health regulations. The brain hemispheres were dissected, weighed, placed in separate pre-labelled vials and snap frozen on liquid nitrogen. The vials were stored upright at approximately −80 °C and protected from light until transport to the bioanalysis site for analysis.

### Statistical Analysis:

Data were discrete values so considered to be non-normally distributed based on validation data, therefore non-parametric tests were utilized. Quantitative data (scores) with the test substance were analyzed by comparing test compound-treated groups with the 1:2:17 vehicle control using Kruskal-Wallis test with Dunn's multiple comparisons test when the Kruskal-Wallis test was significant. Quantitative data with the reference substance were analyzed using Mann-Whitney U test as compared to saline vehicle.

### Results

Table 24 to Table 26 show the results for the tested compounds.

TABLE 24
6 Hz Partial Seizure Test Results for Compound 2 (n = 15)

| | | Test | | | |
|---|---|---|---|---|---|
| | | time | Mean | Change | Change |
| | Dose | post | Forelimb | from | from |
| | (mg/ | dose | Clonus +/− | vehicle | vehicle |
| Compound | kg) | (min) | SEM | 1) (%) | 2 (%) |
| Vehicle 1 | — | 30 | 1.0 ± 0.1 | — | — |
| (saline) | | | | | |
| Vehicle 2 (1:2:17 | — | 30 | 0.9 ± 0.2 | −10% | — |
| ethanol:Kolliphor | | | | | |
| HS15; saline) | | | | | |
| Sodium Valproate | 200 | 30 | 0.1 ± 0.1 | — | −89%*** |
| Compound 2 | 10 | 30 | 0.7 ± 0.1 | — | −22% |
| Compound 2 | 30 | 15 | 0.7 ± 0.1 | — | −22% |
| Compound 2 | 60 | 30 | 0.7 ± 0.1 | — | −22% |
| Compound 2 | 100 | 30 | 0.3 ± 0.1 | — | −67% |

***p < 0.001; slight sedation observed at 100 mg/kg, which may impact seizure evaluation; no adverse signs were noted in animals following dosing of Compound 2 at 10, 30 or 60 mg/kg i.p.

TABLE 25
6 Hz Partial Seizure Test Results for Compound 4 (n = 20)

| | | Test | | | |
|---|---|---|---|---|---|
| | | time | Mean | Change | Change |
| | | post | Forelimb | from | from |
| | Dose | dose | Clonus +/− | vehicle 1 | vehicle 2 |
| Compound | (mg/kg) | (min) | SEM | (%) | (%) |
| Vehicle 1 (saline) | — | 30 | 1.0 ± 0.14 | — | — |
| Vehicle 2(1:2:17 | — | 30 | 0.65 ± 0.13 | −41%* | — |
| ethanol:Kolliphor | | | | | |
| HS15; saline) | | | | | |
| Sodium Valproate | 200 | 30 | 0.2 ± 0.09 | −82%*** | — |
| Compound 4 | 10 | 30 | 1.00 ± 0.11 | — | +54% |
| Compound 4 | 30 | 15 | 0.30 ± 0.11 | — | −54% |
| Compound 4 | 60 | 30 | 0.30 ± 0.11 | — | −54% |
| Compound 4 | 100 | 30 | 0.11 ± 0.07 | — | −83%** |

*p < 0.05;
**p < 0.01;
***p < 0.001; relatively low forelimb seizure scores in the 1:2:17 vehicle group may impact data interpretation; slight to moderate sedation observed with Compound 4 at 60 and 100 mg/kg.

TABLE 26
6 Hz Partial Seizure Test Results for Compound 11 (n = 20)

| | | Test time | | |
|---|---|---|---|---|
| | Dose | post dose | Mean Forelimb | Change from |
| Compound | (mg/kg) | (min) | Clonus +/− SEM | vehicle (%) |
| Vehicle (HPMC) | — | 30 | 1.00 ± 0.10 | — |
| Sodium Valproate | 200 | 30 | 0.40 ± 0.11 | −60%*** |
| Compound 11 | 25 | 15 | 0.85 ± 0.13 | −15% |
| Compound 11 | 50 | 30 | 0.55 ± 0.11 | −45% |
| Compound 11 | 100 | 30 | 0.80 ± 0.14 | −20% |
| Compound 11 | 200 | 60 | 0.75 ± 0.12 | −25% |

***p < 0.001

### Conclusions

The results shown in Table 24 to Table 26 demonstrate that the test compounds are able to suppress seizures compared to control. This data demonstrates that the test compounds display anticonvulsant activity in a mouse model of partial seizure. Thus, the test compounds may be useful in medical treatment, such as in the treatment of seizure.

### Example 5: Evaluation of the Anticonvulsive Properties of Compounds Using the Mouse Audiogenic Seizure Test

The Audiogenic Seizure Test, which detects anticonvulsant activity, follows that described by Durmüller et al., 1993. Mice of the DBA/2 strain undergo an age-dependent sequence of convulsions (wild-running, clonic convulsion, tonic convulsion and respiratory arrest) within 60 seconds of exposure to a loud sound of 110-120 dB. The type of convulsions are consistent with aspects of generalised seizure in humans.

Antiepileptic drugs such as sodium valproate and carbamazepine protect DBA/2 mice from clonic convulsions, tonic convulsions and respiratory arrest, indicative of an anticonvulsant effect (De Sarro et al., 2017).

### General Method

### Mice:

Male DBA/2 mice (3-4 weeks old) were supplied by Janvier Labs (53940 Le Genest-Saint-Isle, France); weight range 6-13 g at the beginning of the experiment. Animals were acclimatized to the Test Facility for 1 day after delivery and randomly housed in groups of 5 in macrolon cages on wood litter with free access to food and water.

The animal house was maintained under artificial lighting (12 hours) between 7:00 and 19:00 in a controlled ambient temperature of 22±2 °C and relative humidity between 30-70%.

### Study Details:

Mice were individually transferred (at 3-5 minutes intervals) from the preparation room into an adjacent experimental room and body temperature was measured using a rectal thermometer (Digital Laboratory Thermometer: Model BAT-12). Immediately after, they were placed in a Plexiglas jar (Diameter=40 cm; Height=35 cm) mounted with an electric bell (110-120 dB, supplier Leroy Merlin, France). Upon activating the bell, the occurrences and latencies to wild running fits, clonic and tonic seizures were measured. Deaths were also recorded. The bell was activated until a tonic seizure occurred, or for a maximum of 60 seconds.

The experiment included groups of 10 mice per group. Experimenters were only partially blinded to treatment group as pre-treatment times were different.

The test substance was evaluated at various doses, administered i.p. (10 ml/kg) 15 or 30 minutes before the test, and compared with a vehicle control group.

Due to the number of animals tested, the experiment was divided in 2 sub-experiments on 2 separate days (7 days apart) with 5 mice per group in each sub-experiment.

### Sample Collection:

The euthanasia and tissue collection method, which was designed to minimize animal suffering and to ensure good quality of biological samples, was adapted from basic procedures commonly used in studies performed in rodents.

At the end of each sub-experiment, the surviving mice treated with the test substance (4 groups×10 mice) were placed under isoflurane anesthesia (5% for induction and 2% for maintenance, under 100% O₂). Maximum volume of blood was collected by cardiac puncture using a sterile disposable syringe. The blood samples were immediately transferred into pre-labelled tubes containing K2-EDTA. After sealing each tube, the blood samples were manually agitated and stored on ice until centrifugation (within 30 minutes of sampling). The samples were centrifuged at +4 °C, at 1500 g, for 10 minutes. Once separated, the resulting plasma (25 L) was stabilised immediately with 100 mg/mL ascorbic acid (aq) in a 1:1 v/v ratio. Ascorbic acid was prepared fresh on the day of use. The samples were stored at −80 °C until shipment.

Each animal was humanely killed immediately after blood collection in compliance with Council Directive No. 2010/63/UE of Sep. 22, 2010 on the protection of animals used for scientific purposes and French decree No. 2013-118 of Feb. 1, 2013 on the protection of animals Animal Health regulations. The brain hemispheres were dissected, weighed, placed in separate pre-labelled vials and snap frozen on liquid nitrogen. The vials were stored upright at approximately −80 °C and protected from light until transport to the bioanalysis site for analysis.

### Statistical Analysis:

Data were tested for normality using d'Agostino-Pearson Test and found to not follow a normal distribution. Statistical outliers were not identified or removed because no behavioural outliers were identified. Quantitative data (latencies) with the test substance were analyzed by comparing treated groups with vehicle control using Kruskal-Wallis test followed by Dunn's multiple comparisons test. Quantitative data with the reference substance were analyzed using Mann-Whitney U test. Quantal data (frequencies) were analyzed by comparing treated groups with vehicle control using Fisher's Exact Probability test.

### Results

Table 27 to Table 29 show the test results for Compound 2 and Table 30 to Table 32 Table 1 show the test results for Compound 4.

TABLE 27
Audiogenic Seizure Test (wild running) for Compound 2 (n = 10)

| | | | Wild running - | | Wild running - | |
|---|---|---|---|---|---|---|
| | | | presence | | latency | |
| | | Test time | | Change | | Change |
| | | Post- | | from | | from |
| | Dose | dose | | vehicle | Median:IQR- | vehicle |
| Compound | (mg/kg) | (min) | Total | (%) | Q1:IQR-Q3 | (%) |
| Vehicle (1:2:17 | — | 30 | 9 | — | 5.1:4.0:5.7 | — |
| ethanol:Kolliphor | | | | | | |
| HS15; saline) | | | | | | |
| Sodium Valproate | 180 | 30 | 1 | −89%** | 60.0:60.0:60.0 | +1076%*** |
| Compound 2 | 3 | 30 | 10 | +11% | 5.9:4.3:6.3 | +16% |
| Compound 2 | 10 | 30 | 8 | −11% | 6.5:5.4:22.3 | +27% |
| Compound 2 | 30 | 15 | 2 | −78%** | 60.0:48.9:60.0 | +1076%*** |

**p < 0.01;
***p < 0.001

Valproate (180 mg/kg), administered i.p. 30 minutes before the test, significantly decreased the number of mice displaying wild running and significantly increased the latencies to wild running. Compound 2 at 30 mg/kg significantly decreased the number of mice displaying wild running (−78%, p<0.01). There were no significant differences on the number of mice displaying wild running at 3 or 10 mg/kg. Dunn's multiple comparisons test showed that Compound 2 at 30 mg/kg significantly increased latency to wild running as compared to vehicle (p<0.001). There were no significant differences at 3 and 10 mg/kg.

TABLE 28
Audiogenic Seizure Test (clonic convulsions) for Compound 2 (n = 10)

| | | | Clonic convulsion - | | Clonic convulsion - | |
|---|---|---|---|---|---|---|
| | | | presence | | latency | |
| | | Test time | | Change | | Change |
| | | Post- | | from | | from |
| | Dose | dose | | vehicle | Median:IQR- | vehicle |
| Compound | (mg/kg) | (min) | Total | (%) | Q1:IQR-Q3 | (%) |
| Vehicle (1:2:17 | — | 30 | 8 | — | 8.9:8.0:22.7 | — |
| ethanol:Kolliphor | | | | | | |
| HS15; saline) | | | | | | |
| Sodium Valproate | 180 | 30 | 1 | −88%** | 60.0:60.0:60.0 | +574%** |
| Compound 2 | 3 | 30 | 10 | +25% | 9.8:7.6:12.7 | +10% |
| Compound 2 | 10 | 30 | 7 | −13% | 12.8:9.7:60.0 | +44% |
| Compound 2 | 30 | 15 | 2 | −75%* | 60.0:49.8:60.0 | +574%** |

*p < 0.05;
**p < 0.01;
***p < 0.001

Valproate (180 mg/kg), administered i.p. 30 minutes before the test, significantly decreased the number of mice displaying clonic convulsions and significantly increased the latencies to clonic convulsions. Compound 2 at 30 mg/kg significantly decreased the number of mice displaying clonic convulsions (−75%, p<0.05). There were no significant differences on the number of mice displaying clonic convulsions at 3 or 10 mg/kg. Dunn's multiple comparisons test showed that Compound 2 at 30 mg/kg significantly increased latency to clonic convulsions as compared to vehicle (p<0.01). There were no significant differences at 3 and 10 mg/kg.

TABLE 29
Audiogenic Seizure Test (tonic convulsions) for Compound 2 (n = 10)

| | | | Tonic convulsion - | | Tonic convulsion - | |
|---|---|---|---|---|---|---|
| | | | presence | | latency | |
| | | Test time | | Change | | Change |
| | | Post- | | from | | from |
| | Dose | dose | | vehicle | Median:IQR- | vehicle |
| Compound | (mg/kg) | (min) | Total | (%) | Q1:IQR-Q3 | (%) |
| Vehicle (1:2:17 | — | 30 | 7 | — | 12.0:11.1:60.0 | — |
| ethanol:Kolliphor | | | | | | |
| HS15; saline) | | | | | | |
| Sodium Valproate | 180 | 30 | 0 | −100%** | 60.0:60.0:60.0 | +400%** |
| Compound 2 | 3 | 30 | 8 | +14% | 14.7:11.5:29.7 | +23% |
| Compound 2 | 10 | 30 | 3 | −57% | 60.0:20.1:60.0 | +400%* |
| Compound 2 | 30 | 15 | 0 | −100%** | 60.0:60.0:60.0 | +400%** |

*p < 0.05;
**p < 0.01

Valproate (180 mg/kg), administered i.p. 30 minutes before the test, significantly decreased the number of mice displaying tonic convulsions and significantly increased the latencies to tonic convulsions. Compound 2 at 30 mg/kg significantly decreased the number of mice displaying tonic convulsions (−100%, p<0.01). There were no significant differences on the number of mice displaying tonic convulsions at 3 or 10 mg/kg. Dunn's multiple comparisons test showed that Compound 2 at 10 and 30 mg/kg significantly increased latency to tonic convulsions as compared to vehicle (p<0.05 and p<0.01 respectively). There were no significant differences at 3 mg/kg.

TABLE 30
Audiogenic Seizure Test (wild running) for Compound 4 (n = 10)

| | | | Wild running - | | Wild running - | |
|---|---|---|---|---|---|---|
| | | | presence | | latency | |
| | | Test time | | Change | | Change |
| | | Post- | | from | | from |
| | Dose | dose | | vehicle | Median:IQR- | vehicle |
| Compound | (mg/kg) | (min) | % | (%) | Q1:IQR-Q3 | (%) |
| Vehicle (1:2:17 | — | 30 | 100 | — | 4.8:4.5:5.4 | — |
| ethanol:Kolliphor | | | | | | |
| HS15; saline) | | | | | | |
| Sodium Valproate | 180 | 30 | 30 | −70%** | 60.0:36.5:60.0 | +1150%*** |
| Compound 4 | 3 | 30 | 100 | 0% | 4.7:3.7:5.3 | −2% |
| Compound 4 | 10 | 30 | 100 | 0% | 5.0:3.7:6.2 | +4% |
| Compound 4 | 30 | 15 | 70 | −30% | 34.0:8.8:60.0 | +608%** |
| Compound 4 (n = 9) | 60 | 30 | 22 | −80%*** | 60.0:48.5:60.0 | +1150%*** |

**p < 0.01;
***p < 0.001

Valproate (180 mg/kg), administered i.p. 30 minutes before the test, significantly decreased the number of mice displaying wild running and significantly increased the latencies to wild running. Compound 4 at 60 mg/kg significantly decreased the number of mice displaying wild running (−80%, p<0.001). There were no significant differences on the number of mice displaying wild running at 3, 10 or 30 mg/kg. Dunn's multiple comparisons test showed that Compound 4 at 30 and 60 mg/kg significantly increased latency to wild running as compared to vehicle (p<0.01 and p<0.001 respectively). There were no significant differences at 3 and 10 mg/kg.

TABLE 31
Audiogenic Seizure Test (clonic convulsions) for Compound 4 (n = 10)

| | | | Clonic convulsion - | | Clonic convulsion - | |
|---|---|---|---|---|---|---|
| | | | presence | | latency | |
| | | Test time | | Change | | Change |
| | | Post- | | from | | from |
| | Dose | dose | | vehicle | Median:IQR- | vehicle |
| Compound | (mg/kg) | (min) | % | (%) | Q1:IQR-Q3 | (%) |
| Vehicle (1:2:17 | — | 30 | 100 | — | 8.4:6.4:8.9 | — |
| ethanol:Kolliphor | | | | | | |
| HS15; saline) | | | | | | |
| Sodium Valproate | 180 | 30 | 30 | −70%** | 60.0:38.7:60.0 | +614%*** |
| Compound 4 | 3 | 30 | 100 | 0% | 8.0:7.5:9.4 | −5% |
| Compound 4 | 10 | 30 | 90 | −10% | 8.8:7.8:11.8 | +5% |
| Compound 4 | 30 | 15 | 70 | −30% | 39.3:12.1:60.0 | +368%** |
| Compound 4 (n = 9) | 60 | 30 | 11 | −90%*** | 60.0:60.0:60.0 | +614%*** |

**p < 0.01;
***p < 0.001

Valproate (180 mg/kg), administered i.p. 30 minutes before the test, significantly decreased the number of mice displaying clonic convulsions and significantly increased the latencies to clonic convulsions. Compound 4 at 60 mg/kg significantly decreased the number of mice displaying clonic convulsions (−90%, p<0.001). There were no significant differences on the number of mice displaying clonic convulsions at 3, 10 or 30 mg/kg. Dunn's multiple comparisons test showed that Compound 4 at 30 and 60 mg/kg significantly increased latency to clonic convulsions as compared to vehicle (p<0.01 and p<0.001 respectively). There were no significant differences at 3 and 10 mg/kg.

TABLE 32
Audiogenic Seizure Test (tonic convulsions) for Compound 4 (n = 10)

| | | | Tonic convulsion - | | Tonic convulsion - | |
|---|---|---|---|---|---|---|
| | | | presence | | latency | |
| | | Test time | | Change | | Change |
| | | Post- | | from | | from |
| | Dose | dose | | vehicle | Median:IQR- | vehicle |
| Compound | (mg/kg) | (min) | % | (%) | Q1:IQR-Q3 | (%) |
| Vehicle (1:2:17 | — | 30 | 100 | — | 11.4:8.6:12.4 | — |
| ethanol:Kolliphor | | | | | | |
| HS15; saline) | | | | | | |
| Sodium Valproate | 180 | 30 | 0 | −100%*** | 60.0:60.0:60.0 | +426%*** |
| Compound 4 | 3 | 30 | 90 | −10% | 12.1:10.7:14.8 | +6% |
| Compound 4 | 10 | 30 | 70 | −30% | 14.1:10.8:60.0 | +24% |
| Compound 4 | 30 | 15 | 10 | −90%*** | 60.0:60.0:60.0 | +426%*** |
| Compound 4 (n = 9) | 60 | 30 | 0 | −100%*** | 60.0:60.0:60.0 | +426%*** |

***p < 0.001

Valproate (180 mg/kg), administered i.p. 30 minutes before the test, significantly decreased the number of mice displaying tonic convulsions and significantly increased the latencies to tonic convulsions. Compound 4 at 30 and 60 mg/kg significantly decreased the number of mice displaying tonic convulsions (−90% and −100% respectively, p<0.01). There were no significant differences on the number of mice displaying tonic convulsions at 3 or 10 mg/kg. Dunn's multiple comparisons test showed that Compound 4 at 30 and 60 mg/kg significantly increased latency to tonic convulsions as compared to vehicle (p<0.001). There were no significant differences at 3 or 10 mg/kg.

### Conclusions

The results shown in Table 27 to Table 32 demonstrate that the test compounds display anticonvulsant activity in a model of generalised seizure using mice prone to audiogenic seizures The test compounds protected the mice from multiple seizure endpoints (wild running, clonic convulsions, and tonic convulsions). Thus, the test compounds may be useful in medical treatment, such as in the treatment of epilepsy.

### Biological Tests—Conclusions

The above results described in Examples 2 to 5 demonstrate that the test compounds display anti-convulsive activity in several different and complementary mice models of seizure (mini-MEST; MEST; 6 Hz and audiogenic seizure models). Thus, the compounds of formula (I) may be useful in medical treatment, such as in the treatment of seizure.

### REFERENCES

A number of publications are cited above in order to more fully describe and disclose the invention and the state of the art to which the invention pertains. Full citations for these references are provided below. The contents of each of these references is incorporated herein.
1. Devinsky et al., “Epilepsy”, Nature Reviews Disease Primers, 2018, 3, article number 18024.
2. Fiest et al., “Prevalence and incidence of epilepsy: a systematic review and meta-analysis of international studies”, Neurology, 2017, 88, 296-303.
3. Scheffer et al., “ILAE classification of the epilepsies: Position paper of the ILAE Commission for Classification and Terminology” Epilepsia, 2017, 58(4), 512-521.
4. Fisher et al., “Operational classification of seizure types by the ILAE: Position paper of the ILAE Commission for Classification and Terminology” Epilepsia, 2017, 58(4), 522-530.
5. Wirrell et al., “Methodology for classification and definition of epilepsy syndromes with list of syndromes: Report of the ILAE Task Force on Nosology and Definitions” Epilepsia, 2022, 63, 1333-1348.
6. Zuberi et al., “ILAE classification and definition of epilepsy syndromes with onset in neonates and infants: Position statement by the ILAE Task Force on Nosology and Definitions” Epilepsia, 2022, 63, 1349-1397.
7. Specchio et al., “ILAE classification and definition of epilepsy syndromes with onset in childhood: Position statement by the ILAE Task Force on Nosology and Definitions” Epilepsia, 2022, 63, 1398-1442.
8. Riney et al., “ILAE classification and definition of epilepsy syndromes with onset at a variable age: Position statement by the ILAE Task Force on Nosology and Definitions” Epilepsia, 2022, 63, 1443-1474.
9. Hirsch et al., “ILAE definition of Idiopathic Generalized Epilepsy Syndromes: Position statement by the ILAE Task Force on Nosology and Definitions” Epilepsia, 2022, 63, 1475-1499.
10. Löscher et al., “The role of technical, biological and pharmacological factors in the laboratory evaluation of anticonvulsant drugs. II. Maximal electroshock seizure models.”, Epilepsy Res., 1991, Vol. 8, 79-84.
11. Kimball et al., “Chemical protection against ionizing radiation: I. Sampling methods for screening compounds in radiation protection studies in mice.”, Radiat. Res., 1957, Vol. 7, 1-12.
12. Brown et al., “Comparative assay of antiepileptic drugs by ‘psychomotor’ seizure test and minimal electroshock threshold test”, J. Pharmacol. Exp. Ther. 1953, 107, 273-283.
13. Klein et al., “Evaluation of Cannabidiol in Animal Seizure Models by the Epilepsy Therapy Screening Program (ETSP)”, Neurochem Res, 2017, 42(7), 1939-1948.
14. Dürmüller et al., “Proconvulsant and anticonvulsant effects of Evans blue dye in rodents”, NeuroReport, 1993, 4, 683-686.
15. De Sarro et al., “Genetically epilepsy-prone rats (GEPRs) and DBA/2 mice: Two animal models of audiogenic reflex epilepsy for the evaluation of new generation AEDs”, Epilepsy & Behavior, 2017, 71, 165-173.

1. A compound of formula (I), or a salt thereof: 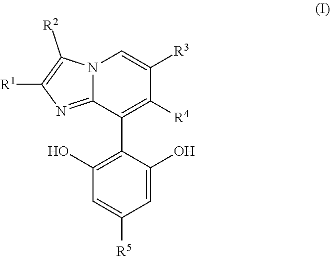
wherein,
R¹ is hydrogen, C₁₋₃alkyl, C₁₋₃alkoxy, C₁₋₃haloalkyl, or halo;
R² is hydrogen, C₁₋₃alkyl, C₁₋₃alkoxy, or halo;
R³ is hydrogen, C₁₋₃alkyl, C₁₋₃alkoxy, C₁₋₃haloalkyl, C₃₋₆cycloalkyl, or halo;
R⁴ is hydrogen, C₁₋₃alkyl, C₁₋₃alkoxy, C₁₋₃haloalkyl, C₃₋₆cycloalkyl, or halo; and
R⁵ is hydrogen, C₁₋₁₀alkyl, C₁₋₁₀haloalkyl, C₃₋₆cycloalkyl, or halo.

2. The compound according to claim 1, wherein R¹ is hydrogen, C₁₋₃alkyl, C₁₋₃haloalkyl, or halo; such as hydrogen, methyl, CF₃, or chloro.

3. The compound according to claim 1, wherein R¹ is hydrogen.

4. The compound according to any of claims 1 to 3, wherein R² is hydrogen, C₁₋₃alkyl, or halo; such as hydrogen, methyl, or chloro.

5. The compound according to any of claims 1 to 3, wherein R² is hydrogen.

6. The compound according to any of claims 1 to 5, wherein R³ is hydrogen, C₁₋₃alkyl, C₁₋₃ haloalkyl, C₃₋₆cycloalkyl, or halo; such as hydrogen, methyl, CF₃, cyclopropyl, fluoro, or chloro.

7. The compound according to any of claims 1 to 5, wherein R³ is hydrogen, fluoro, or chloro.

8. The compound according to any of claims 1 to 7, wherein R⁴ is hydrogen, C₁₋₃alkyl, C₁₋₃haloalkyl, C₃₋₆cycloalkyl, or halo; such as hydrogen, methyl, CF₃, cyclopropyl, fluoro, or chloro.

9. The compound according to any of claims 1 to 7, wherein R⁴ is methyl, CF₃, cyclopropyl, fluoro, or chloro.

10. The compound according to any of claims 1 to 7, wherein R⁴ is methyl.

11. The compound according to any of claims 1 to 10, wherein R⁵ is:
a. C₁₋₁₀alkyl, C₁₋₁₀haloalkyl, C₃₋₆cycloalkyl, or halo;
b. C₃₋₆alkyl, C₃₋₆haloalkyl, C₃₋₆cycloalkyl, or halo; or
C. C₃₋₅alkyl, C₃₋₅haloalkyl, cyclopentyl, or chloro.

12. The compound according to any of claims 1 to 10, wherein R⁵ is propyl, pentyl, 1,1-dimethylpropyl, 3,3,3-trifluoropropyl, cyclopentyl, or chloro.

13. The compound according to any of claims 1 to 10, wherein R⁵ is propyl.

14. The compound according to claim 1, wherein the compound is a compound of any of formulae (IA) to (IF): 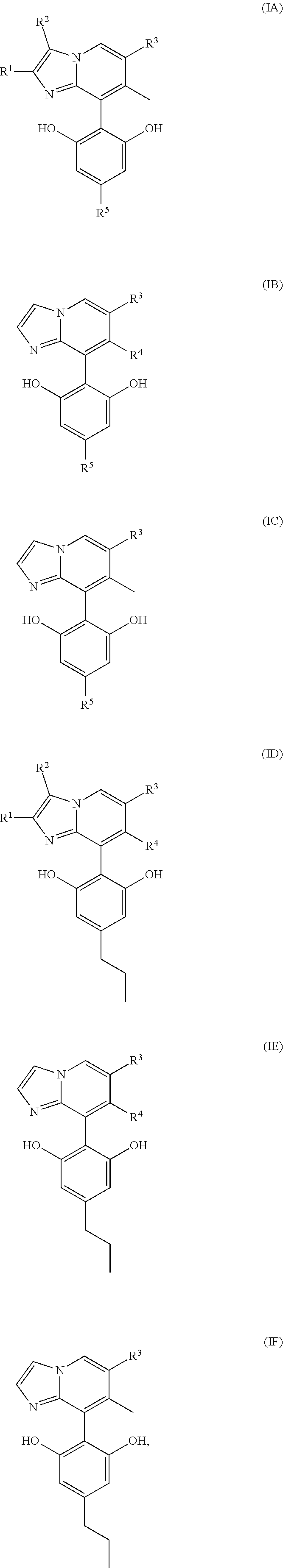
wherein R¹, R², R³, R⁴, and R⁵ are as defined in any of claims 1 to 13.

15. The compound according to claim 14, wherein the compound is a compound of formula (IC) 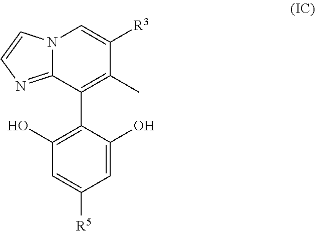
wherein,
R³ is hydrogen, fluoro, or chloro; and
R⁵ is C₃₋₆alkyl.

16. The compound according to claim 15, wherein R⁵ is propyl.

17. The compound according to claim 1, wherein the compound is selected from the following compounds and salts thereof:
2-(imidazo[1,2-a]pyridin-8-yl)-5-propylbenzene-1,3-diol;
2-(7-methylimidazo[1,2-a]pyridin-8-yl)-5-propylbenzene-1,3-diol;
2-(2,7-dimethylimidazo[1,2-a]pyridin-8-yl)-5-propylbenzene-1,3-diol;
2-(6-chloro-7-methylimidazo[1,2-a]pyridin-8-yl)-5-propylbenzene-1,3-diol;
2-(7-trifluoromethylimidazo[1,2-a]pyridin-8-yl)-5-propylbenzene-1,3-diol;
2-(7-chloroimidazo[1,2-a]pyridin-8-yl)-5-propylbenzene-1,3-diol;
2-(6,7-dimethylimidazo[1,2-a]pyridin-8-yl)-5-propylbenzene-1,3-diol;
2-(7-fluoroimidazo[1,2-a]pyridin-8-yl)-5-propylbenzene-1,3-diol;
2-(3,7-dimethylimidazo[1,2-a]pyridin-8-yl)-5-propylbenzene-1,3-diol;
2-(7-cyclopropylimidazo[1,2-a]pyridin-8-yl)-5-propylbenzene-1,3-diol;
2-(3-chloro-7-methylimidazo[1,2-a]pyridin-8-yl)-5-propylbenzene-1,3-diol;
2-(6-fluoro-7-methylimidazo[1,2-a]pyridin-8-yl)-5-propylbenzene-1,3-diol;
2-(2-chloro-7-methylimidazo[1,2-a]pyridin-8-yl)-5-propylbenzene-1,3-diol;
2-(7-methylimidazo[1,2-a]pyridin-8-yl)-5-pentylbenzene-1,3-diol;
5-Cyclopentyl-2-(7-methylimidazo[1,2-a]pyridin-8-yl)benzene-1,3-diol;
2-(2-trifluoromethyl-7-methylimidazo[1,2-a]pyridin-8-yl)-5-propylbenzene-1,3-diol;
2-(6-cyclopropyl-7-methylimidazo[1,2-a]pyridin-8-yl)-5-propylbenzene-1,3-diol;
2-(6-trifluoromethyl-7-methylimidazo[1,2-a]pyridin-8-yl)-5-propylbenzene-1,3-diol;
2-(6-chloro-7-methylimidazo[1,2-a]pyridin-8-yl)-5-pentylbenzene-1,3-diol;
2-(7-Methylimidazo[1,2-a]pyridin-8-yl)benzene-1,3-diol;
2-(7-methylimidazo[1,2-a]pyridin-8-yl)-5-(tert-pentyl)benzene-1,3-diol;
2-(6-chloro-7-methylimidazo[1,2-a]pyridin-8-yl)-5-(tert-pentyl)benzene-1,3-diol;
5-Chloro-2-(6-chloro-7-methylimidazo[1,2-a]pyridin-8-yl)benzene-1,3-diol;
2-(6-Fluoro-7-methylimidazo[1,2-a]pyridin-8-yl)-5-(3,3,3-trifluoropropyl)benzene-1,3-diol; and
2-(6-Chloroimidazo[1,2-a]pyridin-8-yl)-5-propylbenzene-1,3-diol.

18. A pharmaceutical composition comprising a compound according to of any of claims 1 to 17, or a pharmaceutically acceptable salt thereof, together with one or more ingredients selected from carriers, diluents, excipients, adjuvants, fillers, buffers, binders, disintegrants, preservatives, antioxidants, lubricants, stabilisers, solubilisers, surfactants, masking agents, colouring agents, flavouring agents, and sweetening agents.

19. A compound according to any of claims 1 to 17, a salt thereof, or a pharmaceutical composition according to claim 18, for use in a method of treatment.

20. The compound or the pharmaceutical composition for the use according to claim 19, wherein the treatment is the treatment of a condition associated with seizure.

21. The compound or the pharmaceutical composition for the use according to claim 20, wherein the condition associated with seizure is selected from focal onset seizure, generalised onset seizure, and unknown onset seizure.

22. The compound or the pharmaceutical composition for the use according to claim 20, wherein the condition associated with seizure is a seizure type selected from absence seizure, atypical absence seizure, atonic seizure, clonic seizure, tonic seizure, tonic-clonic seizure, febrile seizure, focal to bilateral tonic clonic seizure, gelastic and dacrystic seizure, myoclonic seizure, myoclonic-tonic-clonic seizure, myoclonic-atonic seizure, and epileptic (or infantile) spasms.

23. The compound or the pharmaceutical composition for the use according to claim 20, wherein the condition associated with seizure is an epilepsy selected from focal epilepsy, generalised epilepsy, and combined generalised & focal epilepsy.

24. The compound or the pharmaceutical composition for the use according to claim 20, wherein the condition associated with seizure is an epilepsy syndrome selected from syndromes with onset in neonates and infancy, syndromes with onset in childhood, syndromes with onset at a variable age, and idiopathic generalised epilepsy syndromes (IGEs).

25. The compound or the pharmaceutical composition for use according to claim 20, wherein the condition associated with seizure is an epilepsy having an aetiology selected from structural, genetic, infection, metabolic, and autoimmune.

26. A method of treatment comprising administering to a subject in need of treatment a therapeutically effective amount of a compound according to any of claims 1 to 17, a salt thereof, or a pharmaceutical composition according to claim 18.

27. The method according to claim 26, wherein the treatment is treatment of a condition associated with seizure.

28. The method according to claim 27, wherein the condition associated with seizure is selected from focal onset seizure, generalised onset seizure, and unknown onset seizure.

29. The method according to claim 27, wherein the condition associated with seizure is a seizure type selected from absence seizure, atypical absence seizure, atonic seizure, clonic seizure, tonic seizure, tonic-clonic seizure, febrile seizure, focal to bilateral tonic clonic seizure, gelastic and dacrystic seizure, myoclonic seizure, myoclonic-tonic-clonic seizure, myoclonic-atonic seizure, and epileptic (or infantile) spasms.

30. The method according to claim 27, wherein the condition associated with seizure is selected from focal epilepsy, generalised epilepsy, and combined generalised & focal epilepsy.

31. The method according to claim 27, wherein the condition associated with seizure is an epilepsy syndrome selected from syndromes with onset in neonates and infancy, syndromes with onset in childhood, syndromes with onset at a variable age, and idiopathic generalised epilepsy syndromes (IGEs).

32. The method according to claim 27, wherein the condition associated with seizure is an epilepsy having an aetiology selected from structural, genetic, infection, metabolic, and autoimmune.

33. Use of a compound according to any of claims 1 to 17, a salt thereof, or a pharmaceutical composition according to claim 18, for the manufacture of a medicament.

34. Use of a compound according to any of claims 1 to 17, a salt thereof, or a pharmaceutical composition according to claim 18, in a method of treatment.

35. A method of preparing a compound of formula (I) according to claim 1, the method comprising:
(1a) reacting a compound of formula (II) with a compound of formula (III) to give a compound of formula (IV): 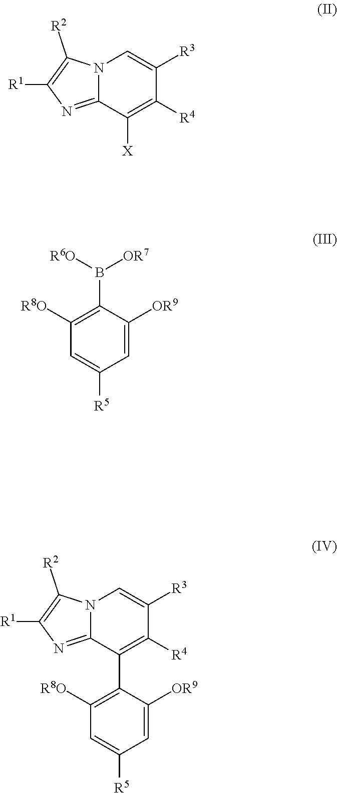
where:
R¹, R², R³, R⁴, and R⁵ are as defined in claim 1;
X is chloro, bromo, iodo, or triflate;
R⁶ and R⁷ are hydrogen, alkyl or phenyl; or R⁶ and R⁷ are joined to form a cyclic boronic ester (such as pinacol, neopentyl or catechol boronic esters); and
R⁸ and R⁹ are suitable alcohol protecting groups such as methyl or benzyl; and
(1b) converting a compound of formula (IV) to a compound of formula (I).

36. The method according to claim 35, wherein R⁶ and R⁷ are both hydrogen; or R⁶ and R⁷ together form

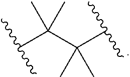 [SMILES: *C(C)(C)C(*)(C)C]

37. The method according to claim 35 or claim 36, wherein R⁸ and R⁹ are both methyl; or R⁸ and R⁹ are both benzyl.

38. The method according to any one of claims 35 to 37, wherein step (1a) comprises reacting a compound of formula (II) with a compound of formula (III) and a palladium catalyst, and optionally a base.

39. The method according to any one of claims 35 to 38, wherein step (1a) is carried out at a temperature of from 60 °C to 140 °C.

40. A compound obtained or obtainable by the method according to any one of claims 35 to 39.

41. A compound of formula (II): 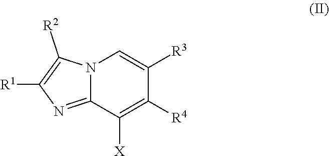
wherein R¹ is hydrogen, C₁₋₃alkyl, C₁₋₃alkoxy, C₁₋₃haloalkyl, or halo;
R² is hydrogen, C₁₋₃alkyl, C₁₋₃alkoxy, or halo;
R³ is hydrogen, C₁₋₃alkyl, C₁₋₃alkoxy, C₁₋₃haloalkyl, C₃₋₆cycloalkyl, or halo;
R⁴ is hydrogen, C₁₋₃alkyl, C₁₋₃alkoxy, C₁₋₃haloalkyl, C₃₋₆cycloalkyl, or halo; and
X is chloro, bromo, iodo, or triflate.

42. The compound of formula (II) according to claim 41 selected from:
8-Bromo-7-methylimidazo[1,2-a]pyridine;
8-Bromo-2,7-dimethylimidazo[1,2-a]pyridine;
8-Bromo-6-chloro-7-methylimidazo[1,2-a]pyridine;
8-Chloro-7-(trifluoromethyl) imidazo[1,2-a]pyridine;
8-Bromo-6,7-dimethylimidazo[1,2-a]pyridine;
8-Bromo-7-fluoroimidazo[1,2-a]pyridine;
8-Bromo-3,7-dimethylimidazo[1,2-a]pyridine;
8-Bromo-7-cyclopropylimidazo[1,2-a]pyridine;
8-Bromo-3-chloro-7-methylimidazo[1,2-a]pyridine;
8-Bromo-6-fluoro-7-methylimidazo[1,2-a]pyridine;
8-Bromo-2-chloro-7-methylimidazo[1,2-a]pyridine;
8-Bromo-2-(trifluoromethyl)-7-methylimidazo[1,2-a]pyridine; and
8-Bromo-6-(trifluoromethyl)-7-methylimidazo[1,2-a]pyridine.

43. A compound of formula (IV): 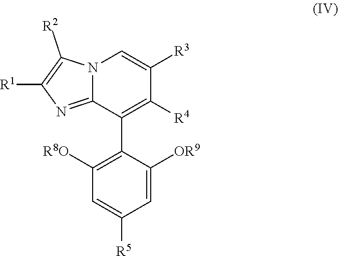
wherein R¹ is hydrogen, C₁₋₃alkyl, C₁₋₃alkoxy, C₁₋₃haloalkyl, or halo;
R² is hydrogen, C₁₋₃alkyl, C₁₋₃alkoxy, or halo;
R³ is hydrogen, C₁₋₃alkyl, C₁₋₃alkoxy, C₁₋₃haloalkyl, C₃₋₆cycloalkyl, or halo;
R⁴ is hydrogen, C₁₋₃alkyl, C₁₋₃alkoxy, C₁₋₃haloalkyl, C₃₋₆cycloalkyl, or halo;
R⁵ is hydrogen, C₁₋₁₀alkyl, C₁₋₁₀haloalkyl, C₃₋₆cycloalkyl, or halo; and
R⁸ and R⁹ are suitable alcohol protecting groups (such as methyl or benzyl).

44. The compound of formula (IV) according to claim 43 selected from:
8-(2,6-Dimethoxy-4-propylphenyl) imidazo[1,2-a]pyridine;
8-(2,6-Dimethoxy-4-propylphenyl)-7-methylimidazo[1,2-a]pyridine;
8-(2,6-Dimethoxy-4-propylphenyl)-2,7-dimethylimidazo[1,2-a]pyridine;
6-Chloro-8-(2,6-dimethoxy-4-propylphenyl)-7-methylimidazo[1,2-a]pyridine;
8-(2,6-Dimethoxy-4-propylphenyl)-7-trifluoromethylimidazo[1,2-a]pyridine;
7-Chloro-8-(2,6-dimethoxy-4-propylphenyl) imidazo[1,2-a]pyridine;
8-(2,6-Dimethoxy-4-propylphenyl)-6,7-dimethylimidazo[1,2-a]pyridine;
7-Fluoro-8-(2,6-dimethoxy-4-propylphenyl) imidazo[1,2-a]pyridine;
8-(2,6-Dimethoxy-4-propylphenyl)-3,7-dimethylimidazo[1,2-a]pyridine;
3-Chloro-8-(2,6-dimethoxy-4-propylphenyl)-7-methylimidazo[1,2-a]pyridine;
6-Fluoro-8-(2,6-dimethoxy-4-propylphenyl)-7-methylimidazo[1,2-a]pyridine;
2-Chloro-8-(2,6-dimethoxy-4-propylphenyl)-7-methylimidazo[1,2-a]pyridine;
8-(2,6-Dimethoxy-4-pentylphenyl) 7-methylimidazo[1,2-a]pyridine;
8-(2,6-Dimethoxyphenyl)-7-methylimidazo[1,2-a]pyridine;
8-(2,6-Dimethoxy-4-propylphenyl)-2-trifluoromethyl-7-methylimidazo[1,2-a]pyridine;
8-(2,6-Dimethoxy-4-propylphenyl)-6-trifluoromethyl-7-methylimidazo[1,2-a]pyridine;
6-Chloro-8-(2,6-dimethoxy-4-pentylphenyl)-7-methylimidazo[1,2-a]pyridine;
8-(2,6-Dimethoxy-4-propylphenyl)-7-cyclopropylimidazo[1,2-a]pyridine;
6-Chloro-8-(4-chloro-2,6-dimethoxyphenyl)-7-methylimidazo[1,2-a]pyridine;
6-Chloro-8-(2,6-dimethoxy-4-tert-pentylphenyl)-7-methylimidazo[1,2-a]pyridine;
8-(2,6-dimethoxy-4-tert-pentylphenyl)-7-methylimidazo[1,2-a]pyridine;
8-(4-Cyclopentyl-2,6-dimethoxyphenyl)-7-methylimidazo[1,2-a]pyridine;
6-Cyclopropyl-8-(2,6-dimethoxy-4-propylphenyl)-7-methylimidazo[1,2-a]pyridine;
8-(2,6-Dimethoxy-4-(3,3,3-trifluoropropyl)phenyl)-6-fluoro-7-methylimidazo[1,2-a]pyridine; and
6-Chloro-8-(2,6-dimethoxy-4-propylphenyl)imidazo[1,2-a]pyridine.
